(19) United States
(12) Patent Application Publication
(10) Pub. No.: US 2023/0225332 A1
(43) Pub. Date: Jul. 20, 2023

(54) COMPOSITIONS COMPRISING AROMATIC DIPEPTIDES-BASED STRUCTURES ENCAPSULATING AN ESTERASE AND USES THEREOF

(71) Applicants: Migal Galilee Research Institute Ltd., Kiryat Shmona (IL); Ramot at Tel-Aviv University Ltd., Tel Aviv (IL)

(72) Inventors: Lihi ADLER-ABRAMOVICH, Tel Aviv (IL); Livnat AFRIAT-JURNOU, Kfar-Szold (IL)

(21) Appl. No.: 18/002,251

(22) PCT Filed: Jun. 16, 2021
(86) PCT No.: PCT/IL2021/050725

§ 371 Date: Dec. 16, 2022

Related U.S. Application Data
(60) Provisional application No. 63/040,299, filed on Jun. 17, 2020.

(51) Int. Cl. A01N 63/50; C12N 9/16; C12N 11/04; A01N 25/34; A01P 1/00

(52) U.S. Cl. CPC A01N 63/50; C12N 9/16; C12Y 301/01025; C12Y 301/08001; C12N 11/04; A01N 25/34; A01P 1/00

(57) ABSTRACT

A composition including nano- or microspheres, and/or tubular nanostructures, each made of a plurality of aromatic dipeptides including end-capping modified aromatic dipeptides, non-modified aromatic dipeptides, or a combination thereof; and encapsulating an esterase or a functional fragment thereof; as well as methods of use. The plurality of aromatic dipeptides may include the end-capping modified aromatic dipeptides only.

### TECHNICAL FIELD

The present invention provides compositions comprising aromatic dipeptides-based structures such as nano- or microspheres, and/or tubular nanostructures, encapsulating an esterase, and methods utilizing said compositions.

### BACKGROUND ART

Most natural enzymes are only marginally stable when taken out of their natural environment, and therefore impractical in agricultural and clinical use (Goldenzweig et al., 2016).

One of the main challenges in moving towards a broader use of enzymes, is increasing their production, stability, heat resistance, formulation, and shelf life, while maintaining high activity, specificity, and/or efficacy.

One option is to use thermostable enzymes with the desired specificity. Another option is to improve the thermal stability and purification yield of an active enzyme with the desired specificity using rational design and/or directed enzyme evolution, by randomly mutating genes and screening populations in an iterative process, in order to select the best performing variants. For instance, WO 2020/255131 discloses mutated phosphotriesterase-like lactonases (PLLs) or functional fragments thereof, as well as methods for treating or preventing a bacterial infection in a host, such as a plant or a part, organ or a plant propagation material thereof, by application of said mutated PLLs or the wild-type enzyme corresponding thereto. PLLs degrade N-acyl homoserine lactones (AHLs) (Afriat et al., 2006) which are the most common autoinducers among gram-negative bacteria that involved in quorum sensing (Poonguzhall el al., 2007). Quorum sensing was also shown to control genes that mediate bacterial pathogenicity, colonization of mammalian host surfaces, and adaptation to different environments (Bassler 1999; Persat et al., 2015; Waters and Bassler, 2005; Miller and Bassler, 2001).

One of the ways for extending the durability of the enzymes is by encapsulation. U.S. Pat. No. 9,790,254 discloses nanostructures made up from two or more types of aromatic dipeptides, which differ from one another by the presence (or absence) of an end-capping moiety. The disclosed nanostructures exhibit a closed tubular structure, short average length and narrow length distribution. Specifically, U.S. Pat. No. 9,790,254 suggests using the disclosed nanostructures for, e.g., enzyme encapsulation.

Products of peptide self-assembly have been widely studied for biomedical applications, including as platforms for drug delivery systems capable of entrapping various materials (Marchesan et al., 2015). These encapsulation capabilities, together with the biocompatibility and biodegradability, have suggested that peptide-based nanostructures could be suitable for the encapsulation and immobilization of enzymes (Park et al., 2010), which are otherwise unstable and sensitive to environmental changes (Karthick et al., 2012). Encapsulation is a favored method because it possesses the lowest chances of modifying the structure of the enzyme and thus preserves its activity (Homaei et al., 2013). Diphenylalanine peptide nanostructures were used to encapsulate curcumin, and then mediate its sustained release (Khadeja et al., 2019). Moreover, horseradish peroxidase (HRP) has been encapsulated within various peptide-based nanostructures to produce biocatalysts and biosensors (Park et al., 2012a). HRP encapsulated within diphenylalanine nanotubes maintained high levels of activity even after prolonged storage, or exposure to relatively high temperatures, or a denaturant (Park et al., 2012b).

### SUMMARY OF INVENTION

In one aspect, the present invention provides a composition comprising a plurality of nano- or microspheres, and/or a plurality of tubular nanostructures (e.g., fibrils, i.e., fibers, and tubes), each encapsulating an esterase or a functional fragment thereof, wherein each one of said nano- and/or microspheres, or said tubular nanostructures, being formed (i.e., made) of a plurality of aromatic dipeptides comprising end-capping modified aromatic dipeptides, non-modified aromatic dipeptides, or a combination thereof; and said composition having a pH suitable for the activity of said esterase. According to the invention, the end-capping modified aromatic dipeptides may be aromatic dipeptides protected at an either an amino- or carboxyl group thereof, e.g., at the N-terminus thereof; and the molar ratio between the end-capping modified- and the non-modified aromatic dipeptides, when a combination of both is present, may range from, e.g., about 1:1 to about 1:100. The compositions disclosed may further comprise ions of a metal capable of coordinating with, and which is required for enzymatic activity of, said esterase, e.g., Mn or Zn.

The esterase encapsulated within said nano- or microspheres and/or said nanostructures may be a carboxylic ester hydrolase (EC 3.1.1) such as a 1,4-lactonase (EC 3.1.1.25) and a quorum-quenching N-acyl-homoserine lactonase (EC 3.1.1.81); or a phosphoric triester hydrolase (EC 3.1.8) such as an aryldialkylphosphatase (EC 3.1.8.1).

In another aspect, the present invention provides a plant, or a part, organ or plant propagation material thereof, at least partly covered or coated with a composition as defined above, wherein said esterase is a carboxylic ester hydrolase such as a 1,4-lactonase and a quorum-quenching N-acyl-homoserine lactonase.

In yet another aspect, the present invention relates to a method for treating or preventing an infection of a bacterium in a plant or apart, organ or plant propagation material thereof, being infected by or susceptible to a bacterium secreting a lactone selected from N-(3-hydroxybutanoyl)-L-homoserine lactone (C4-HSL), N-(3-oxo-hexanoyl)-homoserine lactone (C6-oxo-HSL), N-[(3S)-tetrahydro-2-oxo-3-furanyl]octanamide (C8-oxo-HSL), and N-[(3S)-tetrahydro-furanyl]decanamide (C10-HSL), said method comprising applying on said plant or said part, organ or plant propagation material thereof, a composition as defined above, wherein said esterase is a carboxylic ester hydrolase such as a 1,4-lactonase and a quorum-quenching N-acyl-homoserine lactonase.

In still another aspect, the present invention relates to a method for decomposing/degrading an organophosphorus compound, e.g., a pesticide such as a phosphate type-, thiono type-, thiol type-, or dithiol type organophosphorus pesticide, from a media contaminated with said organophosphorus compound, said method comprising applying to said contaminated media a composition as defined above, wherein said esterase is a phosphoric triester hydrolase such as an aryldialkylphosphatase. The media treated by this method may be, e.g., a soil, a produce, or a water source.

### BRIEF DESCRIPTION OF DRAWINGS

FIGS. 1A-1C show the wild-type AHL-lactonase, PPH, activity. (1A) wtPPH lactonase activity (with 0.2 mM TBBL as substrate, [E]₀=0.5 μM) at temperatures ranging from 25-60° C. Error bars indicate standard deviation from 3 repeats. (1B) Lactonase activity assayed with N-(3-oxohexanoyl)-homoserine lactone, C6-oxo-HSL, and wtPPH ([E]₀=0.01 μM, at 25° C.). (1C) Non-inoculated pear fruits, the puncturing location is circled (left); pear fruits were inoculated with E. amylovora bacteria incubated with enzyme's buffer (center) and with E. amylovora culture previously incubated with 2 μM wtPPH (right).

FIGS. 2A-2C show the free enzymes activity in vitro and efficacy on P. communis Costia cultivar's blooming branches in growth chamber. (2A) PPH-G55V evolved stable mutant maintained its lactonase activity (E₀=0.1 μM of both enzymes, 0.2 mM TBBL, at 25° C.). (2B) The lactonase activity of PPH-G55V with TBBL measured as fused to MBP, and after cleavage with Factor Xa Protease (E₀=0.1 μM, 0.2 mM TBBL, at 25° C.). Data shown are average of 2 repeats. (2C) Inhibition of fire blight symptoms in Costia cultivar blooming branches in growth chamber by purified wtPPH and the PPH-G55V mutant. The results are presented as an average of inhibition rate±SE, with the * indicating significant differences from the infected plant without any treatment (only infection) (p<0.05, Tukey-Kramer HSD). The results show that treatment with 4 μM wtPPH or PPH-G55V, applied 2 h pre-infection, significantly inhibited the infection by 31.11% (p=0.0168) and 49.93% (p=0.0002), respectively, compared to the untreated control (only infection). No significant differences were seen between the wtPPH and the mutant variant (p>0.05).

FIGS. 3A-3D show that the mutant PPH-G55V increased the PPH residual activity at elevated temperatures, and that both the wtPPH and said mutant inhibited infection symptoms in blooming branches in a growth chamber. (3A) PPH-G55V evolved stabilized variant exhibiting 8° C. increase in T₅₀. Residual activity (%) presented as Average±std. (3B) Location of the mutated residues, G55V (blue) and H168Y (magenta), far from the active site, on the background of previously solved structure; PDB 4 if2. The conserved catalytic residues are shown as gray sticks, and the zinc metals ions shown as gray spheres. (3C) show a representative image of healthy pears flowers (upper); and a representative image of infected pears flowers 6 days after infection with (E. amylovora's cells per ml) E. amylovora. (lower). (3D) Fire blight symptoms inhibition±SE (%) in blooming branches of P. communis, Spadona sprayed either simultaneously with the bacterial culture (mix) or pre-infection. Infected blossoms with E. amylovora culture alone (only infection) were used as a control. As a standard positive control, 0.4 mg/ml Oxolinic acid pre-infection. * represents significant differences from the infection control group (LSD, p<0.05, n=30).

FIGS. 4A-4C show encapsulation of enzyme in BocFF spheres. (4A) Molecular structure of the BocFF peptide. (4B) Scanning electron microscopy micrograph of peptide nano-spheres. (4C) Confocal microscope images of labeled enzyme encapsulated in BocFF spheres with optical image (left), florescence image (center), and merged image showing the encapsulation of the labeled enzyme inside the spheres (right).

FIGS. 5A-5B show that encapsulated evolved mutant PPH-G55V has a long shelf life and reduces in planta fire-blight symptoms in the field. (5A) Enzyme encapsulation extended the shelf life of both wtPPH and PPH-G55V up to 37 days after purification and encapsulation. Residual activity presented as Average±Std (%). (5B) Blossoms of P. communis Spadona pear trees sprayed with free or encapsulated (labeled here as BocFF) PPH-G55V before infection (pre-infection), or by incubating the enzyme solution with a bacterial culture before simultaneous spraying. As a standard positive control, 0.4 mg/ml oxolinic acid was used pre-infection. Inhibition presents as Average±SE (%), * represents significant differences from the infected E. amylovora culture alone (only infection) (LSD, p<0.01, n=10).

FIGS. 6A-6B show that encapsulated BocFF-enzyme maintained lactonase activity and reduces infection in blooming branches of P. communis Costia cultivar in a growth chamber. (6A) The lactonase activity of the free enzyme, 0.25 μM PPH G55V, and following encapsulation with BocFF, was measured with 0.1 mM C6-oxo-HSL at room temperature. (6B) Average of infected flowers±SE (%) in each blossom, 6 days after infection with E. amylovora suspension mixed with encapsulated PPH-G55V (PPH-G55V BocFF, 4 μM), BocFF capsules and oxolinic acid (4 mg/L). ** indicate significant differences from BocFF capsules (p<0.01, Tukey-Kramer HSD, n=30-39). The results show that PPHG55V BocFF significantly reduced the percentages of infected flowers in a cluster to the level of 14.73% (p=0.0004), compared to 40.42% obtained with the empty BocFF. Oxolinic acid treatment reduced infection level to 10.17% (p>0.05).

FIG. 7 shows free enzyme inhibition of fire-blight symptoms caused by E. amylovora infection in P. communis Costia cultivar in the field. The results were inspected 34-days post inoculation. The results are presented as an average of inhibition rate±SE, * represents significant differences between treatments (p<0.05, each paired Student's t LSD n=10).

FIG. 8 shows the enzymatic activity of free MPH and MPH encapsulated in FmocFF hydrogel nanoparticles (HNPs).

FIG. 9 shows the enzymatic activity of free MPH and MPH encapsulated in BocFF; FF fibrils; and a mixture of BocFF and FF fibrils, over time in activity buffer.

FIG. 10 shows the encapsulation efficiency of MPH enzymes encapsulated in either FF or in a mixture of FF and BocFF fibers.

### DETAILED DESCRIPTION

It has now been found, in accordance with the present invention, that encapsulation of a lactonase, more specifically wild-type putative parathion hydrolase (wtPPH) or its mutant PPH-G55V, in nanospheres formed of BocFF peptides, extended substantially their shelf-life, i.e., enzyme stability over time, as measured by the enzyme reaction rate. The encapsulated enzymes displayed lactonase activity with AHLs secreted from the plant pathogen E. amylovora, with similar rate as the corresponding free enzymes. Surprisingly, the activity of the encapsulated wtPPH dropped by 50% only after 24 days, and the encapsulated evolved mutant, PPH-G55V, maintained full activity for 37 days. Moreover, treatment of infected blossoms of P. communis Spadona pear trees in the field with BocFF encapsulated PPH-G55V, prior to infection, inhibited significantly the infection symptoms relative to infected but untreated plants (control), resulting in 70% of pathogenicity inhibition. Similar inhibition degree was shown for the antibiotic oxolinic acid.

Interestingly, it has been further found that, in a similar manner, encapsulation of wild-type methyl parathion hydrolase (MPH) in fibrils made of BocFF peptides, FF peptides, or a combination of both peptides (FF-BocFF), increased the durability of the enzyme, with the highest increase at day 34 with FF-BocFF maintaining about 60% residual activity compared to 25% with the free enzyme.

In one aspect, the present invention provides a composition comprising a plurality of nano- or microspheres, and/or a plurality of tubular nanostructures (e.g., fibrils), herein also generally referred to “dipeptide-based structures”, each encapsulating an esterase or a functional fragment thereof, wherein each one of said nano- and/or microspheres, or said tubular nanostructures, being formed (i.e., made) of a plurality of aromatic dipeptides comprising end-capping modified aromatic dipeptides, non-modified aromatic dipeptides, or a combination thereof; and said composition having a pH suitable for the activity of said esterase.

In certain embodiments, the composition disclosed herein comprises a plurality of nano- and/or microspheres, each being formed of a plurality of aromatic dipeptides as defined above and encapsulating an esterase or a functional fragment thereof. In other embodiments, said composition comprises a plurality of tubular nanostructures (e.g., tubes or fibrils), i.e., spherical or elongated, preferably hollowed, tubular or conical structures having a diameter or a cross-section of preferably less than 1 μm, each being formed of a plurality of aromatic dipeptides as defined above and encapsulating an esterase or a functional fragment thereof. In further embodiments, the composition disclosed comprises a plurality of both nano- and/or microspheres, and tubular nanostructures, each being formed of a plurality of aromatic dipeptides as defined above and encapsulating an esterase or a functional fragment thereof.

The term “dipeptide” refers to a chain of two amino acid monomers (residues), linked by a peptide bond (amide bond), i.e., the covalent bond —C(O)NH— formed between two molecules, e.g., two amino acids, when a carboxyl group of one of the molecules reacts with an amino group of the other molecule, causing the release of a molecule of water.

The term “aromatic dipeptide”, as used herein, refers to any peptide consisting of two same or different amino acid residues, wherein at least one of said amino acid residues is an aromatic amino acid residue. Preferred aromatic dipeptides are those consisting of two same or different aromatic amino acid residues. The term “amino acid”, as used herein, refers to an organic compound comprising both amine and carboxylic acid functional groups, which may be either a natural or non-natural amino acid, and occur in both L and D isomeric forms. The twenty-two amino acids naturally occurring in proteins are aspartic acid (Asp), tyrosine (Tyr), leucine (Leu), tryptophan (Trp), arginine (Arg), valine (Val), glutamic acid (Glu), methionine (Met), phenylalanine (Phe), serine (Ser), alanine (Ala), glutamine (Gln), glycine (Gly), proline (Pro), threonine (Thr), asparagine (Asn), lysine (Lys), histidine (His), isoleucine (Ile), cysteine (Cys), selenocysteine (Sec), and pyrrolysine (Pyl). Non-limiting examples of other amino acids include citrulline (Cit), diaminopropionic acid (Dap), diaminobutyric acid (Dab), ornithine (Orn), aminoadipic acid, β-alanine, phenylglycine 1-naphthylalanine, 3-(1-naphthyl)alanine, 3-(2-naphthyl)alanine, γ-aminobutyric acid (GABA), 3-(aminomethyl)benzoic acid, p-ethynyl-phenylalanine, m-ethynyl-phenylalanine, p-chlorophenylalanine (4ClPhe), p-bromophenylalanine, p-iodophenylalanine, p-acetylphenylalanine, p-azidophenylalanine, p-propargly-oxy-phenylalanine, indanylglycine (Igl), (benzyl)cysteine, norleucine (Nle), azidonorleucine, 6-ethynyl-tryptophan, 5-ethynyl-tryptophan, 3-(6-chloroindolyl)alanine, 3-(6-bromoindolyl)alanine, 3-(5-bromoindolyl)alanine, azidohomoalanine, α-aminocaprylic acid, O-methyl-L-tyrosine, N-acetylgalactosamine-α-threonine, N-acetylgalactosamine-α-serine, and tetrahydro-isoquinoline-3-carboxylic acid (TIC). The term “amino acid residue” means a residue of an amino acid after removal of hydrogen atom from an amino group thereof, e.g., its α-amino group or side chain amino group if present, and/or —OH group from a carboxyl group thereof, e.g., its α-carboxyl group or side chain carboxyl group if present.

The term “aromatic amino acid residue”, as used herein, refers to an amino acid residue having an aromatic moiety for a side chain, such as a substituted or unsubstituted phenyl, a substituted or unsubstituted naphthalenyl, and a substituted or unsubstituted heteroaryl, e.g., indole, thiophene, imidazole, oxazole, thiazole, pyrazole, pyridine, pyrimidine, quinoline, isoquinoline, quinazoline, quinoxaline, and purine. When substituted, the phenyl, naphthalenyl or any other aromatic moiety includes one or more substituents such as, but not limited to, alkyl, trihaloalkyl, alkenyl, alkynyl, cycloalkyl, aryl, heteroaryl, halogen, —NO₂, azo, —OH, alkoxy, thiohydroxy, thioalkoxy, cyano, and amine. Non-limiting examples of substituted phenyls include pentafluoro phenyl, iodophenyl, biphenyl, and nitrophenyl.

The term “end-capping modified aromatic dipeptide”, as used herein, refers to a dipeptide which has been modified, e.g., protected, at the N-(amine) terminus and/or the C-(carboxyl) terminus thereof. End-capping protection refers to the attachment of a chemical moiety (also referred to herein as “protecting group/moiety”) to the terminus, so as to form a cap and thereby modify the terminus/termini of the peptide, i.e., the amine and/or carboxylic groups at the peptide's terminus. A chemical moiety attached to the N-terminus of a peptide is referred to as “N-terminus protecting group/moiety”; and a chemical moiety attached to the C-terminus of a peptide is referred to as “C-terminus protecting group/moiety”. The end-capping protection typically results in masking the charge of the peptide terminus, and/or altering chemical features thereof, such as, hydrophobicity, hydrophilicity, reactivity, or solubility. The end-capping moiety can be either aromatic or non-aromatic.

Examples of N-terminus protecting groups include, without being limited to, formyl, acetyl (also denoted herein as “Ac”), trifluoroacetyl, benzyl, benzyloxycarbonyl (Cbz), tert-butoxycarbonyl (Boc), trimethylsilyl (TMS), 2-trimethylsilyl-ethanesulfonyl (SES), trityl and substituted trityl groups, allyloxycarbonyl, 9-fluorenylmethyloxycarbonyl (Fmoc), and nitro-veratryloxycarbonyl (NVOC).

C-terminus protecting groups are typically moieties that lead to acylation of the carboxy group at the C-terminus, and examples of such groups include, without limiting, benzyl and trityl ethers, as well as alkyl ethers, tetrahydropyranyl ethers, trialkylsilyl ethers, allyl ethers, monomethoxytrityl, and dimethoxytrityl. Alternatively, the carboxy group at the C-terminus may be modified to an amide group.

Other end-capping modifications of peptides include replacement of the amine and/or carboxyl terminal groups by a different moiety, such as halogen, —OH, —SH, alkyl, aryl, alkoxy, thioalkoxy, aryloxy, thioaryloxy, and the like.

Additional examples of moieties suitable for peptide end-capping modification/protection can be found, e.g., in Green et al., “Protective groups in organic chemistry” (Wiley, 2^{nd} ed. 1991); and Harrison et al., “Compendium of synthetic organic methods”, vols. 1-8 (John Wiley and Sons, 1971-1996).

The term “non-modified aromatic dipeptide”, as used herein, means an aromatic dipeptide as defined above, featuring a free amine group at its N-terminus and a free carboxylic group at its C-terminus. A non-modified peptide is typically zwitterionic and features a neutral net charge, i.e., a neutral total charge when ionized in an aqueous solution at pH 7.

The term “halogen” as used herein refers to a halogen and includes fluoro, chloro, bromo, and iodo.

The term “alkyl” typically means a linear or branched hydrocarbyl group having, e.g., 1-18 carbon atoms and includes methyl, ethyl, n-propyl, isopropyl, n-butyl, sec-butyl, isobutyl, tert-butyl, n-pentyl, isoamyl, 2,2-dimethylpropyl, n-hexyl, n-heptyl, n-octyl, n-nonyl, n-decyl, n-undecyl, n-dodecyl, n-tridecyl, n-tetradecyl, n-pentadecyl, n-hexadecyl, and the like. Preferred are (C₁-C₈)alkyl groups, more preferably (C₁-C₄)alkyl groups, most preferably methyl, ethyl, and isopropyl. The alkyl group may optionally be substituted with one or more groups such as a trihaloalkyl (an alkyl substituted by three halogen atoms), alkenyl, alkynyl, cycloalkyl, aryl, heteroaryl, halogen, —NO₂, azo (—N═N—R, wherein R is alkyl or aryl), —OH, alkoxy, —SH, thioalkoxy, cyano (—CN), and amino (—NR′R″, wherein R′ and R″ each independently is H, alkyl, cycloalkyl, or aryl).

The terms “alkenyl” and “alkynyl” typically mean linear and branched hydrocarbyl groups having, e.g., 2-18 carbon atoms and one or more double or triple bond, respectively, and include ethenyl, propenyl, 3-buten-1-yl, 2-ethenylbutyl, 3-octen-1-yl, and the like, and propynyl, 2-butyn-1-yl, 3-pentyn-1-yl, and the like.

The term “cycloalkyl” means a mono- or bicyclic saturated hydrocarbyl group having, e.g., 3-10 carbon atoms such as cyclopropyl, cyclobutyl, cyclopentyl, cyclohexyl, cycloheptyl, adamantyl, and the like. The cycloalkyl may optionally be substituted with one or more groups such as alkyl, trihaloalkyl, alkenyl, alkynyl, cycloalkyl, aryl, heteroaryl, halogen, —NO₂, azo, —OH, alkoxy, thiohydroxy, thioalkoxy, cyano, and amino.

The term “alkoxy” refers to a group of the formula —O-alkyl or —O-cycloalkyl.

The term “thioalkoxy” refers to a group of the formula —S-alkyl or —S-cycloalkyl.

The term “aryl” denotes a univalent group derived from an aromatic carbocyclic group having, e.g., 6-14, carbon atoms consisting of a single ring or multiple rings either condensed or linked by a covalent bond such as, but not limited to, phenyl, naphthyl, phenanthryl, and biphenyl. The aryl may optionally be substituted with one or more groups such as alkyl, trihaloalkyl, alkenyl, alkynyl, cycloalkyl, aryl, heteroaryl, halogen, —NO₂, azo, —OH, alkoxy, thiohydroxy, thioalkoxy, cyano, and amino.

The term “heteroaryl” refers to a univalent group derived from a mono-, bi-, or poly-cyclic aromatic ring having, e.g., 4-12 atoms, and consisting of at least one carbon atom and at least one heteroatom selected from N, O, or S. Examples of mono-cyclic heteroaryls include, without being limited to, pyrrolyl, furyl, thienyl, thiazinyl, pyrazolyl, pyrazinyl, imidazolyl, oxazolyl, isoxazolyl, thiazolyl, isothiazolyl, pyridyl, pyrimidinyl, 1,2,3-triazinyl, 1,3,4-triazinyl, and 1,3,5-triazinyl. Polycyclic heteroaryl radicals are preferably composed of two rings such as, but not limited to, benzofuryl, isobenzofuryl, benzothienyl, indolyl, quinolinyl, isoquinolinyl, imidazo[1,2-a]pyridyl, benzimidazolyl, benzthiazolyl, benzoxazolyl, pyrido[1,2-a]pyrimidinyl and 1,3-benzodioxinyl. The heteroaryl may optionally be substituted by one or more groups such as alkyl, trihaloalkyl, alkenyl, alkynyl, cycloalkyl, aryl, heteroaryl, halogen, —NO₂, azo, —OH, alkoxy, thiohydroxy, thioalkoxy, cyano, and amino. It should be understood that when a polycyclic heteroaryl is substituted, the substitution may be in any of the carbocyclic and/or heterocyclic rings.

The term “aryloxy” refers to a group of the formula —O-aryl or —O-heteroaryl.

The term “thioaryloxy” refers to a group of the formula —S-aryl or —S-heteroaryl.

In certain embodiments, the composition of the present invention, according to any one of the embodiments above, comprises dipeptide-based structures, each being composed of, i.e., assembled from, a plurality of aromatic dipeptides comprising said end-capping modified aromatic dipeptides only. In particular such embodiments, each one of said end-capping modified aromatic dipeptides consists of two same or different aromatic amino acid residues and protected, e.g., at its N-terminus.

In other embodiments, the composition of the invention, according to any one of the embodiments above, comprises dipeptide-based structures, each being composed of, i.e., assembled from, a plurality of aromatic dipeptides comprising said non-modified aromatic dipeptides only. In particular such embodiments, each one of said non-modified aromatic dipeptides consists of two same or different aromatic amino acid residues.

In further embodiments, the composition disclosed herein, according to any one of the embodiments above, comprises dipeptide-based structures, each being composed of, i.e., co-assembled from, a plurality of aromatic dipeptides comprising a combination of said end-capping modified aromatic dipeptides and said non-modified aromatic dipeptides. The term “co-assembled”, as used herein, means that end-capping modified aromatic dipeptides are interlaced with non-modified aromatic dipeptides in the same structure, i.e., that at least a portion of the end-capping modified aromatic peptides are in interaction with at least a portion of the non-modified aromatic peptides within said dipeptide-based structures. Dipeptide-based structures co-assembled from both types of aromatic dipeptides are also referred to herein as “hybrid dipeptide-based structures”. The term “at least a portion” as used herein denotes at least 10%, at least 20%, at least 30%, at least 40%, at least 50%, at least 60%, at least 70%, at least 80%, at least 90%, at least 95%, at least 98%, or at least 99%. In particular such embodiments, each one of said end-capping modified aromatic dipeptides consists of two same or different aromatic amino acid residues and protected, e.g., at its N-terminus; and/or each one of said non-modified aromatic dipeptides consists of two same or different aromatic amino acid residues. In particular such embodiments, the molar ratio between said end-capping modified aromatic dipeptides and said non-modified aromatic dipeptides in said combination ranges from about 1:1 to about 1:100, e.g., from about 1:1 to about 1:90, from about 1:1 to about 1:80, from about 1:2 to about 1:70, from about 1:2 to about 1:60, from about 1:3 to about 1:50, from about 1:3 to about 1:40, from about 1:4 to about 1:30, from about 1:4 to about 1:20, or from about 1:5 to about 1:10, preferably from about 1:1 to about 1:50, respectively.

In certain embodiments, each one of the end-capping modified aromatic dipeptides composing the dipeptide-based structures comprised within the composition of the invention is an aromatic dipeptide protected at an either amino- or carboxyl group thereof with a non-aromatic- or aromatic group/moiety. In particular such embodiments, each one of said end-capping modified aromatic dipeptides is protected at the N-terminus thereof with, e.g., Boc or Fmoc.

In certain embodiments, each one of said non-modified aromatic dipeptides composing the dipeptide-based structures comprised within the composition of the invention is a homodipeptide such as phenylalanine-phenylalanine dipeptide, tyrosine-tyrosine dipeptide, and tryptophan-tryptophan dipeptide, preferably phenylalanine-phenylalanine dipeptide.

In other embodiments, each one of said end-capping modified aromatic dipeptides composing the dipeptide-based structures comprised within the composition of the invention is a homodipeptide such as phenylalanine-phenylalanine dipeptide, tyrosine-tyrosine dipeptide, and tryptophan-tryptophan dipeptide, preferably phenylalanine-phenylalanine dipeptide, protected at its either N- or C-terminus, but preferably at its N-terminus with, e.g., Boc or Fmoc. In particular such embodiments, each one of said end-capping modified aromatic dipeptides is an N-terminus modified-phenylalanine-phenylalanine dipeptide, e.g., phenylalanine-phenylalanine dipeptide protected at its N-terminu with Boc or Fmoc.

In further embodiments, each one of both the non-modified aromatic dipeptides and the end-capping modified aromatic dipeptides is a homedipeptide. In particular such embodiments, each one of said non-modified aromatic dipeptides is phenylalanine-phenylalanine dipeptide, tyrosine-tyrosine dipeptide, or tryptophan-tryptophan dipeptide, preferably phenylalanine-phenylalanine dipeptide; and each one of said end-capping modified aromatic dipeptides is phenylalanine-phenylalanine dipeptide, tyrosine-tyrosine dipeptide, or tryptophan-tryptophan dipeptide, preferably phenylalanine-phenylalanine dipeptide, protected at its either N- or C-terminus, but preferably at its N-terminus with, e.g., Boc or Fmoc.

In certain embodiments, the dipeptide-based structures comprised within the composition of the invention, at least in part, are nano- and/or microspheres as defined in any one of the embodiments above, characterized by a diameter in a range of from about 50 nm to about 2 micron, e.g., from about 100 nm to about 1500 nm, from about 200 nm to about 1000 nm, from about 300 nm to about 800 nm, or from about 400 nm to about 600 nm.

In certain embodiments, the dipeptide-based structures comprised within the composition of the invention, at least in part, are tubular, e.g., spherical or elongated, preferably hollowed, nanostructures as defined in any one of the embodiments above, are characterized by a length in a range of from about 10 nm to about 2000 nm, e.g., from about 20 nm to about 1800 nm, from about 30 nm to about 1600 nm, from about 40 nm to about 1400 nm, from about 50 nm to about 1200 nm, or from about 100 nm to about 1000 nm.

The composition of the invention comprises dipeptide-based structures according to any one of the embodiments above, each encapsulating an esterase or a functional fragment thereof, and said composition thus has a pH suitable for the activity of said esterase. In particular embodiments, the pH of the composition is within the range of from about 7 to about 11, e.g., from about 7.5 to about 10, from about 8 to about 9.5, or from about 8.5 to about 9, but preferably from about 7.5 to about 8.5.

In certain embodiments, the composition of the invention, according to any one of the embodiments above, further comprises ions of a metal capable of coordinating with, and which are required for enzymatic activity of, said esterase. In particular such embodiments, said ions are divalent ions of a metal selected from manganese (Mn), zinc (Zn), cobalt (Co), cadmium (Cd), iron (Fe), nickel (Ni), calcium (Ca), magnesium (Mg), and copper (Cu). In particular such embodiments, the composition disclosed comprises divalent ions of Mn or Zn.

Enzymes are generally classified by using the enzyme commission (EC) number, which is a numerical classification scheme for enzymes based on the chemical reactions they catalyze. As a system of enzyme nomenclature, every EC number is associated with a recommended name for the respective enzyme.

The composition of the invention comprises dipeptide-based structures according to any one of the embodiments above, each encapsulating an esterase (EC 3.1) or a functional fragment thereof.

The term “functional fragment”, as used herein with respect to the esterase encapsulated within the dipeptide-based structures composing the composition of the present invention, refers to a fragment of said esterase retaining the biological and enzymatic activity of said esterase, i.e., an amino acid sequence being a part of the amino acid sequence of the esterase and having biological and enzymatic activity substantially identical to those of the esterase.

In certain embodiments, the esterase encapsulated within the dipeptide-based structures is a carboxylic ester hydrolase (EC 3.1.1) such as a 1,4-lactonase (EC 3.1.1.25) and a quorum-quenching N-acyl-homoserine lactonase (EC 3.1.1.81), or a functional fragment thereof. In more particular such embodiments, said 1,4-lactonase is a phosphotriesterase like lactonase (PLL) or a functional fragment thereof, i.e., the composition disclosed comprises dipeptide-based structures according to any one of the embodiments above, each encapsulating PLL or a functional fragment thereof (also referred to herein as “PLL-based composition”).

In certain particular embodiments, the composition of the invention is a PLL-based composition, and the PLL encapsulated within the dipeptide-based structures is the wild-type putative parathion hydrolase (PPH) from M. tuberculosis (herein also identified as wtPPH) having the amino acid sequence of SEQ ID NO: 1. This enzyme is a quorum quenching enzyme (Afriat et al, 2006), capable of interfering with the quorum sensing signaling of various bacteria. Quorum sensing is a signaling system that occurs in various bacteria to sense its own population density and synchronize the expression of virulence genes via the secretion of small, diffusible signal molecules, such as N-acyl-homoserine lactone (AHL) (Jayaraman and Wood, 2008). These molecules play a critical role in triggering virulence gene expression in quorum sensing-dependent pathogens in plant pathogens, such as in the production of rotting enzyme (e.g., polygalacturonase) or biofilm components such as amylovoran (Vrancken et al., 2013).

In other particular embodiments, the composition of the invention is a PLL-based composition, and the PLL encapsulated within the dipeptide-based structures has a sequence comprising the amino acid sequence of SEQ ID NO: 1.

In further particular embodiments, the composition of the invention is a PLL-based composition, and the PLL encapsulated within the dipeptide-based structures is a mutant, variant or homolog of the wtPPH (SEQ ID NO: 1) that retains the biological and enzymatic activity of wtPPH, i.e., a mutant, variant or homolog, having at least 30% identity (homology) to wtPPH, and a TIM-barrel fold substantially identical to that of wtPPH; and preserving the catalytic residues of wtPPH, i.e., the six metal ligating residues in the active site: His26, His28, His182, His211, Asp268, and carbamoylated Lys149. Such compositions are also referred to herein interchangeably as “PLL homolog-based composition”, “PLL variant-based composition”, or “PLL mutant-based composition”.

The term “mutant”, “variant” or “homolog”, used herein interchangeably with respect to a specific esterase referred to, refers to any polypeptide having an amino acid sequence substantially identical to that of said esterase, in which one or more residues have been deleted and/or conservatively substituted with a functionally similar residue, and which retains the biological and enzymatic activities of said esterase. Examples of conservative substitutions include the substitution of a non-polar (hydrophobic) residue such as isoleucine, valine, leucine or methionine for another; the substitution of a polar (hydrophilic) residue for another, e.g., between arginine and lysine, between glutamine and asparagine, and between glycine and serine; the substitution of a basic residue such as lysine, arginine or histidine for another; or the substitution of an acidic residue such as aspartic acid or glutamic acid for another. Each possibility represents a separate embodiment of the present invention.

The term “TIM-barrel fold” is used herein in its conventional meaning and refers to a conserved protein fold, consisting of eight α-helices and eight parallel β-strands that alternate along the peptide backbone (Wierenga 2001).

Methods for determining tertiary structure of a protein or generating a model thereof are well-known in the arts and can easily be done for a large number of proteins. For example, a model of the TIM-barrel fold may be generated using MODPIPE, an automated software, pipeline, that calculates models on the basis of known structural templates and sequence-structure alignments (Pieper et al., 2014).

In some embodiments, the amino acid sequence having at least 30% identity, i.e., homology, to SEQ ID NO: 1 has 30%-99%, 30%-98%, 30%-97%, 30%-96%, 30%-95%, 30%-90%, 30%-85%, 30%-80%, 30%-75%, 30%-70%, 30%-65%, 30%-60%, 30%-55%, 30%-50%, 30%-45%, 30%-40%, 40%-99%, 40%-98%, 40%-97%, 40%-96%, 40%-95%, 40%-90%, 40%-85%, 40%-80%, 40%-75%, 40%-70%, 40%-65%, 40%-60%, 40%-55%, 40%-50%, 40%-45%, 50%-99%, 50%-98%, 50%-97%, 50%-96%, 50%-95%, 50%-90%, 50%-85%, 50%-80%, 50%-75%, 50%-70%, 50%-65%, 50%-60%, 50%-55%, 60%-99%, 60%-98%, 60%-97%, 60%-96%, 60%-95%, 60%-90%, 60%-85%, 60%-80%, 60%-75%, 60%-70%, 60%-65%, 70%-99%, 70%-98%, 70%-97%, 70%-96%, 70%-95%, 70%-90%, 70%-85%, 70%-80%, 70%-75%, 80%-99%, 80%-98%, 80%-97%, 80%-96%, 80%-95%, 80%-90%, 80%-85%, 90%-99%, 90%-98%, 90%-97%, 90%-96%, or 90%-95% identity with SEQ ID NO: 1.

In some embodiments, the amino acid sequence having at least 30% identity with SEQ ID NO: 1 has at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% identity with SEQ ID NO: 1.

Particular mutants, variants or homologs of wtPPH (SEQ ID NO: 1) are described, e.g., in WO 2020/255131, incorporated by reference as if fully disclosed herein.

In specific such embodiments, the composition of the invention is a PLL mutant-based composition, and the PLL encapsulated within the dipeptide-based structures is a mutant of wtPPH in which a glycine residue at a position corresponding to position 59 of SEQ ID NO: 1 is substituted by an amino acid residue selected from valine, alanine, leucine, and isoleucine; and/or a histidine residue at a position corresponding to position 172 of SEQ ID NO: 1 is substituted by an amino acid residue selected from tyrosine, phenylalanine and tryptophan, or a functional fragment thereof, wherein each one of the options represents a separate embodiment. In certain more specific such embodiments, said PLL is a mutant of wtPPH in which a glycine residue at a position corresponding to position 59 of SEQ ID NO: 1 is substituted by valine, e.g., a PLL comprising the amino acid sequence of SEQ ID NO: 2, or a functional fragment thereof. In other more specific such embodiments, said PLL is a mutant of wtPPH in which a histidine residue at a position corresponding to position 172 of SEQ ID NO: 1 is substituted by tyrosine, e.g., a PLL comprising the amino acid sequence of SEQ ID NO: 3, or a functional fragment thereof.

In this respect, it should be understood that the position, i.e., location, of a certain amino acid residue in a protein or fragment thereof disclosed herein is according to the numbering of the corresponding wild-type protein, and a substitution of an amino acid residue at a certain position with another amino acid residue is designated by referring to the one-letter code of the original amino acid residue, its position as defined above, and the one-letter code of the amino acid residue replacing the original amino acid residue. Thus, for example, the substitution of G59 in wtPPH (SEQ ID NO: 1) with valine would be designated G59V, and the substitution of H172 in said sequence with tyrosine would be designated H172Y.

Thus, for example, the glycine at the position corresponding to position 59 in the sequence of wtPPH, also referred to herein as G59, would be referred to as G59 also in a fragment of said PPH or in a homolog of said PPH, having different size according to alignment algorithms well known in the art of protein chemistry, such as Multiple Sequence Comparison by Log-Expectation (MUSCLE) or Multiple Alignment using Fast Fourier Transform (MAFFT). For clarity, the positions of the amino acid residues in the sequences of the fusion-proteins used in Examples 1-2 herein, G58 and H171, correspond to G59 and H172, respectively, in the isolated wild-type full length protein. Similarly, the sequence of the functionally active deletion mutant used to solve the three-dimensional structure of wtPPH lacks the four first N-terminal amino acid residues (Zhang et al., 2019). Consequently, glycine at position 55 in the enzyme characterized in Zhang et al., 2019 corresponds to G59 according to the system used to identify amino acid residue positions in the enzymes of the present invention.

In other specific such embodiments, the composition of the invention is a PLL mutant-based composition, and the PLL encapsulated within the dipeptide-based structures is a mutant of wtPPH comprising an amino acid sequence selected from the amino acid sequences of SEQ ID NOs: 4-6 (each having at least 30% homology to SEQ ID NO:1) and SEQ ID NOs: 7-101 (each having at least 79% homology to SEQ ID NO:1), or a functional fragment thereof.

The biological activity and/or enzymatic functioning of an enzyme, e.g., an esterase as referred to herein, are defined by substrate specificity and kinetic parameters such as k_{cat}, K_{M} and k_{cat}/K_{M}. Methods for measuring such an activity are well known in the art, and particular such methods for measuring a lactonase activity are exemplified in the Experimental section herein.

It appears that the lactonase activity of PPH on C6-oxo-HSL is one of the highest among native and engineered AHL degrading enzymes, as recently their specificity range was reviewed, indicating most enzyme degrade this AHL with k_{cat}/K_{M} values ranging from 10 to 10³ s⁻¹ M⁻¹ (Mandrich et al., 2010). Two enzymes with the same fold as PPH exhibited activity in the range of 10³ to 10⁴ s⁻¹ M⁻¹, and two enzymes with a different fold, more specifically AidC (N-acylhomoserine lactonase from the potato root-associated Chryseobacterium sp. strain StRB126 (Wang et al., 2012) and AHL-acylase from Delftia sp. (Maisuria and Nerurkar, 2015), exhibited higher activity at the range of 10⁵ s⁻¹ M⁻¹. None of these enzymes neither the naturally nor engineered are both highly active with C6-oxo-HSL and thermally stable enzyme. For example, an evolved mutant of a thermostable quorum quenching lactonase from the PLL family, exhibited very low activity with C6-oxo-HSL, with orders of magnitude lower specific activity (Chow et al., 2010).

Such enzymes have the potential to serve as environmentally safe, biodegradable, antibacterial treatments, however, agricultural use, requires the development of a delivery platform as well as stability and durability of enzymes. These requirements were addressed here using directed enzyme evolution and enzyme encapsulation of a highly active AHL lactonase. PPH-based point mutations library was screened for an increase in thermal resistance. As the coding gene of wtPPH was combined with the genes of improved mutants in the last round, the best identified mutant, after three rounds, contained a single point mutation, G55V, located at a distance from the active site. A recent review described how even for the very best characterized proteins, an understanding of the structure-function relationship is often limited to well-defined areas, typically substrate-binding sites, while the contribution of more remote amino acids, is generally poorly understood. Nevertheless, distant residues may still influence protein function, dynamics, expression, oligomerization, or stability (Wilding et al., 2019).

Following purifications, PPH-G55V mutant with increased thermal resistance, lost half of its activity after 13 days. In order to increase the enzymes shelf-life, the enzyme was encapsulated within BocFF nanospheres. The BocFF peptide spheres encapsulated enzymes presented increased durability, with extended shelf-life for both wtPPH and PPH-G55V in comparison to their free counterparts. To determine successful encapsulation of the enzymes within the spheres, the enzymes were fluorescently labeled. Confocal fluorescent microscopy images revealed that most of the fluorescent-labeled enzyme was located inside the BocFF spheres. Furthermore, the encapsulated enzyme displayed similar activity rate with a chromogenic lactone and C6-oxo-HSL secreted from the plant pathogen E. amylovora, as the free enzyme. Hence, it can be concluded that the peptide assembled capsules are permeable to the lactone substrates but not to the enzyme, and the enzyme within the capsules degrade the substrates following their penetration. Therefore, it can be suggested that these capsules can serve as enzymes protective microenvironment for an extended period of time.

In certain embodiments, the esterase encapsulated within the dipeptide-based structures is a phosphoric triester hydrolase (EC 3.1.8) such as an aryldialkylphosphatase (EC 3.1.8.1; also known as, e.g., phosphotriesterase), or a functional fragment thereof, i.e., the composition disclosed comprises dipeptide-based structures according to any one of the embodiments above, each encapsulating an aryldialkylphosphatase or a functional fragment thereof (also referred to herein as “aryldialkylphosphatase-based composition”).

In certain particular embodiments, the composition of the invention is an aryldialkylphosphatase-based composition, and the aryldialkylphosphatase encapsulated within the dipeptide-based structures is the wild-type methyl parathion hydrolase (MPH) from Pseudomonas sp. WBC-3, having the amino acid sequence of SEQ ID NO: 102 (herein also identified as wtMPH).

In other particular embodiments, the composition of the invention is an aryldialkylphosphatase-based composition, and the aryldialkylphosphatase encapsulated within the dipeptide-based structures has a sequence comprising the amino acid sequence of SEQ ID NO: 102.

In further particular embodiments, the composition of the invention is an aryldialkylphosphatase-based composition, and the aryldialkylphosphatase encapsulated within the dipeptide-based structures is a mutant, variant or homolog of wtMPH (SEQ ID NO: 102) that retains the biological and enzymatic activity of wtMPH, i.e., a mutant, variant or homolog, having at least 70% identity (homology) to wtMPH; and preserving the catalytic residues of said wild type MPH, i.e., the seven catalytic residues of said wild type MPH are His147, His149, Asp151, His152, His234, His302, and Asp255, in its active site. Such compositions are also referred to herein interchangeably as “aryldialkylphosphatase mutant-based composition”, “aryldialkylphosphatase variant-based composition”, or “aryldialkylphosphatase homolog-based composition”. In specific such embodiments, the aryldialkylphosphatase encapsulated within the dipeptide-based structures is a mutant of wtMPH selected from any one of the mutants described in the literature, e.g., in Ng et al., 2015; Cho et al., 2006; Cho et al., 2004; Lyagin and Efremenko, 2018; and Goldsmith et al., 2017.

In some embodiments, the amino acid sequence having at least 70% identity, i.e., homology, to SEQ ID NO: 102 has 70%-99%, 70%-98%, 70%-97%, 70%-96%, 70%-95%, 70%-90%, 70%-85%, 70%-80%, 70%-75%, 80%-99%, 80%-98%, 80%-97%, 80%-96%, 80%-95%, 80%-90%, 80%-85%, 90%-99%, 90%-98%, 90%-97%, 90%-96%, or 90%-95% identity with SEQ ID NO: 102.

In some embodiments, the amino acid sequence having at least 70% identity with SEQ ID NO: 102 has at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% identity with SEQ ID NO: 102.

In certain particular embodiments, the composition of the invention is an aryldialkylphosphatase-based composition, and the aryldialkylphosphatase encapsulated within the dipeptide-based structures is the phosphotriesterase (PTE) from Pseudomonas diminuta, having the amino acid sequence of SEQ ID NO: 103 (herein also identified as wtPTE). It should be noted that SEQ ID NO: 103, which represents the solved structure of said enzyme, starts with glycine at position 34 and the first amino acid in said sequence is thus referred to as G34.

In other particular embodiments, the composition of the invention is an aryldialkylphosphatase-based composition, and the aryldialkylphosphatase encapsulated within the dipeptide-based structures has a sequence comprising the amino acid sequence of SEQ ID NO: 103.

In further particular embodiments, the composition of the invention is an aryldialkylphosphatase-based composition, and the aryldialkylphosphatase encapsulated within the dipeptide-based structures is a mutant, variant or homolog of wtPTE (SEQ ID NO: 103) that retains the biological and enzymatic activity of wtPTE, i.e., a mutant, variant or homolog, having at least 70% identity (homology) to wtPTE; and preserving the catalytic residues of wtPTE, i.e., the six ligating residues His55, His57, His201, His230, Asp301, and carbamoylated Lys169, in its active site.

In some embodiments, the amino acid sequence having at least 70% identity, i.e., homology, to SEQ ID NO: 103 has 70%-99%, 70%-98%, 70%-97%, 70%-96%, 70%-95%, 70%-90%, 70%-85%, 70%-80%, 70%-75%, 80%-99%, 80%-98%, 80%-97%, 80%-96%, 80%-95%, 80%-90%, 80%-85%, 90%-99%, 90%-98%, 90%-97%, 90%-96%, or 90%-95% identity with SEQ ID NO: 103.

In some embodiments, the amino acid sequence having at least 70% identity with SEQ ID NO: 103 has at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% identity with SEQ ID NO: 103.

For practical purposes, the esterase encapsulated by the dipeptide-based structures referred to herein, according to any one of the embodiments above, may be provided as a fusion protein containing a tag useful for separating it from the cell extract by specific binding to a ligand-containing substrate, or for improving solubility.

For example, the esterase for encapsulation by the dipeptide-based structures referred to herein may be provided as a fusion protein with a maltose binding protein at the N-terminus. Other examples of tags include chitin binding protein (CBP), Strep-tag (e.g., the peptide AWRHPQFGG, which displays intrinsic binding affinity towards streptavidin), glutathione-S-transferase (GST), and poly(His) tag. Tags including thioredoxin (TRX) and poly(NANP), used to improve solubility of enzymes may also be used. The tag is optionally removable by chemical agents or by enzymatic means, such as proteolysis or intein splicing.

Alternatively, said esterase may be provided as a fusion protein containing a signal sequence facilitating its secretion into the growth medium. This is useful because it eliminates the need for disrupting the cells and provides for harvesting the protein of the invention simply by collecting the growth medium. The signal sequence is tailored for the host cell type used to express the protein. Freudl (2018) teaches that, in bacteria, two major export pathways, the general secretion or Sec pathway and the twin-arginine translocation or Tat pathway, exist for the transport of proteins across the plasma membrane. The routing into one of these alternative protein export systems requires the fusion of a Sec- or Tat-specific signal peptide to the amino-terminal end of the desired target protein.

The esterase for encapsulation by the dipeptide-based structures referred to herein may be provided as a fusion protein containing a Sec or Tat signal peptide. These peptides possess a similar tripartite overall structure consisting of a positively charged n-region, a central hydrophobic h-region, and a polar c-region that contains the recognition site (consensus: A-X-A) for signal peptidase (SPase; the cleavage site is indicated by an arrow). In Tat signal peptides, a characteristic amino acid consensus motif including two highly conserved arginine residues is present at the boundary between the often significantly longer n-region and the h-region. Furthermore, the h-region of Tat signal peptides is mostly less hydrophobic than those found in Sec signal peptides and in the c-region of Tat signal peptides, frequently positively charged amino acids (the so-called Sec-avoidance motif) are present that prevent a mistargeting of Tat substrates into the Sec pathway.

Since signal peptides, besides being required for the targeting to and membrane translocation by the respective protein translocases, also have additional influences on the biosynthesis, the folding kinetics, and the stability of the respective target proteins, it is not possible so far to predict in advance which signal peptide will perform best in the context of a given target protein and a given bacterial expression host. However, methods for finding an optimal signal peptide for a desired protein are well known and are described e.g., in Freudl (2018) (incorporated by reference as if fully disclosed herein). The signal sequence may be removed during the process of secretion or it is optionally removable by chemical agents or by enzymatic means, such as proteolysis or intein splicing.

Accordingly, any one of the mutants of wtPPH, wtMPH, or wtPTE referred to herein, when fused to a tag, may lack 1-10, e.g., 1-4, amino acid residues at its N- or C-terminus (as compared with wtPPH, wtMPH or wtPTE, respectively). Furthermore, a linker such as poly-asparagine of, e.g., about 10 residues, may be inserted between the sequence of the tag and the sequence of said mutant.

In certain embodiments, the present invention provides a composition as defined in any one of the embodiments above wherein the esterase encapsulated is a carboxylic ester hydrolase such as a 1,4-lactonase and a quorum-quenching N-acyl-homoserine lactonase, said composition further comprising a Tris buffer and divalent ions of Mn. In particular such embodiments, said Tris buffer is 50 mM Tris (at pH of about 8), and said Mn ions are provided as MnCl₂ at a concentration of about 100 μM.

In other embodiments, the present invention provides a composition as defined in any one of the embodiments above wherein the esterase encapsulated is a phosphoric triester hydrolase such as an aryldialkylphosphatase, said composition further comprising a Tris buffer and divalent ions of Zn. In particular such embodiments, said Tris buffer is 50 mM Tris (at pH of about 8), and said Zn ions are provided as ZnCl₂ at a concentration of about 100 μM.

Compositions as disclosed herein for use in agriculture, e.g., wherein the esterase encapsulated by the dipeptide-based structures is a carboxylic ester hydrolase such as a 1,4-lactonase and a quorum-quenching N-acyl-homoserine lactonase; or a phosphoric triester hydrolase such as an aryldialkylphosphatase, may further comprise an agriculturally acceptable surfactant such as a soap, higher alcohol sulfate, alkyl sulfonate, alkylaryl sulfonate, quaternary ammonium salts, polyalkylene oxide; a coating agent such as xanthan gum and talc, sodium lignosulfate, carboxymethylcellulose sodium and dextrin; a gel-forming agent such as sodium alginate; a wetting agent such as Genapol® X060—a fatty alcohol polyglycol ether and AF® 365 Antifoam—a polydimethylsiloxane antifoam emulsion; a non-ionic surfactant antifoam agent such as AF® 365 Antifoam—a polydimethylsiloxane antifoam emulsion; and/or a stabilizer such as glycerol. The composition may also comprise one or more solid carriers, liquid carriers, emulsifying and dispersing agents, etc., which are all well known in the art. Examples of these carriers include acacia, acidic terra abla, bentonite, calcium carbonate, carbon dioxide, clay, diatomaceous earth, freon, kaolin, nitrocellulose, and starch.

Particular compositions as disclosed herein, wherein the esterase encapsulated by the dipeptide-based structures is a carboxylic ester hydrolase such as a 1,4-lactonase and a quorum-quenching N-acyl-homoserine lactonase, may further comprise an additional antimicrobial agent such as, without limiting, a metal, e.g., silver or copper, or an alloy thereof (brass, bronze, cupronickel, copper-nickel-zinc); a metal ion salt, e.g., copper sulfate (CuSO₄); an antibiotic used in plant agriculture, e.g., streptomycin sulfate, oxytetracycline, oxolinic acid, and gentamicin; or a fungicide, e.g., mancozeb, tricyclazole, carbendazim, hexaconazole, metalaxyl, benomyl, difenoconazole, propiconazole, kitazin, tebuconazole, copper oxychloride, tridemorph, and propineb.

The compositions disclosed herein may be prepared utilizing any suitable method or technique, e.g., as described in detail in the Experimental section hereinafter. Depending on the specific aromatic dipeptides and the conditions used, such compositions may be obtained in the form of a liquid, e.g., a solution or suspension, hydrogel, or hydrogel nanoparticles (HNPs). For example, compositions as shown herein, prepared from FF peptides, BocFF peptides or a combination thereof, were obtained in the form of a solution or suspension containing either spheres or fibrils; and compositions prepared from FmocFF peptides were obtained in the form of either a hydrogel or HNPs (nanoparticles each in the form of a hydrogel), depending on the process conditions.

In certain embodiments, the composition of the present invention is thus liquid and formulated as a solution (for dissemination as, e.g., a spray). In other embodiments, the composition is semi-liquid and formulated as a hydrogel or hydrogel nanoparticles. In further embodiments, said composition is in dried form (lyophilized or freeze dried), i.e., formulated as a solid material (e.g., powder).

In another aspect, the present invention provides a plant, or a part, organ or plant propagation material thereof, at least partly covered or coated with a composition according to any one of the embodiments above, wherein said esterase is a carboxylic ester hydrolase such as a 1,4-lactonase and a quorum-quenching N-acyl-homoserine lactonase.

In certain embodiments, the plant is selected from Rosaceae crops such as apple and pear trees; carrot; potato; tomato; leafy greens; squash and other cucurbits; onion; green peppers; Gesneriacea such as African violets; beet; and potato.

Today more than ever, there is a demand for environmentally safe anti-bacterial treatments with low toxicity and short-term persistence against plant bacterial pathogens.

Quorum sensing has been shown to control genes that mediate bacterial pathogenicity, colonization of host surfaces, and adaptation to different environments, which makes the process interesting as an anti-microbial target. The application of enzymes degrading signaling molecules secreted by bacterial pathogens has been offered as a promising alternative.

As shown in the Experimental section hereinafter, following purifications, PPH-G55V lost half of its activity after 13 days. However, encapsulation of both wtPPH and PPH-G55V in BocFF peptide spheres increased durability, with substantially extended shelf-life for both enzymes in comparison to their free counterparts.

Specifically, treatment of pear trees, in the field, with both free and encapsulated mutant of the wtPPH (PPH-G55V), prior to infection with bacteria, significantly inhibited symptoms of fire blight with an efficacy similar to that of the antibiotic treatment with oxolinic acid, i.e., by up to 70%.

In yet another aspect, the present invention thus provides a method for treating or preventing a bacterial infection in a host infected by or susceptible to a bacterium causing disease through quorum sensing regulation systems, wherein said bacteria secret a lactone selected from N-(3-hydroxybutanoyl)-L-homoserine lactone (C4-HSL), N-(3-oxo-hexanoyl)-homoserine lactone (C6-oxo-HSL), N-[(3S)-tetrahydro-2-oxo-3-furanyl]octanamide (C8-oxo-HSL), and N-[(3S)-tetrahydro-furanyl]decanamide (C10-HSL), said method comprising applying or administering to said host a composition of any one of the above embodiments, wherein said esterase is a carboxylic ester hydrolase such as a 1,4-lactonase and a quorum-quenching N-acyl-homoserine lactonase. The substrate-specificity of wtPPH is known from Afriat et al., 2006.

Non-limiting examples of bacteria secreting one or more of the above-listed lactones include: (i) Pseudomonas aeruginosa, a Gram-negative opportunistic pathogen relying on a quorum sensing regulation system, is both a plant pathogen and a leading cause of morbidity and mortality in cystic fibrosis patients and immunocompromised individuals, secrets C4-HSL and C12-oxo-HSL) (Schuster and Greenberg, 2006); (ii) Pseudomonas fluorescens, can be found in soil and in water and is an important food spoiling bacteria secreting C8-HSL (Li et al., 2018). It is an unusual cause of disease in humans, and usually affects patients with compromised immune systems; (iii) Erwinia amylovora, causes fire blight on Rosaceae crops and produces and secretes either N-(3-oxo-hexanoyl)-homoserine lactone or N-(3-hydroxy-hexanoyl)-homoserine lactone (Venturi et al., 2004a); (iv) Pectobacterium carotovorum, causes bacterial stem rot and fruit rot in tomatoes and soft rot in potatoes and uses QS signaling to control the expression of pathogenicity factors, such as extracellular enzymes and the Hrp secretion system, and carbapenem antibiotic production (Crépin et al., 2012a; Crépin et al., 2012b; Bhat et al., 2010; Loh et al., 2002), which are mainly controlled by 3-oxo-C6 and 3-oxo-C8 AHL (Barnard and Salmond, 2007); (v) Pseudomonas corrugate, secrets N-hexanoyl-L-homoserine lactone (C6-AHL), C6-3-oxo-AHL and C8-AHL to regulate traits that contribute to virulence, antimicrobial activity and fitness (Licciardello et al., 2007); (vi) P. syringae, causes bacterial speck disease and is reported to use multiple QS circuits, specific to 3-oxo-C6, 3-oxo-C8 and C8-AHL (Lade et al., 2014); (vii) Burkholderia vietnamiensis, produces multiple AHL molecules, with the predominant AHL being N-decanoylhomoserine lactone (C10-HSL) and with C8-HSL and N-hexanoylhomoserine lactone (C6-HSL) (Conway and Greenberg, 2002); (viii) Burkholderia cepacia, secrets N-octanoylhomoserine lactone (C8-HSL) (Venturi et al., 2004b); and (ix) Burkholderia thailandensis, secrets N-oxo-decanoylhomoserine lactone (C10-oxo-HSL) and N-oxo-octanoylhomoserine lactone (C8-oxo-HSL) (Duerkop et al., 2009).

In certain embodiments, the host is a plant and the present invention thus provides a method for treating or preventing infection of a bacterium in a plant, or a part, organ or plant propagation material thereof, being infected by or susceptible to a bacterium secreting a lactone selected from C4-HSL, C6-oxo-HSL, C8-oxo-HSL, and C10-HSL, said method comprising applying on said plant or said part, organ or plant propagation material thereof, a composition according to any one of the embodiments above, wherein said esterase is a carboxylic ester hydrolase such as a 1,4-lactonase and a quorum-quenching N-acyl-homoserine lactonase.

Thus, in certain embodiments, the bacterium is selected from Erwinia amylovora, Pectobacterium carotovorum, Pseudomonas syringae, Pseudomonas corrugata, Burkholderia vietnamiensis, Burkholderia cepacia, Burkholderia thailandensis, and Pseudomonas aeruginosa, including any pathovars. In particular such embodiments, the bacterium is a bacterium secreting C6-oxo-HSL selected from Erwinia amylovora, Pectobacterium carotovorum, and Pseudomonas syringae.

In certain embodiments, the bacterium is Erwinia amylovora and the plant disease caused by it is fire blight on Rosaceae crops, e.g. pome fruit trees such as apple and pear.

In certain embodiments, the bacterium is Pectobacterium carotovorum and the plant disease caused by it, is bacterial soft rot on a plant such as carrot, potato, tomato, leafy greens, squash and other cucurbits, onion, green peppers, and African violets, and in particular beet vascular necrosis and blackleg of potato as well as slime flux on many different tree species.

In certain embodiments, the bacterium is Pseudomonas syringae and the plant disease caused by it is bacterial speck disease. In particular, the Pseudomonas syringae bacterium may be Pseudomonas tomato (formerly known as Pseudomonas syringae pv. tomato) and the disease tomato bacterial speck disease.

In certain embodiments, the host is a mammal, such as cystic fibrosis patients and immunocompromised individuals, and the bacterium is Pseudomonas aeruginosa or Pseudomonas fluorescens.

In certain embodiments, the method for treating or preventing an infection of a bacterium according to any one of the embodiments above, comprises applying a PLL-based composition of any one of the above embodiments. In certain particular embodiments, the method comprises applying a composition wherein the PLL encapsulated within the dipeptide-based structures is wtPPH having the amino acid sequence of SEQ ID NO: 1. In other particular embodiments, the method comprises applying a composition wherein the PLL encapsulated within the dipeptide-based structures has a sequence comprising the amino acid sequence of SEQ ID NO: 1. In further particular embodiments, the method comprises applying a composition wherein the PLL encapsulated within the dipeptide-based structures is a mutant, variant or homolog of wtPPH, e.g., a polypeptide having an amino acid sequence selected from SEQ ID NOS: 2-101, wherein each of these amino acid sequences represents a separate embodiment.

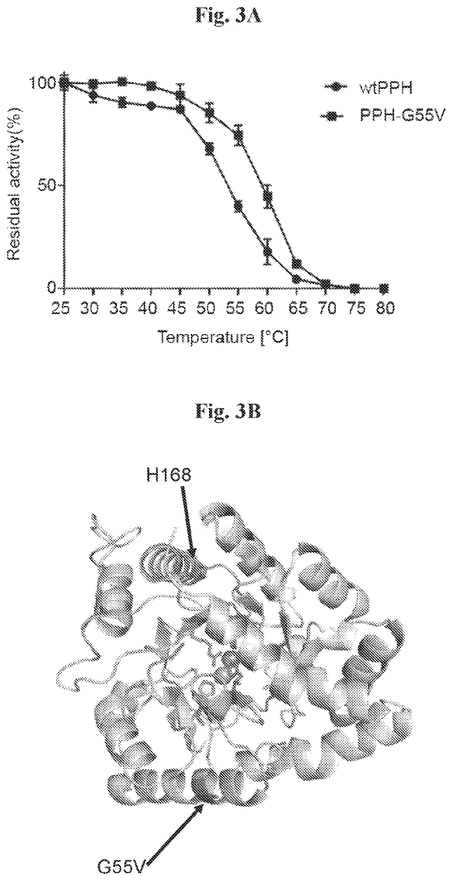

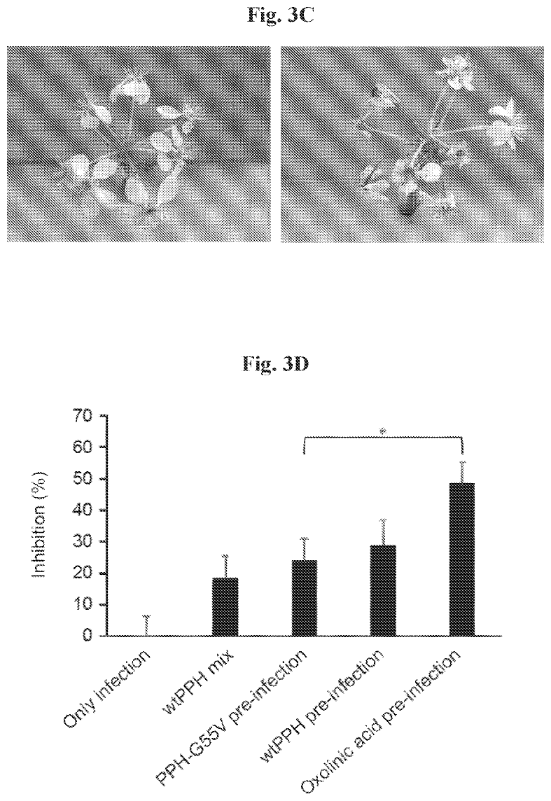

The term “treating” as used herein refers to means of obtaining a desired physiological effect. The effect may be therapeutic in terms of partially or completely curing a disease and/or symptom attributed to the disease. The term refers to inhibiting the disease, i.e., arresting its development; ameliorating the disease, i.e., causing regression of the disease; or protecting a plant, or a part, organ or plant propagation material thereof, from the disease by preventing or limiting infection. The term as used herein further refers to reduction of bacterial virulence as exhibited, e.g., in reduced extracellular polysaccharide (EPS) matrix or levan that contribute to the formation of the EPS (see FIG. 3 in WO 2020/255131).

The term “preventing” may be used herein interchangeably with the term “protecting” or “prophylactic treatment” and refers to application of the composition of the present invention to a susceptible host, i.e., mammal, or plant or a part, organ or plant propagation material thereof, before discernible microbial infection.

A method as disclosed hereinabove, when used for preventing an infection on, e.g., a seed, fruit, blossom or flower, may result in subsequent reduced infection as compared with a seed, fruit, blossom, or flower that was not subject to said method, and the term “prevention” should thus not be understood as necessarily resulting in the total absence of microbial infection or microbial presence, since the treatment neither kills the bacteria nor inhibits cell growth. The effect of such a method may be observed, e.g., in the case of seeds that have been subjected to the method prior to discernible infection, which subsequent to planting yield plants having higher stem length and foliage mass as compared to plants derived from seeds that have not been subject to this method. The difference in plant biomass yield is a result of the absence of infection, or reduced level of infection in the pretreated seeds that developed subsequent and in spite of the prophylactic treatment, as compared with the non-treated seeds. Powers, whole blossoms and fruit may similarly be pretreated by application of the composition of the present invention, which results in preservation of flower, blossom and fruit integrity and thus increased yield. Another example would be using said method for preventing infection of a microorganism in a plant or seedling growing in the vicinity of infected plants (from the same field or from other fields). In case the infective agent spreads from the infected plants or field to the initially non-infected plants or field, prophylactic treatment will protect the plants and thus result in higher yield as compared with plants or seedlings that have not been subject to this method.

The method for treating or preventing a bacterial infection as disclosed herein, comprises direct application of the composition to the plant or part, organ or plant propagation material thereof. The composition may be applied in a formulation such as granules, dusts, emulsifiable concentrates, wettable powders, pastes, water-based flowables, dry flowables, oil agents, aerosols, fogs or fumigants with suitable solid carriers, liquid carriers, emulsifying and dispersing agents, etc., as described above. In certain embodiments, the composition is applied to said plant or a part, organ or plant propagation material thereof, by spraying, immersing, dressing, coating, pelleting or soaking.

In certain embodiments, the plant propagation material treated by the method is a seed, root, fruit, tuber, bulb, rhizome, or part of a plant, wherein the composition is applied to the propagation material by spraying, immersing, dressing, coating, pelleting or soaking prior to or after detection of the infection.

In certain embodiments, the plant propagation material is a seed or a fruit. In other embodiments, the part of a plant is a leaf, branch, flower, blossom, inflorescence or a stem.

Synthetic organophosphorus compounds are used as pesticides, insecticides, plasticizers, air fuel ingredients, and chemical warfare agents. Contamination of soil from pesticides as a result of their bulk handling at the farmyard, or following application in the field or accidental release, may occasionally lead to contamination of surface and ground water. Several reports suggest that a wide range of water and terrestrial ecosystems may be contaminated with organophosphorus compounds. These compounds possess high mammalian toxicity and it is therefore essential to remove them from the environments.

In still another aspect, the present invention thus provides a method for decomposing/degrading an organophosphorus compound from a media contaminated with said organophosphorus compound, said method comprising applying to said contaminated media a composition according to any one of the embodiments above, wherein said esterase is a phosphoric triester hydrolase such as an aryldialkylphosphatase.

In certain embodiments, the media that is contaminated with the organophosphorus compound is a soil, a produce such as a fruit or vegetable, or a water source.

In certain embodiments, the organophosphorus compound being decomposed by the method disclosed hereinabove is a pesticide. Non-limiting examples of pesticide include phosphate type organophosphorus pesticides such as paraoxon, methyl paraoxon, and dichlorvos; thiono type organophosphorus pesticides such as parathion, methyl parathion, and chlorpyrifos; thiol type organophosphorus pesticides such as malathion and dimethoate; and dithiol type organophosphorus pesticides.

In certain embodiments, the method for decomposing/degrading an organophosphorus compound from a media contaminated with said organophosphorus compound according to any one of the embodiments above, comprises applying an aryldialkylphosphatase-based composition of any one of the above embodiments.

Compositions for use according to this method are preferably formulated as liquid, more specifically solutions, and application thereof may be carried out, e.g., by dispersing over the contaminated media, e.g., soil or produce, or introducing to the water source; or by immersing, dressing, coating, or soaking said media, e.g., produce, with said composition.

In certain particular embodiments, the method comprises applying a composition wherein the aryldialkylphosphatase encapsulated within the dipeptide-based structures is wtMPH having the amino acid sequence of SEQ ID NO: 102. In other particular embodiments, the method comprises applying a composition wherein the aryldialkylphosphatase encapsulated within the dipeptide-based structures has a sequence comprising the amino acid sequence of SEQ ID NO: 102. In further particular embodiments, the method comprises applying a composition wherein the aryldialkylphosphatase encapsulated within the dipeptide-based structures is a mutant, variant or homolog of wtMPH.

In yet other particular embodiments, the method comprises applying a composition wherein the aryldialkylphosphatase encapsulated within the dipeptide-based structures is wtPTE having the amino acid sequence of SEQ ID NO: 103. In still other particular embodiments, the method comprises applying a composition wherein the aryldialkylphosphatase encapsulated within the dipeptide-based structures has a sequence comprising the amino acid sequence of SEQ ID NO: 103. In further particular embodiments, the method comprises applying a composition wherein the aryldialkylphosphatase encapsulated within the dipeptide-based structures is a mutant, variant or homolog of wtPTE.

The term “decomposing/degrading an organophosphorus compound” as used herein refers to hydrolysis of said organophosphorus compound by a phosphoric triester hydrolase, acting on, e.g., esters of phosphonic and phosphinic acids, and phosphorus anhydrides, and consequently to hydrolysis/degradation of said compound.

The term “about” as used herein means that values which are 10% above or below the value provided are also included. Numbers that are not preceded by the term “about” are nevertheless to be understood as being modified in all instances by this term. Accordingly, unless indicated to the contrary, the numerical parameters set forth in this description and attached claims are approximations that may vary by up to plus or minus 10% depending upon the desired properties sought to be obtained by the present invention.

The invention will now be illustrated by the following non-limiting Examples.

### EXAMPLES

### Material and Methods

Cloning, expression, and purification. The synthetic gene encoding wild-type AHL-lactonase named PPH (wtPPH), abbreviation for its original annotation as Putative Parathion Hydrolase, from Mycobacterium tuberculosis, previously characterized (Afriat et al., 2006), was cloned into an expression vector, pMAL-c4X at its EcoRI and PstI sites (ordered from Syntezza). The pMAL-c4X vector was used for expression as a fusion protein with maltose binding protein (MBP), to give the construct pMAL-c4x-PPH, as it was previously described for PPH and other enzymes from the amidohydrolase superfamily, that their fusion to MBP increased the yield of the soluble, active enzyme, without altering the enzymatic parameters (Afriat et al., 2006; Roodveldt and Tawfik, 2005). Hereinafter, MBP-PPH will be referred to as PPH.

Expression and purification of AHL-lactonase (PPH) wild-type, variants thereof, and MPH. For large-scale production, the Luria-Bertani (LB) medium (5 mL) containing 100 μg/mL ampicillin and 0.5 mM MnCl₂ (for PPHs) or ZnCl₂ (for MPH) was inoculated with a single colony of E. coli-BL21 (DE3) cells, freshly transformed with pMAL-c4x-PPH/MPH, and grown overnight. The resulting culture was added to 500 mL of the same medium and grown overnight at 30° C. with shaking for about 5 h; when OD₆₀₀ reached values of 0.6-0.8, 0.4 mM isopropyl β-D-1-thiogalactopyranoside (IPTG) was added to induce expression. Following overnight incubation at 20° C., cells were harvested by centrifugation and resuspended in lysis buffer (50 mM Tris-HCl pH 8.0, 100 mM NaCl, 100 μM MnCl₂/ZnCl₂ and protease inhibitor Cocktail (Sigma) diluted 1:500). The subsequent steps were performed at 4° C. After centrifugation, the supernatants were passed through an amylose column (New England Biolabs, NEB) equilibrated with column buffer (50 mM Tris pH 8.0, 100 mM M NaCl, and 100 μM MnCl₂/ZnCl₂). The fusion proteins were eluted with column buffer supplemented with 10 mM maltose and loaded on a size-exclusion chromatography column, HiLoad 16/600 Superdex 75 pg column (GE Healthcare), adapted for the AKTA fast protein liquid chromatography (FPLC) system and equilibrated with filtered column buffer. The purity of the fusion enzymes was established by 12% sodium dodecyl sulfate-polyacrylamide gel electrophoresis (SDS-PAGE), and samples were stored at 4° C.

Enzyme kinetics analysis. For the determination of enzyme's biochemical parameters, the lactonase activity was analyzed by monitoring absorbance changes in 200 μL reaction volumes using 96-well plates and a microtiter plate reader (BioTeK, optical length of ˜0.5 cm) at 25° C. For each substrate, reactions were performed at the same concentration of organic solvent, regardless of substrate concentration. The hydrolysis of C6-oxo-homoserine lactone was monitored by following the appearance of the carboxylic acid products using a pH indicator as described previously (Afriat et al., 2006). 4-Thiobutyl-γ-butyrolactone (TBBL) was used as a proxy lactone substrate, as its thioester bond hydrolysis is easily detected with 0.5 mM 5,5′-dithiobis(2-nitrobenzoic acid, DTNB) as an indicator (Khersonsky and Tawfik, 2006), at 412 nm. The phosphotriesterase activity was analyzed using paraoxon and monitoring absorbance changes at 405 nm, as previously described (Afriat et al., 2006). Michaelis-Menten kinetics analysis was used to determine the catalytic parameters of wild-type PPH and its variants. The reaction mixtures contained 0.01-1 mM lactone substrates in 2.5 mM Bicine buffer, pH 8.3, and 0.2 M NaCl, supplemented with 0.2-0.3 mM cresol purple as a pH indicator (577 nm, ε=1550-2500 OD/M, 1% dimethyl sulfoxide (DMSO)). Initial rates (V₀) were corrected for the background rate of spontaneous hydrolysis in the absence of enzyme. Kinetic parameters were obtained by fitting initial rates directly to the Michaelis-Menten equation [V₀=k_{cat}[E]₀[S]₀/([S]₀+K_{M})] with GraphPad. Error ranges relate to the standard deviation of the data obtained from at least two independent measurements.

Library construction and screening. Genetic libraries based on the PPH were constructed using the GeneMorph II Random Mutagenesis Kit (Agilent), calibrated to produce an average of 2-3 non-synonymous mutations in the coding region of PPH in pMAL-c4X-PPH. Following the mutagenic polymerase chain reaction (PCR), libraries were cloned back into pMAL-c4X. The cloned libraries were then transformed into E. coli-BL21 (DE3) cells and plated on LB plates supplemented with 100 μg/ml ampicillin and 1% (w/v) glucose. In each round of screening, approximately 600 randomly chosen single colonies were picked and grown overnight at 37° C. with shaking, in 96 deep-well plates containing 500 μl of LB supplemented with 100 μg/mL ampicillin and 0.5 mM MnCl₂. The overnight cultures (at 1:20 dilution) were used to inoculate fresh LB medium (500 μl), supplemented with 100 μg/mL ampicillin and 0.5 mM MnCl₂ in 96 deep-well plates. Cells were grown at 30° C. with shaking for about 5 h, to an OD₆₀₀=0.6-0.8; then 0.4 mM IPTG was added to induce expression. Following overnight incubation at 20° C., the cells were pelleted and frozen at −80° C. Cells were suspended in a lysis buffer (50 mM Tris, pH 8, 100 μM MnCl₂, 100 mM NaCl, 100 μg/mL lysozyme, 0.5 unit/mL benzonase, 0.1% Triton X-100, 1:500 protease inhibitor cocktail (Sigma)) and incubated for 1 h at 25° C. with shaking at 960 rpm. The lysates were clarified by centrifugation, incubated at 50° C. for 30 minutes, cooled to room temperature, diluted in activity buffer, and assayed for hydrolysis of TBBL with 0.1 mM DTNB as an indicator. Three rounds of evolution were performed, in each round, to verify the rate of improvement; following streak plate isolation in LB plates, successful variants with more than two-fold improvement were overexpressed in triplicates (3 mL cultures) and lysed as described above, and following their incubation in 50° C., their lactonase activity (with TBBL) in lysates was measured at 25° C. Only the variants that were verified for improvements were selected for the next round.

Enzyme encapsulation in BocFF peptide nanospheres. Lyophilized Boc-Phe-Phe-OH peptide powder (Bachem, Switzerland) was dissolved in ethanol at a concentration of 50 mg/mL to form a stock solution. The stock solution was then diluted with buffer (50 mM Tris, pH 8, 100 mM NaCl, and 100 μM MnCl₂) and immediately diluted with the enzyme solution to obtain a peptide concentration of 4 mg/mL. The AHL lactonases, wild-type putative parathion hydrolase (wtPPH) and its mutants, or the phosphotriesterase wild-type methyl parathion hydrolase (wtMPH), were dissolved in the buffer solution and, upon self-assembly, encapsulated in the spheres. The compositions prepared by this process can be obtained in the form of a liquid, e.g., a solution or suspension.

Enzyme encapsulation in BocFF, FF peptide fibrils. Boc-Phe-Phe-OH, Phe-Phe-OH, or their co-assembly fibrils were made by dissolving the peptide powder in the buffer solution at a concentration of 4 mg/mL, at 100° C. for 30 min while stirring at 1500 rpm. Upon complete dissolution of the peptide, the solution was left to cool to 45° C. followed by the addition of the enzyme solution.

Enzyme encapsulation in FmocFF peptide hydrogel nanoparticles. Lyophilized Fmoc-Phe-Phe-OH peptide powder (Bachem, Switzerland) was dissolved in DMSO at a concentration of 100 mg/mL to form a stock solution. The peptide was then diluted with the buffer solution to 10 mg/mL and then homogenized for 30 min with a mineral oil solution containing 0.4% vitamin E. The solution was then continuously stirred at 4° C. at 1000 rpm for 2 hours. Upon completion, the hydrogel nanoparticles were washed with hexane and centrifuged.

Scanning electron microscopy (SEM) analysis. For SEM analysis, 10 μl of samples was placed on glass coverslips and then dried under a vacuum. The samples were then coated with a thin gold and viewed using SEM (JEOL, JSM-IT100 InTouchScope).

Confocal analysis of fluorescent labeling enzyme encapsulated in the peptide nanospherse. The enzymes were fluorescently labeled using Cy5-NHS-ester (Lumiprobe). Labeling was conducted by reacting 10 μM of enzyme with 30 μM reactive dye for 1 hour in an enzyme buffer followed by free Cy5-NHS-ester removal with a 10K molecular weight cut-off dialysis bags. The labeled enzymes were then encapsulated in BocFF nanospheres, and images were taken with a Leica SP8 X confocal microscope. Image J software was used to analyze and calculate the enzyme encapsulation efficiency.

Heat inactivation and shelf life. The heat inactivation assay was performed by pre-incubating purified PPH variants at temperatures ranging between 25-70° C. for 1 h, as previously described (Goldenzweig et al., 2016). Residual activity was then measured by monitoring lactonase activity with 0.2 mM TBBL for PPH or paraoxon for MPH, and 0.5 μM enzyme concentration at room temperature. The mid-point of temperature inactivation, the temperature at which 50% of the activity was retained (or lost) (T₅₀), was determined by fitting a two-state model using GraphPad. The shelf-life measurements of the lactonase activity of PPH variants (at 0.5 μM enzyme concentration) were performed with 0.1 mM TBBL for 34 days following purification while keeping the enzyme solution at room temperature. The shelf-life measurements of the paraoxonase activity of MPH (at 0.5 μM enzyme concentration) were performed with 0.1 mM paraoxon for 34 days following purification while keeping the enzyme solution at room temperature. The residual activity in percentage was calculated relative to the activity immediately following purification.

Pathogenicity in pears. To assess the ability of purified AHL lactonases (wild-type PPH and its mutant) to inhibit firelight diseases in pears, a single colony of E. amylovora (isolate 551) was cultured over-night in an LB medium (10 mL) and normalized to 10⁹ cfu (OD₆₀₀=0.5 corresponds to 10⁹ E. amylovora's cells/mL) and then pelleted and suspended in the enzyme activity buffer. Culture suspensions were incubated with either 0 or 2 μM purified enzymes (in 50 mM Tris, pH 8, and 100 μM MnCl₂) for 1 h at 28° C. and 300 rpm. Immature pears were sterilized with 70% ethanol and punctured with a sterile needle followed by inoculation with 10 μL of treated culture. The treated fruits were incubated in a humidified chamber at 28° C. for 10 days and monitored daily for disease symptoms, with pictures taken from the 7^{th} day. Each treatment consisted of 7-10 pears, and the experiment was repeated five times.

Pathogenicity assay in blooming branches in a growth chamber. Pathogenicity assays were performed based on an established protocol (Blachinsky et al., 2003) with minor changes: blooming branches with open flowers of Pyrus communis (P. communis), “Spadona” or “Costia”, were placed in a growth chamber at 22±1° C. (12 h photoperiod illuminated by cool-white fluorescent tubes). Enzyme solutions containing 4 μM of wild-type PPH and evolved mutants (in 50 mM Tris, pH 8, and 100 μM MnCl₂) were sprayed on the flowers. Two hours later, flowers were sprayed with a cell suspension (10⁷ E. amylovora's cells/mL) mixture (to increase bacterial infection) of two E. amylovora strains 511 (isolated from Yesud HaMa'ala, in 2009) and 576 (isolated from Merom Golan, in 2011), provided from the Israel Agriculture Research Organization (ARO) collection. Both strains are sensitive to oxolinic acid, a quinolone antibiotic commonly used for fire blight management. To improve the infection rate, inoculations were performed just before sunset, when ambient temperatures decreased and relative humidity increased. Alternatively, the enzyme and cultures were mixed in a 1:1 ratio and incubated for 30 minutes before spraying. E. amylovora mixed culture alone was used as a control. Treatment with 0.4 mg/mL oxolinic acid (Starner, produced by Sumitomo, Japan, and marketed in Israel by Adama Agan) was applied as a control treatment. Uninfected flowers were used as negative controls. The experiment was done in a randomized block design in three repeats, with 10 blossoms in each repeat. Closed flowers were removed. As no significant effect of the blocks was seen, we combined the repeats to give n>30. The air conditioning and the light in the chamber were shut off over night after infection, to preserve humidity. Fire blight symptoms were evaluated 3, 7, and 12 days after infection. The results after 7 days were calculated as the inhibition ratio:

$$Inhibition = \frac{( \% untreated ) - ( \% treated )}{( \% untreated )} \times 100$$

where % untreated is the percentage of infected flowers in only infected, untreated blossoms and % treated is the percentage of infected flowers in infected and treated blossoms. The only infected, untreated controls in P. communis Spadona and Costia cultivars had 82.0 and 50.26% infected flowers, respectively.

Pathogenicity in blossoms in the field. Blossoms of P. communis Spadona pear trees were sprayed with the evolved mutant PPH-G55V (4 μM) in the free or encapsulated state. Treatment was administered either 30-45 minutes before or concomitantly with the infection (by incubating the enzyme solution with the culture for 30 minutes before spraying). Treatment with 0.4 mg/mL oxolinic acid (Starner produced by Sumitomo, Japan, and marketed in Israel by Adama Agan), was applied as a control treatment. In all cases, bacterial culture of 10⁹ E. amylovora's cells/mL (mixture of isolates 511 and 576) were used. After infection, the blossoms were covered with a plastic bag overnight to ensure high humidity. The experiment was repeated five times with 10 blossoms in every repeat (5 on each side of the tree). Not more than four different treatments were conducted on a tree. As no significant effect of the tree location or to the tree side was seen (p>0.05), we combined the repeats to give n=10. Disease symptoms were evaluated 13 days post inoculation by counting the diseased flowers in each blossom, and again at 24 days post inoculation by counting infected blossoms. A similar experiment in P. communis Costia pears evaluated disease symptoms after 12 and 35 days post infection in the same manner. The field trials were conducted at the Hula Valley Orchards Experimental Farm in the north (33° 8′58.10″N 35° 37′16.93″E). The number of infected blossoms is shown for P. communis Spadona pear trees 24 days post inoculation. Inhibition ratios relative to untreated controls were calculated as described above. The infection rate in the “only infection” controls in P. communis Spadona and P. communis Costia were 56.67% and 62.0%, respectively.

Statistical analysis for the blossoms assay. Analysis of variance (ANOVA) was carried out using JMP 13 SAS Institute, 2016. The significance of the treatments was determined using student's (least significant difference, LSD) t-test or Tukey-Kramer honestly significant difference (HSD). Statistical effects of the blocks were evaluated for the studied parameters by ANOVA factorial design.

### Example 1. Wild-Type PPH from M. Tuberculosis can Proficiently Degrade the AHL Secreted from the Plant Pathogen E. amylovora and Reduce Infection in Planta

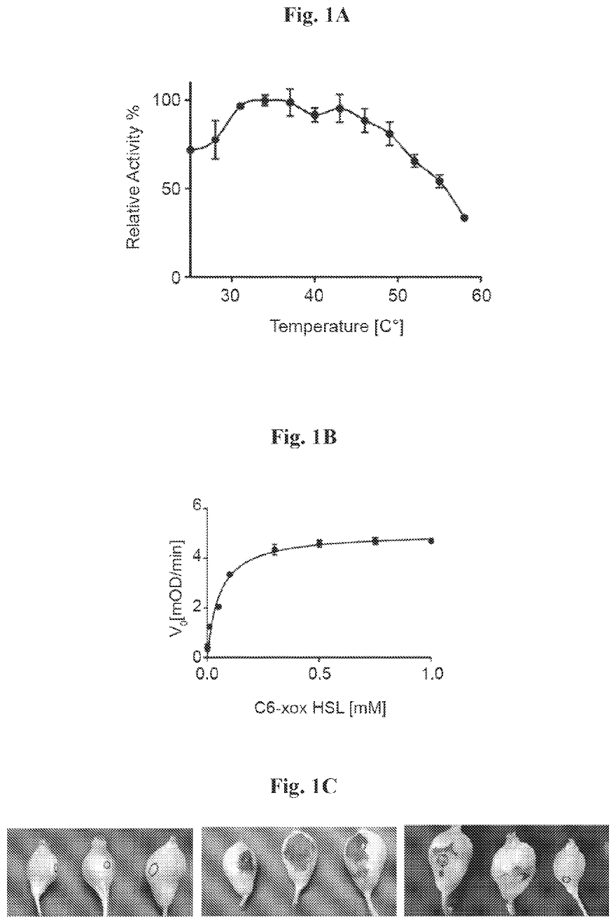

The wild-type PPH (wtPPH), abbreviation for its original annotation as putative parathion hydrolase, from M. tuberculosis, was previously characterized to be an efficient AHL lactonase belonging to the phosphotriesterase-like lactonase (Afriat et al., 2006). The phosphotriesterase-like lactonase belongs to the amidohydrolase superfamily, members of which possess a (β/α)₈ TIM-barrel fold (Holm and Sander, 1997), with a mono- or binuclear active-site metal center, with the conserved catalytic residues His 22, 24, 178, and 207, as well as Asp264. The sixth ligating residue is a carbamylated Lys145. The family comprises enzymes with diverse hydrolytic activities on very different substrates (Seibert and Raushel, 2005). We have previously characterized wtPPH with various AHLs (Afriat et al., 2006), and its highest catalytic activity was observed with oxo-medium chain AHLs, e.g., its k_{cat}/K_{M} value with C8-oxo-HSL was 0.55×10⁵ s⁻¹ M⁻¹. We surmised that its activity with C6-oxo-HSL, the AHL secreted from the plant pathogen E. amylovora, would be on the same order of magnitude. The synthetic gene encoding wtPPH was cloned into the expression vector, pMAL-c4X, and then overexpressed as the MBP-PPH fusion protein in E. coli-BL21 (DE3), as we previously saw that the fusion to MBP increased the yield of the soluble, active enzyme, without altering the enzymatic parameters (Afriat et al., 2006). Following cell lysis, the fusion protein was purified on an amylose column, followed by size exclusion chromatography. As E. amylovora is considered a psychotropic bacteria, since it grows at temperatures ranging from 4 to 37° C., with an optimum at 28° C. (Santander and Biosca, 2017), we examined the enzyme activity at various temperatures. The enzyme was found to be the most active at temperatures ranging from 34 to 38° C. and could maintain about 80% of its activity between 28 to 49° C. (FIG. 1A). The results of the pH indicator assay demonstrated high activity of the enzyme, with C6-oxo-HSL, indicating that wtPPH efficiently degrades it, with K_{M} value of 54.09±7.96 μM, a k_{cat} value of 5.61±0.16 s⁻¹, and k_{cat}/K_{M} of 1.04×10⁵ s⁻¹ M⁻¹ (E₀=0.01 μM, at 25° C.) (FIG. 1B). The ability of wtPPH to attenuate E. amylovora pathogenicity in planta was first evaluated in immature pears (FIG. 1C-E). Pear fruits were inoculated with E. amylovora cell suspension with enzymes buffer (50 mM Tris, pH 8, and 100 μM MnCl₂) or 2 μM wtPPH in buffer. The pears were incubated for 7 days at 28° C., the non-inoculated controls remained asymptomatic (FIG. 1C), and the pears inoculated with purified wtPPH plus E. amylovora appeared less symptomatic than pears inoculated with E. amylovora bacterial culture alone (FIG. 1D-E).

### Example 2. Isolating Improved Variants with Higher Thermal Resistance by Directed Enzyme Evolution

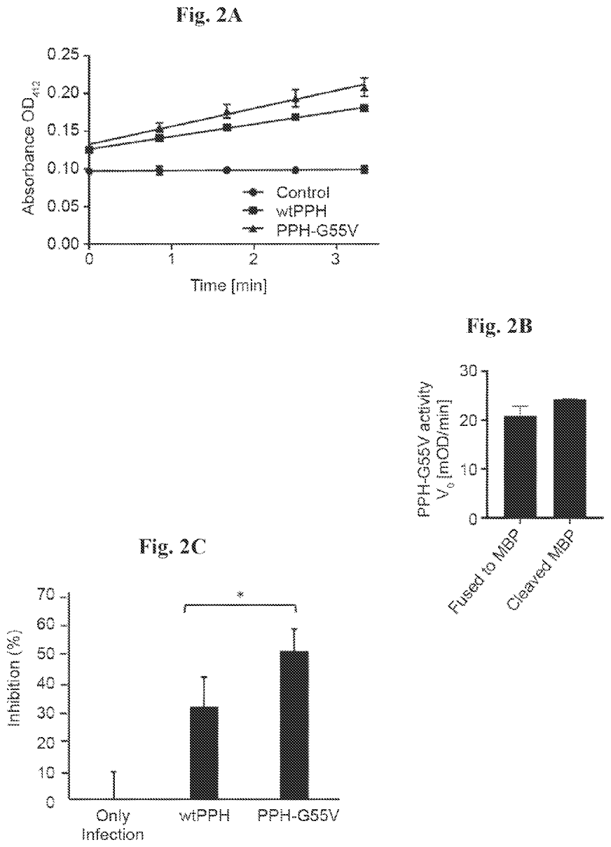

One of the main limitations with using enzymes for agriculture applications in the field is their loss of activity following exposure to elevated temperatures. As in the case of wtPPH, although it exhibited high catalytic activity of 1.04×10⁵ s⁻¹ M⁻¹, it lost activity following incubation at temperatures higher than 25° C., with 50% loss of activity above 50° C. (T₅₀), see FIG. 3A. For the directed evolution experiments of PPH towards increased temperature resistance, a library of about 3.4×10³ variants, with an error-rate of 2-3 nonsynonymous mutations per PPH coding gene, was constructed. Then, 600 variants of the transformed library were expressed in 96 deep-well plates and screened for an increase in the residual activity at 25° C., following the lysates incubation at 50° C. (the T₅₀ value for wtPPH). An improved mutant, harboring the mutation H168Y, was isolated in the first round, with about twofold increase in activity compared to wtPPH in lysates following lysate incubation at 50° C., and used to generate another random mutagenesis library. Since no better variant was detected in the second round, we generated another library where both plasmids with the coding genes of wtPPH and PPH-H168Y served as templates for mutagenesis. The third round of screening identified a variant with more than twofold increase in activity compared to wtPPH in lysates following lysate incubation at 50° C., and sequencing reveled it has one point mutation, G55V. We overexpressed the proteins on a large scale, purified as described in the Materials and Methods section, and verified that the mutant's lactonase activity was maintained; see the activity of wtPPH and PPH-G55V mutants with TBBL (E₀=0.1 μM of both enzymes, 0.2 mM TBBL, at 25° C., in FIG. 2A). We verified the fusion to MBP had no effect on the lactonase activity of PPH-G55V, following cleavage with a specific protease (Factor Xa), the lactonase activity was the same, see FIG. 2B. Next, we performed heat inactivation assay for wtPPH and its improved mutant PPH-G55V, and as FIG. 3A indicates, variant PPH-G55V displayed increased thermal resistance compared to wtPPH. At 54° C., wtPPH demonstrated 50% activity loss (T₅₀), while the T₅₀ of PPH-G55V was at about 62° C. Interestingly, both mutations, H168Y and G55V, identified separately in the evolved variants are located far from the active site: mutation G55 is located on α-helix 3, while residue H168 is on α-helix 7 (FIG. 3B).

Next, the ability of the wtPPH and its evolved mutant, PPH-G55V, to inhibit pathogenicity in blossoms of Spadona (FIGS. 3C-3D) and Costia (FIG. 2C) cultivars of P. communis in a growth chamber was evaluated. FIG. 3D presents the results of the inhibition degree of fire blight symptoms, 7 days post inoculation. As can be seen, prior treatment of the plants with the antibiotics oxolinic acid inhibited infection by 48.22%, and treatment with 4 μM of purified wtPPH or PPH-G55V before infection with the pathogen significantly inhibited the symptoms by 28.73% (p=0.0042) and 23.79% (p=0.0171) relative to the infected and untreated blossoms (only infection), respectively. When the enzymes were mixed with the bacterial cultures for 30 min before infection and then sprayed simultaneously, 18.26% inhibition was exhibited, which was not significantly different from untreated infected plants (p>0.05) served as the control. The inhibition of infection by treatment of blooming branches with purified enzymes was validated by an additional experiment (FIG. 2C).

### Example 3. Encapsulation of Purified Lactonases in Self-Assembled Peptide-Based Nanostructures Increases the Enzyme's Shelf Life and Reduces Pathogenicity in Planta in Growth Chamber and in the Field

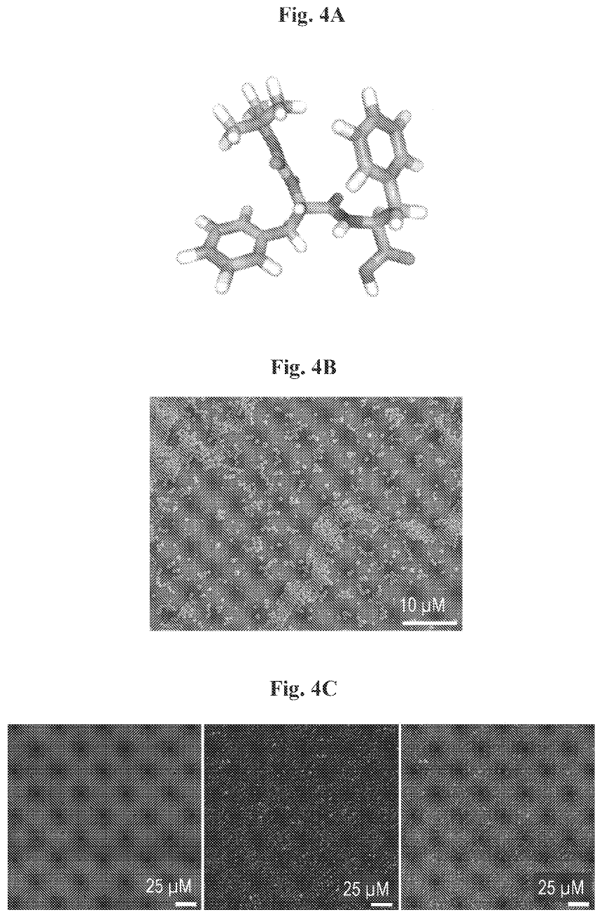

To improve the enzymes shelf life, the enzymes were encapsulated in peptide spheres. To form the encapsulated-nanospheres, BocFF peptide self-assembled in a solution containing the purified enzyme (FIGS. 4A-4B). To verify the encapsulation of the enzyme within the spheres, PPH-G55V was labeled with Cy5 prior to encapsulation and the florescence was examined with confocal microscopy. The results presented in FIG. 4C demonstrate Cy5-labeled-enzyme presence within the nanospheres, indicating successful encapsulation of the enzyme, as most of the Cy5-labeled-enzyme were located inside the BocFF spheres. Moreover, enzyme encapsulation efficiency was estimated via analysis of the confocal microscopy images, approximately 77±9%, showcasing the high encapsulation efficiency of the nanospheres.

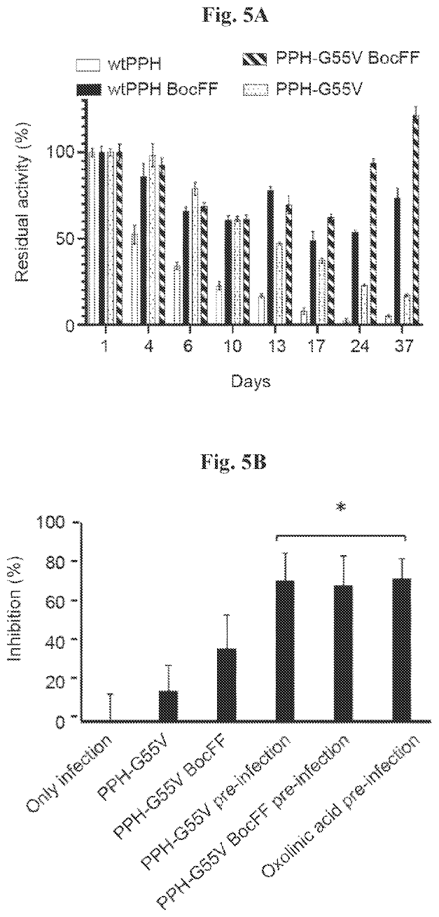

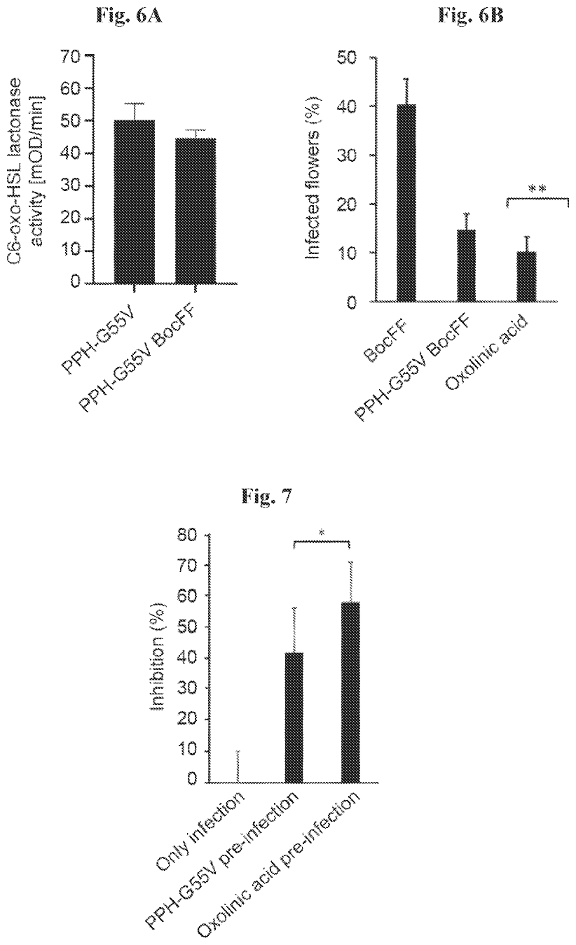

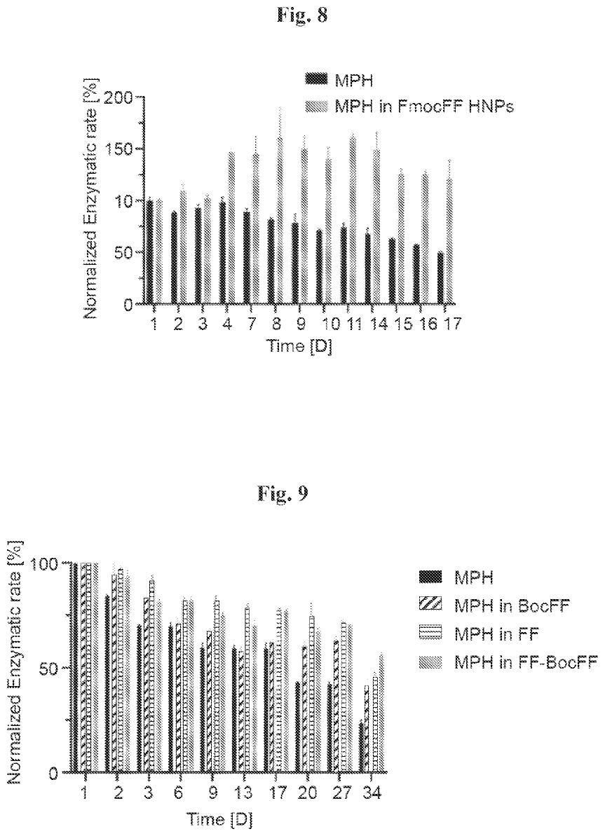

The shelf life, which monitors stability over time of the free and encapsulated enzyme, was assayed by measuring the reaction rate with the chromogenic lactone TBBL. It was observed that from the time of their purification, the free enzymes lost activity; however, the mutant was more stable over time than the wild-type enzyme. As presented in FIG. 5, the activity of wtPPH was reduced by 50% after 4 days and more than 95% after 37 days. In contrast, the mutant PPH-G55V lost 50% activity only after 13 days. Interestingly, BocFF encapsulation extended the shelf life of both wtPPH and PPH-G55V. The encapsulated enzymes displayed lactonase activity with TBBL and C6-oxo-HSL, the AHL secreted from the plant pathogen E. amylovora, with a similar rate as the free enzymes, see FIG. 6A for the activity with C6-oxo-HSL. The activity of encapsulated wtPPH dropped by 50% only after 24 days, and for the encapsulated evolved mutant, PPH-G55V maintained full activity for 37 days. Interestingly, on day 37, the residual activity values of BocFF-encapsulated wtPPH and PPH-G55V was 73 and 121%, respectively. The later exhibiting higher residual enzymatic activity than from the day of preparation can be explained by an increase in capsules permeability, as we observed a reduction in the range of 5-20% in enzyme activity following encapsulation; see FIG. 6A.

Next, the effect of the encapsulated BocFF-enzyme on infected detached blooming branches in growth chamber was tested. Notably, PPH-G55V encapsulated in BocFF significantly reduced the percentages of infected flowers in the cluster to 14.73%, similar to 0.4 mg/mL oxolinic acid treatment (p<0.05), and no inhibition of bacterial infection in blossoms following the spraying of empty capsules of BocFF was noticed (FIG. 6B). We then tested the ability of the free and encapsulated enzymes to inhibit pathogenicity in infected blossoms of Spadona cv. in the field. Following simultaneous application of the enzymatic treatments with the infecting bacteria, the infection symptoms were not inhibited significantly (p>0.05), demonstrating low and insignificant inhibition rates of 15.30% for the free mutant (PPH-G55V), and 36.47% for the BocFF-encapsulated mutant (PPH-G55V BocFF), relative to the control group, which were infected but untreated plants (only infection). However, when infected blossoms were treated with the purified enzymes before the bacterial infection (pre-infection), blossoms were significantly protected relative to untreated control and reaching inhibition level of 70.88% (p=0.0008) with the free PPH-G55V and 66.24% (p=0.0012) with the encapsulated PPH-G55V (PPH-G55V-BocFF) (FIG. 5B). This degree of inhibition is similar to that obtained with 0.4 g/L oxolinic acid, the antibiotic used routinely (71.77% inhibition of symptoms, p=0.0007). Similar results seen in a separate experiment performed with another P. communis cultivar, Costia, in the field (FIG. 7).

### Example 4. Encapsulation of MPH in FF Fibrils Exhibit a Protective Effect on the Enzyme by Increasing its Shelf-Life/Durability

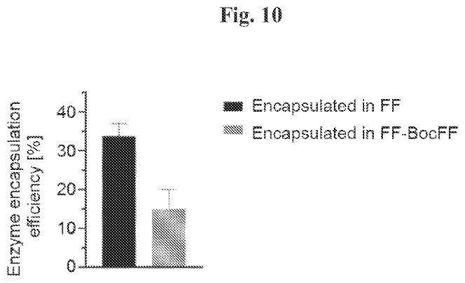

The use of pesticides for the control of insect populations has led to increased agricultural yield and reduced the spread of diseases caused by insects (Cooper and Dobson, 2007). However, the extensive use of pesticides, especially organophosphate-based pesticides, raised the community concern as their toxicity is not limited to insects and these chemicals can have serious or lethal effects on human health (Lyagin and Efremenko, 2018). Organophosphate-based pesticides irreversibly inhibit the enzyme acetylcholinesterase and interfere with the normal transmission of nervous signals. These compounds can bind other proteins, receptors and lead to various dysfunctions of cellular regulation, oxidative stress and apoptosis (Singh et al., 2020; Tallat et al., 2020). One of the promising ways to prevent these effects is by using bio-catalytic detoxification by various enzymes, such as MPH isolated from the soil bacteria Pseudomonas sp. WPC-3. Additionally, these bacteria can use methyl parathion as a sole carbon source. It was shown that MPH is a Zn²⁺ dependent hydrolase, efficient in hydrolyzing the organophosphate pesticide methyl-parathion. It was also shown that the enzyme is a dimer, and one of the Zn ions can be replaced by Cd²⁺ (Dong et al, 2005). To improve the enzyme shelf life, purified MPH was encapsulated in peptide spheres or fibrils, and the enzyme activity was tested with paraoxon at 405 nm over time. As shown, the enzyme encapsulation in all 3 peptides combinations increased durability, with highest increasement at day 34 with FF-BocFF maintaining about 60% residual activity compared to 25% with the free enzyme (FIG. 9). FIG. 10 shows that the encapsulation yield was about 40%.

APPENDIX
Sequence Listing

<SEQ ID NO: 1; PRT; Mycobacterium tuberculosis>
MISEFPELNTARGPIDTADLGVTLMHEHVFIMTTEIAQNYPEAWGDEDKRVAGAIA
RLGELKARGVDTIVDLTVIGLGRYIPRIARVAAATELNIVVATGLYTYNDVPFYFH
YLGPGAQLDGPEIMTDMFVRDIEHGIADTGIKAGILKCATDEPGLTPGVERVLRAV
AQAHKRTGAPISTHTHAGLRRGLDQQRIFAEEGVDLSRVVIGHCGDSTDVGYLEE
LIAAGSYLGMDRFGVDVISPFQDRVNIVARMCERGHADKMVLSHDACCYFDALP
EELVPVAMPNWHYLHIHNDVIPALKQHGVTDEQLHTMLVDNPRRIFERQGGYQ
<SEQ ID NO: 2; PRT; artificial sequence>
MISEFPELNTARGPIDTADLGVTLMHEHVFIMTTEIAQNYPEAWGDEDKRVAGAIA
RLVELKARGVDTIVDLTVIGLGRYIPRIARVAAATELNIVVATGLYTYNDVPFYFH
YLGPGAQLDGPEIMTDMFVRDIEHGIADTGIKAGILKCATDEPGLTPGVERVLRAV
AQAHKRTGAPISTHTHAGLRRGLDQQRIFAEEGVDLSRVVIGHCGDSTDVGYLEE
LIAAGSYLGMDRFGVDVISPFQDRVNIVARMCERGHADKMVLSHDACCYFDALP
EELVPVAMPNWHYLHIHNDVIPALKQHGVTDEQLHTMLVDNPRRIFERQGGYQ
<SEQ ID NO: 3; PRT; artificial sequence>
MISEFPELNTARGPIDTADLGVTLMHEHVFIMTTEIAQNYPEAWGDEDKRVAGAIA
RLGELKARGVDTIVDLTVIGLGRYIPRIARVAAATELNIVVATGLYTYNDVPFYFH
YLGPGAQLDGPEIMTDMFVRDIEHGIADTGIKAGILKCATDEPGLTPGVERVLRAV
AQAYKRTGAPISTHTHAGLRRGLDQQRIFAEEGVDLSRVVIGHCGDSTDVGYLEE
LIAAGSYLGMDRFGVDVISPFQDRVNIVARMCERGHADKMVLSHDACCYFDALP
EELVPVAMPNWHYLHIHNDVIPALKQHGVTDEQLHTMLVDNPRRIFERQGGYQ
<SEQ ID NO: 4; PRT; artificial sequence>
MKIEEGKLVIWINGDKGYNGLAEVGKKFEKDTGIKVTVEHPDKLEEKFPQVAATG
DGPDIIFWAHDRFGGYAQSGLLAEITPDKAFQDKLYPFTWDAVRYNGKLIAYPIAV
EALSLIYNKDLLPNPPKTWEEIPALDKELKAKGKSALMFNLQEPYFTWPLIAADGG
YAFKYENGKYDIKDVGVDNAGAKAGLTFLVDLIKNKHMNADTDYSIAEAAFNKG
ETAMTINGPWAWSNIDTSKVNYGVTVLPTFKGQPSKPFVGVLSAGINAASPNKEL
AKEFLENYLLTDEGLEAVNKDKPLGAVALKSYEEELAKDPRIAATMENAQKGEIM
PNIPQMSAFWYAVRTAVINAASGRQTVDEALKDAQTNSSSNNNNNNNNNNLGIE
GRISEFPELNTARGPIDTADLGVTLMHEHVFIMTTEIAQNYPEAWGDEDKRVAGAI
ARLGELKARGVDTIVDLTVIGLGRYIPRIARVAAATELNIVVATGLYTYNDVPFYF
HYLGPGAQLDGPEIMTDMFVRDIEHGIADTGIKAGILKCATDEPGLTPGVERVLRA
VAQAHKRTGAPISTHTHAGLRRGLDQQRIFAEEGVDLSRVVIGHCGDSTDVGYLE
ELIAAGSYLGMDRFGVDVISPFQDRVNIVARMCERGHADKMVLSHDACCYFDAL
PEELVPVAMPNWHYLHIHNDVIPALKQHGVTDEQLHTMLVDNPRRIFERQGGYQ
<SEQ ID NO: 5; PRT; artificial sequence>
MKIEEGKLVIWINGDKGYNGLAEVGKKFEKDTGIKVTVEHPDKLEEKFPQVAATG
DGPDIIFWAHDRFGGYAQSGLLAEITPDKAFQDKLYPFTWDAVRYNGKLIAYPIAV
EALSLIYNKDLLPNPPKTWEEIPALDKELKAKGKSALMFNLQEPYFTWPLIAADGG
YAFKYENGKYDIKDVGVDNAGAKAGLTFLVDLIKNKHMNADTDYSIAEAAFNKG
ETAMTINGPWAWSNIDTSKVNYGVTVLPTFKGQPSKPFVGVLSAGINAASPNKEL
AKEFLENYLLTDEGLEAVNKDKPLGAVALKSYEEELAKDPRIAATMENAQKGEIM
PNIPQMSAFWYAVRTAVINAASGRQTVDEALKDAQTNSSSNNNNNNNNNNLGIE
GRISEFPELNTARGPIDTADLGVTLMHEHVFIMTTEIAQNYPEAWGDEDKRVAGAI
ARLVELKARGVDTIVDLTVIGLGRYIPRIARVAAATELNIVVATGLYTYNDVPFYF
HYLGPGAQLDGPEIMTDMFVRDIEHGIADTGIKAGILKCATDEPGLTPGVERVLRA
VAQAHKRTGAPISTHTHAGLRRGLDQQRIFAEEGVDLSRVVIGHCGDSTDVGYLE
ELIAAGSYLGMDRFGVDVISPFQDRVNIVARMCERGHADKMVLSHDACCYFDAL
PEELVPVAMPNWHYLHIHNDVIPALKQHGVTDEQLHTMLVDNPRRIFERQGGYQ
<SEQ ID NO: 6; PRT; artificial sequence>
MKIEEGKLVIWINGDKGYNGLAEVGKKFEKDTGIKVTVEHPDKLEEKFPQVAATG
DGPDIIFWAHDRFGGYAQSGLLAEITPDKAFQDKLYPFTWDAVRYNGKLIAYPIAV
EALSLIYNKDLLPNPPKTWEEIPALDKELKAKGKSALMFNLQEPYFTWPLIAADGG
YAFKYENGKYDIKDVGVDNAGAKAGLTFLVDLIKNKHMNADTDYSIAEAAFNKG
ETAMTINGPWAWSNIDTSKVNYGVTVLPTFKGQPSKPFVGVLSAGINAASPNKEL
AKEFLENYLLTDEGLEAVNKDKPLGAVALKSYEEELAKDPRIAATMENAQKGEIM
PNIPQMSAFWYAVRTAVINAASGRQTVDEALKDAQTNSSSNNNNNNNNNNLGIE
GRISEFPELNTARGPIDTADLGVTLMHEHVFIMTTEIAQNYPEAWGDEDKRVAGAI
ARLGELKARGVDTIVDLTVIGLGRYIPRIARVAAATELNIVVATGLYTYNDVPFYF
HYLGPGAQLDGPEIMTDMFVRDIEHGIADTGIKAGILKCATDEPGLTPGVERVLRA
VAQAYKRTGAPISTHTHAGLRRGLDQQRIFAEEGVDLSRVVIGHCGDSTDVGYLE
ELIAAGSYLGMDRFGVDVISPFQDRVNIVARMCERGHADKMVLSHDACCYFDAL
PEELVPVAMPNWHYLHIHNDVIPALKQHGVTDEQLHTMLVDNPRRIFERQGGYQ
<SEQ ID NO: 7; PRT; Mycobacterium tuberculosis>
MRSRPTKSRKSEYQIRSWSSCLSRAFPLDSKKSTVAAMISPERRSGYLRVKWLGLS
SIRIGSRAVRSTTRVTLLHCGPARHRLRPSISDVPELNTARGPIDTADLGVTLMHEH
VFIMTTEIAQNYPEAWGDEDKRVAGAIARLGELKARGVDTIVDLTVIGLGRYIPRI
ARVAAATELNIVVATGLYTYNDVPFYFHYLGPGAQLDGPEIMTDMFVRDIEHGIA
DTGIKAGILKCATDEPGLTPGVERVLRAVAQAHKRTGAPISTHTHAGLRRGLDQQ
RIFAEEGVDLSRVVIGHCGDSTDVGYLEELIAAGSYLGMDRFGVDVISPFQDRVNI
VARMCERGHADKMVLSHDACCYFDALPEELVPVAMPNWHYLHIHNDVIPALKQ
HGVTDEQLHTMLVDNPRRIFERQGGYQ
<SEQ ID NO: 8; PRT; Mycobacterium tuberculosis>
MRSRPTKSRKSEYQIRSWSSCLSRAFPLDSKKSTVAAMISPERRSGYLRVKWLGLS
SIRIGSRAVRSTTRVTLLHCGPARHRLRPSISDVPELNTARGPIDTADLGVTLMHEH
VFIMTTEIAQNYPEAWGDEDKRVAGAIARLGELKARGVDTIVDLTVIGLGRYIPRI
ARVAAATELNIVVATGLYTYNDVPFYFHYLGPGAQLDGPEIMTDMFVRDIEHGIA
DTGIKAGILKCATDEPGLTPGVERVLRAVAQAHKRTGAPISTHTHAGLRRGLDQQ
RIFAEEGVELSRVVIGHCGDSTDVGYLEELIAAGSYLGMDRFGVDVISPFQDRVNI
VARMCERGHADKMVLSHDACCYFDALPEELVPVAMPNWHYLHIHNDVIPALKQ
HGVTDEQLHTMLVDNPRRIFERQGGYQ
<SEQ ID NO: 9; PRT; Mycobacterium tuberculosis>
MTLLHCGPARHRLRPSISDVPELNTARGPIDTADLGVTLMHEHVFIMTTEIAQNYP
EAWGDEDKRVAGAIARLGELKARGVDTIVDLTVIGLGRYIPRIARVAAATELNIVV
ATGLYTYNDVPFYFHYLGPGAQLDGPEIMTDMFVRDIEHGIADTGIKAGILKCATD
EPGLTPGVERVLRAVAQAHKRTGAPISTHTHAGLRRGLDQQRIFAEEGVDLSRVVI
GHCGDSTDVGYLEELIAAGSYLGMDRFGVDVISPFQDRVNIVARMCERGHADKM
VLSHDACCYFDALPEELVPVAMPNWHYLHIHNDVIPALKQHGVTDEQLHTMLVD
NPRRIFERQGGYQ
<SEQ ID NO: 10; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 11; PRT; Mycobacterium tuberculosis>
MPQLNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 12; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIMARMCERGHADKMVLSHDACCYFDALPEELV
PVAMPNWHYLHIHNDVIPALKQHGVTDEQLHTMLVDNPRRIFERQGGYQ
<SEQ ID NO: 13; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PAAMPNWHYLHIHNDVIPALKQHGVTDEQLHTMLVDNPRRIFERQGGYQ
<SEQ ID NO: 14; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVELSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 15; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLFTYNDVPFYFHYLGP
GAQLDGPEIMTDMFVRDIEHGIADTGIKAGILKCATDEPGLTPGVERVLRAVAQAH
KRTGAPISTHTHAGLRRGLDQQRIFAEEGVDLSRVVIGHCGDSTDVGYLEELIAAG
SYLGMDRFGVDVISPFQDRVNIVARMCERGHADKMVLSHDACCYFDALPEELVP
VAMPNWHYLHIHNDVIPALKQHGVTDEQLHTMLVDNPRRIFERQGGYQ
<SEQ ID NO: 16; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDAGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 17; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDLPFYFHYLGP
GAQLDGPEIMTDMFVRDIEHGIADTGIKAGILKCATDEPGLTPGVERVLRAVAQAH
KRTGAPISTHTHAGLRRGLDQQRIFAEEGVDLSRVVIGHCGDSTDVGYLEELIAAG
SYLGMDRFGVDVISPFQDRVNIVARMCERGHADKMVLSHDACCYFDALPEELVP
VAMPNWHYLHIHNDVIPALKQHGVTDEQLHTMLVDNPRRIFERQGGYQ
<SEQ ID NO: 18; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQAGYQ
<SEQ ID NO: 19; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDQFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 20; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRANIVARMCERGHADKMVLSHDACCYFDALPEELV
PVAMPNWHYLHIHNDVIPALKQHGVTDEQLHTMLVDNPRRIFERQGGYQ
<SEQ ID NO: 21; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTNEQLHTMLVDNPRRIFERQGGYQ
<SEQ ID NO: 22; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDTTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 23; PRT; Mycobacterium tuberculosis>
MPELNTARGPIDTADLGVTLMHEHVFIMATEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 24; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMADMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 25; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGFYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 26; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGVPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 27; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLQIHNDVIPALKQHGVTDEQLHTMLVDNPRRIFERQGGYQ
<SEQ ID NO: 28; PRT; Mycobacterium tuberculosis>
MPELNTARGPIDTADLGVTLMHEHVFIMTTEIAQNYPEAWGDEDKRVAGAIARLG
ELKARGVDTIVDLKVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 29; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPLQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 30; PRT; Mycobacterium tuberculosis>
MPELNTARGPIDTADLGVTLMHEHVLIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 31; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPLYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 32; PRT; Mycobacterium tuberculosis>
MPELNTARGPIDTADLGVTLMHEHVFIMTTEIAQKYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 33; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTHNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 34; PRT; Mycobacterium tuberculosis>
MPELNTARGPIDTADLGVMLMHEHVFIMTTEIAQNYPEAWGDEDKRVAGAIARL
GELKARGVDTIVDLTVIGLGRYIPRIARVAAATELNIVVATGLYTYNDVPFYFHYL
GPGAQLDGPEIMTDMFVRDIEHGIADTGIKAGILKCATDEPGLTPGVERVLRAVAQ
AHKRTGAPISTHTHAGLRRGLDQQRIFAEEGVDLSRVVIGHCGDSTDVGYLEELIA
AGSYLGMDRFGVDVISPFQDRVNIVARMCERGHADKMVLSHDACCYFDALPEEL
VPVAMPNWHYLHIHNDVIPALKQHGVTDEQLHTMLVDNPRRIFERQGGYQ
<SEQ ID NO: 35; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVGNPRRIFERQGGYQ
<SEQ ID NO: 36; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQHIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 37; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIVERQGGYQ
<SEQ ID NO: 38; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGRCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 39; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGRTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 40; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSSLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 41; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILXCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 42; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDERGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 43; PRT; Mycobacterium tuberculosis>
MPELNTARRPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 44; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRCIFERQGGYQ
<SEQ ID NO: 45; PRT; Mycobacterium tuberculosis>
MPELNTARGPIDTADLGVTLMHEHVFIMTTEIAQNYPEAWGDEDKRVAGAIARLG
ELKVRGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGVPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 46; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYKKVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 47; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCCFDALPEELV
PVAMPNWHYLHIHNDVIPALKQHGVTDEQLHTMLVDNPRRIFERQGGYQ
<SEQ ID NO: 48; PRT; Mycobacterium tuberculosis>
MPELNTARGPIDTADLGVTLMHEHVFIMTTEIAQNYPEAWGDEDKRVAGAIARLG
ELKVRGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGVPISTHTHAGLRRGLDQQRIFAEEGVDLSRVIIGHCGDSTDVGYLEELIAAG
SYLGMDRFGVDVISPFQDRVNIVARMCERGHADKMVLSHDACCYFDALPEELVP
VAMPNWHYLHIHNDVIPALKQHGVTDEQLHTMLVDNPRRIFERQGGYQ
<SEQ ID NO: 49; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLCTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVELSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 50; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVELSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFIFERQGGYQ
<SEQ ID NO: 51; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYCDALPGELV
PVAMPNWHYLHIHNDVIPALKQHGVTDEQLHTMLVDNPRRIFERQGGYQ
<SEQ ID NO: 52; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTHKHVPFYFHYLG
PGAQLDGPEIMTDMFVRDIEHGIADTGIKAGILKCATDEPGLTPGVERVLRAVAQA
HKRTGAPISTHTHAGLRRGLDQQRIFAEEGVDLSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFERQGGYQ
<SEQ ID NO: 53; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGRYIPRIARVAAATELNIVVATGLYTYNDVPFYFHYLG
PGAQLDGPEIMTDMFVRDIEHGIADTGIKAGILKCATDEPGLTPGVERVLRAVAQA
HKRTGAPISTHTHAGLRRGLDQQRIFAKEGVELSRVVIGHCGDSTDVGYLEELIAA
GSYLGMDRFGVDVISPFQDRVNIVARMCERGHADKMVLSHDACCYFDALPEELV
PVAMPNWHYLHIHNDVIPALKQHGVTDEQLHTMLVDNPRRIFIFERQGGYQ
<SEQ ID NO: 54; PRT; Mycobacterium canettii>
MPELNTARGPIDTADLGVTLMHEHVFIMTTEIAQNYPEAWGDEDKRVAGAIARLG
ELKVRGVDTIVDLTVIGLGRYIPRIARVAAATELNIVVATGLYTYNDVPFSFHYLGP
GARLGGPEIMTDMFVRDIEHGIADTGIKAGILKCATDEPGLTPGVERVLRAVAQAH
KRTGVPISTHTHAGLRRGLDQQRIFAEEGVDLSRVIIGHCGDSTDVGYLEELIAAGS
YLGMDRFGVDVISPFQDRVNIVARMCERGHADKMVLSHDACCYFDALPEELVPV
AMPNWHYLHIHNDVIPALKQRGVTDEQLHTMLVDNPRRIFERHGGYQ
<SEQ ID NO: 55; PRT; Mycobacterium tuberculosis>
MPELNTARGPIDTADLGVTLMHEHVFIMTTEIAQNYPEAWGDEDKRVAGAIARLG
ELKARGVDTIVDLTVIGLGXYIPRIAXXXAATELNIVVATXLYTYNDVPFYFHYLG
PGAQLDGPEIMTDMFVRDIEHGIADTGIKAGILKCATDEPGLTPGVERVLRAVAQA
HKRTGAPISTHTHAGXXXGLDQQRIFAEXGVDLSRVVIGHCGDSXDVGYLEELIA
AGSYLGMDRFGVDVISPFQDRVNIVARMCERGHADKMVLSHDACCYFDALPEEL
VPVAMPNWHYLHIHNDVIPALKQHGVTDEQLHTXLVDNPRRIFERQGGYQ
<SEQ ID NO: 56; PRT; Mycobacterium tuberculosis>
MHEHVFIMTTEIAQNYPEAWGDEDKRVAGAIARLGELKARGVDTIVDLTVIGLGR
YIPRIARVAAATELNIVVATGLYTYNDVPFYFHYLGPGAQLDGPEIMTDMFVRDIE
HGIADTGIKAGILKCATDEPGLTPGVERVLRAVAQAHKRTGAPISTHTHAGLRRGL
DQQRIFAEEGVDLSRVVIGHCGDSTDVGYLEELIAAGSYLGMDRFGVDVISPFQDR
VNIVARMCERGHADKMVLSHDACCYFDALPEELVPVAMPNWHYLHIHNDVIPAL
KQHGVTDEQLHTMLVDNPRRIFERQGGYQ
<SEQ ID NO: 57; PRT; Mycobacterium tuberculosis>
MVGDRDRVLEHVFIMTTEIAQNYPEAWGDEDKRVAGAIARLGELKARGVDTIVD
LTVIGLGRYIPRIARVAAATELNIVVATGLYTYNDVPFYFHYLGPGAQLDGPEIMT
DMFVRDIEHGIADTGIKAGILKCATDEPGLTPGVERVLRAVAQAHKRTGAPISTHT
HAGLRRGLDQQRIFAEEGVDLSRVVIGHCGDSTDVGYLEELIAAGSYLGMDRFGV
DVISPFQDRVNIVARMCERGHADKMVLSHDACCYFDALPEELVPVAMPNWHYLH
IHNDVIPALKQHGVTDEQLHTMLVDNPRRIFERQGGYQ
<SEQ ID NO: 58; PRT; Mycobacterium lacus>
MPVLNTARGPIDTADLGVTLMHEHVFIMTTEIAQNYPEAWGDEERRVADAITRLG
ELKARGVDTIVDLTVIGLGRYIPRIARVASATELNIVVATGLYTYNDVPFYFHYLGP
GGMLDGPEIMTDMFVRDIEQGIADTGIKAGILKCATDEPGVTPGVERVLRAVAQA
HKRTGVPISTHTHAGLRRGLEQQRIFAEEGVDLSRVIIGHSGDSTDVGYLEELIANG
SYLGMDRFGIDVISPFEDRVNIVAQMCERGHADKMVLSHDANCYFDALPEELVAV
AAPNWHYLHIHNDVIPALKERGVTDEQLHTMLVDNPRRIFERQGGY
<SEQ ID NO: 59; PRT; Mycobacterium avium>
MSELNTARGPIDTAALGVTLMHEHVFIMTTEIAQNYPEAWGDEEQRVADAIDRLN
ELKARGVDTIVDLTVIGLGRYIPRIARVAAATELNIVVATGLYTYNDVPFYFHYLG
PGAELGGPEIMTDMFVRDIEQGIADTGIKAGILKCATDAPGVTPGVERVLRAVAQA
HKRTGVPISTHTHAGLRRGLDQQRIFEEEGVDLSRVVIGHSGDSTDVGYLEELIAA
GSYLGMDRFGIDAISPFEDRVNIVAQMCERGHADKMVLSHDANCYFDALPEELVP
QVMPNWHYLHIHNDVIPALKQRGVTDEQLHTMLVDNPRRIFERQGPYG
<SEQ ID NO: 60; PRT; Mycobacterium tuberculosis>
MTTEIAQNYPEAWGDEDKRVAGAIARLGELKARGVDTIVDLTVIGLGRYIPRIARV
AAATELNIVVATGLYTYNDVPFYFHYLGPGAQLDGPEIMTDMFVRDIEHGIADTGI
KAGILKCATDEPGLTPGVERVLRAVAQAHKRTGAPISTHTHAGLRRGLDQQRIFAE
EGVDLSRVVIGHCGDSTDVGYLEELIAAGSYLGMDRFGVDVISPFQDRVNIVARM
CERGHADKMVLSHDACCYFDALPEELVPVAMPNWHYLHIHNDVIPALKQHGVTD
EQLHTMLVDNPRRIFERQGGYQ
<SEQ ID NO: 61; PRT; Mycobacterium haemophilum>
MSELNTARGPISTADLGVTLMHEHVFIMTTEIAQNYPEAWGDEEQRVADAIARLS
ELKARGVDTIVDLTVIGLGRYIPRIARVAAATELNIVVATGLYTYNDVPFYFHYLG
PGAPLNGPEIMTDMFVRDIEQGVADTGIKAGILKCATDEPGITPGVERVLRAVAQA
HKRTGVPISTHTHAGLRRGLEQQRIFAEEGVDLSRVIIGHSGDSTDVGYLEELIANG
SYLGMDRFGVDVILPFEDRVSIVAQMCERGHADKMVLSHDANCYFDALPEALQP
VAAPNWHYLHIHNDVIPALKERGVTDEQLHTMLVDNPRRIFERQGTYE
<SEQ ID NO: 62; PRT; Mycobacterium sp.>
MSELNTARGPIDTADLGVTLMHEHVFIMTTEVVQNYPEAWGDEDKRVADAVARL
NELKSRGVDTIVDLTVIGLGRYIPRIARVAAQTELNIVVATGLYTYNDVPFCFHYL
GPGTQLDGPETMTDLFVRDIEQGIADTGIKAGILKCATDEPGITPGVERVLRAVAQ
AHKRTGVPISTHTHAGLRRGLEQQRIFEEEGVDLSRVVIGHSGDSTDVGYLEELVA
AGSYLGMDRFGLDVILPFEDRVNIVAQMCERGHADKMVLSHDASCYFDALPEAL
LPVAAPNWNYLHIHNDVIPALKQRGVTDEQLHTMLVDNPRRIFERQGGYE
<SEQ ID NO: 63; PRT; Mycobacterium shimoidei>
MSQLNTARGPIDTADLGVTLMHEHVFIMTTEVAQNYPEAWGDEEQRVADAITRL
NELKARGVDTIVDLTVIGLGRYIPRIARVAAATELNIVVATGLYTYNDVPFRFHYQ
GPGTQLDGPEIMTDMFVRDIEQGIADTGIKAAILKCATDEPGITPGVERVLRAVAQ
AHKRTGVPISTHTHAGLRRGLEQQRIFEEEGADLSRVIIGHSGDSTDVGYLEELIAA
GSYLGMDRFGIDVISPFEDRVNIVARMCERGHADKMVLSHDANCYFDALPEELLP
VVAPNWHYLHIHNDVIPALKERGVTDEQLHTMLVDNPRRIFERTGAYE
<SEQ ID NO: 64; PRT; Mycobacterium shimoidei>
MSQLNTARGPIDTADLGVTLMHEHVFIMTTEVAQNYPEAWGDEEQRVADAITRL
NELKARGVDTIVDLTVIGLGRYIPRIARVAAATELNIVVATGLYTYNDVPFRFHYQ
GPGTQLDGPEIMTDMFVRDIEQGIADTGIKAAILKCATDEPGITPGVERVLRAVAQ
AHKRTGVPISTHTHAGLRRGLEQQRIFEEEGVDLSRVIIGHSGDSTDVGYLEELIAA
GSYLGMDRFGIDVISPFEDRVNIVAQMCERGHADKMVLSHDANCYFDALPEELLP
VVAPNWHYLHIHNDVIPALKERGVTDEQLHTMLVDNPRRIFERTGAYE
<SEQ ID NO: 65; PRT; Mycobacterium bohemicum>
MSELNTARGPIDTADLGVTLMHEHVFIMTTEIAQNYPEAWGDEDQRVADAITRLN
ELKARGVDTIVDLTVIGLGRYIPRIARVAAATELNIVVATGLYTYNDVPFCFHYLG
PGAALGGPEVMTDMFVRDIEQGIADTGVKAGILKCATDEPGITPGVERVLRAVAQ
AHRRTGVPISTHTHAGLRRGLEQQRIFEEEGVDLSRVVIGHSGDSTDVGYLEELIAA
GSYLGMDRFGIDVISPFEDRVNIVAQMCERGHADKMVLSHDANCYFDALPEELLA
VAAPNWHYLHIHNDVIPALKERGVTDEQLHTMLVDNPRRIFERRGAY
<SEQ ID NO: 66; PRT; Mycobacterium kansasii>
MSELNTARGPIDTADLGVTLMHEHVFIMTTEVVQNYPEAWGDEDKRVADAVARL
NELKSRGVDTIVDLTVIGLGRYIPRIARVAAQTELNIVVATGLYTYNDVPFCFHYL
GPGTQLDGPETMTDLFVRDIEQGIADTGIKAGILKCATDDPGITPGVERVLRAVAQ
AHKRTGVPISTHTHAGLRRGLEQQRIFEEEGVDLSRVVIGHSGDSTDVGYLEELVA
AGSYLGMDRFGLDVILPFEDRVNIVAQMCERGHADKMVLSHDASCYFDALPEAL
LPVAAPNWNYLHIHNDVIPALKQRGVTDEQLHTMLVDNPRRIFERQGGYE
<SEQ ID NO: 67; PRT; Mycobacterium avium>
MSELNTARGPIDTAALGVTLMHEHVFIMTTEIAQNYPEAWGDEEQRVADAIDRLN
ELKARGVDTIVDLTVIGLGRYIPRIARVAAATELNIVVATGLYTYNDVPFCFHYLG
PGAELGGPEIMTDMFVRDIEQGIADTGIKAGILKCATDAPGVTPGVERVLRAVAQA
HKRTGVPISTHTHAGLRRGLDQQRIFEEEGVDLSRVVIGHSGDSTDVGYLEELIAA
GSYLGMDRFGIDAISPFEDRVNIVAQMCERGHADKMVLSHDANCYFDALPEELVP
QVMPNWHYLHIHNDVIPALKQRGVTDEQLHTMLVDNPRRIFERQGPYG
<SEQ ID NO: 68; PRT; Mycobacterium sp.>
MGYVSQPGIASELNTARGPIDTADLGVTLMHEHVFIMTTEIALNYPEAWGDEEKR
VADAVTRLNELKARGVDTIVDLTVIGLGRYIPRIAKVAAATELNIVVATGLYTYND
VPFCFHYMGPGAQLGGPEIMTDMFVRDIEQGIADTGIKAGILKCATDEPGITPGVE
RVLRAVAQAHKRTGVPISTHTHAGLRRGLEQQRIFEEEGVDLSRVIIGHSGDSTDV
GYLEELIAAGSYLGMDRFGIDVILPFEERVNIVATMCERGHADKMVLSHDANCYF
DALPEEMLAVAAPNWHYLHIHNDVIPALKQRGVTDEQLHTMLVDNPRRIFERQG
AYQ
<SEQ ID NO: 69; PRT; Mycobacterium sp.>
MSELNTARGPIDTADLGVTLMHEHVFIMTTEIAQNYPEAWGDEEKRVADAITRLN
ELKSRGVHTIVDLTVIGLGRYIPRIARVAAATELNIVVATGLYTYNDVPFRFHYQGP
GCMLEGPEIMTDMFVRDIEQGIADTGVKAGILKCATDEPGLTPGVERVLRAVAQA
HKRTGVPISTHTHAGLRRGLEQQRVFAEEGVDLSRVIIGHSGDSTDVGYLEELIAA
GSYLGMDRFGIDLILPFEDRVNIVATMCERGHADKMVLSHDANCYFDALPEELVP
VATPNWHYLHIHNDVIPALKERGVTDEQVHTMLVDNPRRIFERQGGY
<SEQ ID NO: 70; PRT; Mycobacterium haemophilum>
MSELNTARGPISTADLGVTLMHEHVFIMTTEIAQNYPEAWGDEEQRVADAIARLS
ELKARGVDTIVDLTVIGLGRYIPRIARVAAATELNIVVATGLYTYNDVPFYFHYLG
PGAPLNGPEIMTDMFVRDIEQGVADTGIKAGILKCATDEPGITPGVERVLRAVAQA
HKRTGVPISTHTHAGLRRGLEQQRIFAEEGVDLSRVIIGHSGDSTDVGYLEELIANG
SYLGMDRFGVDVILPFEDRVSIVAQMCERGHAGKMVLSHDANCYFDALPEALQP
VAAPNWHYLHIHNDVIPALKERGVTDEQLHTMLVDNPRRIFERQGTYE
<SEQ ID NO: 71; PRT; Mycobacterium persicum>
MSELNTARGPIDTADLGVTLMHEHVFIMTTEVAQNYPEAWGDEDRRVADAVARL
NELKSRGVDTIVDLTVIGLGRYVPRIARVAAQTELNIVVATGLYTYNDVPFCFHYL
GPGTQLDGPETMTDLFVRDIEQGIADTGIKAGILKCATDEPGITPGVERVLRAVAQ
AHKRTGVPISTHTHAGLRRGLEQQRIFEEEGVDLSRVVIGHSGDSTDVGYLEELIA
AGSYLGMDRFGLDVILPFEERVAIVAQMCERGHADKMVLSHDATCYFDALPEAL
LPVAAPNWHYLHIHNDVIPALKQRGVTDEQLHTMLVDNPRRIFERQGGYD
<SEQ ID NO: 72; PRT; Mycobacterium persicum>
MSELNTARGPIDTADLGVTLMHEHVFIMTTEVAQNYPEAWGDEDRRVADAVARL
NELKSRGVDTIVDLTVIGLGRYVPRIARVAAETELNIVVATGLYTYNDVPFCFHYL
GPGTQLDGPETMTDLFVRDIEQGIADTGIKAGILKCATDEPGITPGVERVLRAVAQ
AHKRTGVPISTHTHAGLRRGLEQQRIFEEEGVDLSRVVIGHSGDSTDVGYLEELIA
AGSYLGMDRFGLDVILPFEERVAIVAQMCERGHADKMVLSHDATCYFDALPEAL
LPVAAPNWHYLHIHNDVIPALKQRGVTDEQLHTMLVDNPRRIFERQGGYD
<SEQ ID NO: 73; PRT; Mycobacterium sp.>
MSELNTARGPIDTADLGVTLMHEHVFIMTTEVVQNYPDAWGDEDKRVADAVAR
LNELKSRGVDTIVDLTVIGLGRYIPRIARVAAQTELNIVVATGLYTYNDVPFCFHYL
GPGTQLDGPETMTDLFVRDIEQGIADTGIKAGILKCATDEPGITPGVERVLRAVAQ
AHKRTGVPISTHTHAGLRRGLEQQRIFEEEGVDLSRVVIGHSGDSTDVGYLEELIA
AGSYLGMDRFGLDVILPFEDRVNIVAQMCERGHADKMVLSHDANCYFDALPEAL
LPVAAPNWNYLHIHNDVIPALKQRGVTDEQLHTMLVDNPRRIFERQGAYE
<SEQ ID NO: 74; PRT; Mycobacterium sp.>
MSELNTARGPIDTADLGVTLMHEHVFIMTTEIALNYPEAWGDEEKRVADAVARL
NELKARGVDTIVDLTVIGLGRYIPRIAKVAASTDLNIVVATGLYTYNDVPFCFHYM
GPGAQLGGPEIMTDMFVRDIEQGIADTGIKAGILKCATDEPGITPGVERVLRAVAQ
AHKRTGVPISTHTHAGLRRGLEQQRIFEEEGVDLSRVIIGHSGDSTDVGYLEELIAA
GSYLGMDRFGIDVILPFEERVNIVATMCERGHADKMVLSHDANCYFDALPEEMLA
VAAPNWHYLHIHNDVIPALKQRGVTDEQLHTMLVDNPRRIFERQGAYQ
<SEQ ID NO: 75; PRT; Mycobacterium avium>
MSELNTARGPIDTAALGVTLMHEHVFIMTTEIAQNYPEAWGDEEQRVADAIDRLN
ELKARGVDTIVDLTVIGRGRYIPRIARVAAATELNIVVATGLYTYNDVPFCFHYLG
PGAELGGPEIMTDMFVRDIEQGIADTGIKAGILKCATDAPGVTPGVERVLRAVAQA
HKRTGVPISTHTHAGLRRGLDQQRIFEEEGVDLSRVVIGHSGDSTDVGYLEELIAA
GSYLGMDRFGIDAISPFEDRVNIVAQMCERGHADKMVLSHDANCYFDALPEELVP
QVMPNWHYLHIHNDVIPALKQRGVTDEQLHTMLVDNPRRIFERQGPYG
<SEQ ID NO: 76; PRT; Mycobacterium interjectum>
MSELNTARGAIDTADLGVTLMHEHVFIMTTEIALNYPEAWGDEEQRVADAIDRLN
ELKARGVDTIVDLTVIGLGRYIPRIARVAAATELNIVVATGLYTYNDVPFRFHYQG
PGAPLDGPEIMTDMFVRDIEEGIADTGVKAGILKCATDEPGVTPGVERVLRAVAQ
AHKRTGVPISTHTHAGLRRGLEQQRIFSEEGVDLSRVVIGHSGDTTDVGYLEELIA
AGSYLGMDRFGIDLISPFEDRVNIVATMCERGHADKMVLSHDANCYFDALPEELL
PVAAPNWHYLHIHNDVIPALKERGVTDEQLHTMLVDNPRRIFERQGAYE
<SEQ ID NO: 77; PRT; Mycobacterium sp.>
MSELNTARGLIDTADLGVTLMHEHVFIMTTEVVQNYPEAWGDEDQRVADAVARL
NELKARGVDTIVDLTVIGLGRYIPRIARVAAQTELNIVVATGLYTYNDVPFCFHYL
GPGTQLDGPETMTDLFVRDIEQGIADTGIKAGILKCATDEPGITPGVERVLRAVAQ
AHKRTGVPISTHTHAGLRRGLEQQRIFEEEGVDLSRVVIGHSGDSTDVGYLEELIA
AGSYLGMDRFGLDVILPFEDRVNIVARMCERGHADKMVLSHDATCYFDALPEAL
LPVAAPNWNYLHIHNDVIPALKQRGVTDEQLHTMLVDNPQRIFERQGAYE
<SEQ ID NO: 78; PRT; Mycobacterium persicum>
MSELNTARGPIDTADLGVTLMHEHVFIMTTEVAQNYPEAWGDEDRRVADAVARL
NELKSRGGDTIVDLTVIGLGRYVPRIARVAAQTELNIVVATGLYTYNDVPFCFHYL
GPGTQLDGPETMTDLFVRDIEQGIADTGIKAGILKCATDEPGITPGVERVLRAVAQ
AHKRTGVPISTHTHAGLRRGLEQQRIFEEEGVDLSRVVIGHSGDSTDVGYLEELIA
AGSYLGMDRFGLDVILPFEERVAIVAQMCERGHADKMVLSHDATCYFDALPEAL
LPVAAPNWHYLHIHNDVIPALKQRGVTDEQLHTMLVDNPRRIFERQGGYD
<SEQ ID NO: 79; PRT; Mycobacterium kansasii
MSELNPARGPIDTADLGVTLMHEHVFIMTTEVVQNYPDAWGDEDKRVADAVARL
NELKSRGVDTIVDLTVIGLGRYIPRIARVAAQTELNIVVATGLYTYNDVPFCFHYL
GPGTQLDGPETMTDLFVRDIEQGIADTGIKAGILKCATDEPGITPGVERVLRAVAQ
AHKRTGVPISTHTHAGLRRGLEQQRIFEEEGVDLSRVVIGHSGDSTDVGYLEELIA
AGSYLGMDRFGLDVILPFEDRVNIVAQMCERGHADKMVLSHDANCYFDALPEAL
LPVAAPNWNYLHIHNDVIPALKQRGVTDEQLHTMLVDNPRRIFERQGAYE
<SEQ ID NO: 80; PRT; Mycobacterium fragae>
MSQLNTARGPIDTGDLGVTLMHEHVFIMTTEIALNYPEAWGDEDQRVSDAVNRL
NELKSRGVDTIVDLTVIGLGRYIPRIARVAAETDLNIVVATGLYTYNDVPFRFHYQ
GPGTQLDGPEIMTDMFVRDIEEGIADTGIKAGILKCATDEPGITPGVERVLRAVAQ
AHKRTGVPISTHTHAGLRRGLEQQRIFEEEGVDLSRVIIGHSGDSTDIGYLEELIAAG
SYLGMDRFGIDVILPFEDRVNIVAQMCERGHADKMVLSHDANCYFDALPEELMP
VAAPNWHYLHIHNDVIPALKERGVTDEQLHTMLVDNPRRIFERQGAYQ
<SEQ ID NO: 81; PRT; Mycobacterium kubicao>
MSELNTARGPIDTANLGVTLMHEHVFIMTTEIIQNYPEAWGDEEKRVADAVARLN
ELKSRGVDSIVDLTVIGLGRYIPRIARVAAQTELNIVVATGIYTYNDVPFHFHYSGP
GTILNGPEVMVDMFVRDIEQGIADTGVKAGILKCATDEPGLTPGVERVLRAVAQA
HKRTGVPISTHTHAGLRRGLDQQRIFEEEGVDLSRVVIGHSGDSTDVGYLEELIASG
SYLGMDRFGLDLISPFEDRVNIVAQMCERGHADKMVLSHDANCYFDALPEEVVP
QVLPNWHYLHIHNDVIPALKERGVTDEQLHTMLVDNPRRIFERQGGYE
<SEQ ID NO: 82; PRT; Mycobacterium kubicae>
MSELNTARGPIDTANLGVTLMHEHVFIMTTEIIQNYPEAWGDEEKRVADAVARLN
ELKSRGVDSIVDLTVIGLGRYIPRIARVAAQTELNIVVATGIYTYNDVPFHFHYSGP
GTILNGPEVMVDMFVRDIEQGIADTGVKAGILKCATDEPGLTPGVERVLRAVAQA
HKRTGVPISTHTHAGLRRGLDQQRIFEEEGVDLSRVVIGHSGDSTDIGYLEELIASG
SYLGMDRFGLDLISPFEDRVNIVAQMCERGHADKMVLSHDANCYFDALPEEVVP
QVLPNWHYLHIHNDVIPALKERGVTDEQLHTMLVDNPRRIFERQGGYE
<SEQ ID NO: 83; PRT; Mycobacterium sp. E3198>
MSELNTARGAIDTADLGVTLMHEHVFIMTTEIALNYPEAWGDEEQRVADAIDRLN
ELKARGVDTIVDLTVIGLGRYIPRIARVAAATELNIVVATGLYTYNDVPFRFHYQG
PGAPLDGPEIMTDMFVRDIEEGIADTGVKAGILKCATDEPGVTPGVERVLRAVAQ
AHKRTGVPISTHTHAGLRRGLEQQRIFESEGVDLSRVVIGHSGDSTDVGYLEELIAA
GSYLGMDRFGIDLISPFEDRVNIVATMCERGHADKMVLSHDANCYFDALPEELLP
VAAPNWHYLHIHNDVIPALKERGVTDEQLHTMLVDNPRRIFERQGAYA
<SEQ ID NO: 84; PRT; Mycobacterium decipiens>
MSALNTARGPIDTADLGVTLMHEHVFIMTTEIAQNYPEGWGDEEQRVADAITRLS
ELKARGVDTIVDLTVIGLGRYIPRIARVAAATELNIVVATGLYTYNDVPFFFHYLGP
GGMLDGPEIMTDMFVRDIEEGIADTGIKAGILKCATDEPGITPGVERVLRAVAQAH
MRTGVPISTHTHAGLRRGLEQQRIFAEEGVDLSRVIIGHSGDSTDVGYLEELISNGS
YLGMDRFGIDLILPFEERVNIVARLCERGHADKMVLSHDANCYFDALPEALVPAA
APNWHYLHIHNDVIPALKERGVTDEQVRTMLVDNPRRIFERQGGYQ
<SEQ ID NO: 85; PRT; Mycobacterium sp.>
MSELNTARGSIDTADLGVTLMHEHVFIMTTEIALNYPEAWGDEEQRVADAITRLN
ELKARGVDTIVDLTVIGLGRYIPRIARVAAATELNIVVATGLYTYNDVPFCFHYMG
PGAQLGGPEIMTEMFVRDIEQGIADTGIKAGILKCATDEPGITPGVERVLRAVAQA
HKQTGVPISTHTHAGLRRGLEQQRIFEEEGVDLSRVIIGHSGDSTDVGYLEELISAG
SYLGMDRFGIDVILPFEDRVNIVATMCERGHADKMVLSHDANCYFDALPEELLPV
AAPNWHYLHIHNDVIPALKERGVTDEQLHTMLVDNPRRIFERQGSY
<SEQ ID NO: 86; PRT; Mycobacterium sp. E342
MSELNTARGPIDTADLGVTLMHEHVFIMTTEIALNYPEAWGDEDKRVADAIARLN
ELKSRGVDTIVDLTVIGLGRYIPRIARVAAATDLNIVVATGLYTYNDIPFRFHYEGP
GGMLDGPEIMTDMFVKDIEQGIADTGVKAGILKCATDEPGITPGVERVLRAVAQA
HKRTGVPISTHTHAGLRRGLEQQRIFEEEGVDLTRVVIGHSGDSTDIGYLEELIAAG
SYLGMDRFGLDVISPFEERVKIVAQMCERGHADKMVLSHDANCYFDALPEELVPQ
MAPNWHYLHIHNDVIPALKQRGVTDEQLHTMLVDNPRRIFERQGAY
<SEQ ID NO: 87; PRT; Mycobacterium sp. 852002-10029_SCH5224772>
MSELNTARGSIDTAQLGVTLMHEHVFIMTTEIAENYPEAWGDEERRVADAIDRLN
ELKSRGVDTIVDLTVIGLGRYIPRIARVAAATELNIVVATGLYTYNDVPFYFHYLGP
GAELGGPEIMTDMFVRDIEQGIADTGVKAGILKCATDAPGVTPGVERVLRAVAQA
HKRTGVPISTHTHAGLRRGLEQQRIFEEEGVDLSRVVIGHSGDSTDVGYLEELIAA
GSYLGMDRFGIDAISPFEDRVNIVAQMCERGHADKMVLSHDANCYFDALPEELVP
QVMPNWHYLHIHNDVIPALKERGVTDEQLHTMLVDNPRRIFEQTGAY
<SEQ ID NO: 88; PRT; Mycobacterium gastri>
MSELNTARGPIDTADLGVTLMHEHVFIMTTEVVQNYPDAWGDEDKRVADAVGR
LNVLKSRGVDTIVDLTVIGLGRYIPRIARVAAQTELNIVVATGLYTYNDVPFCFHY
LGPGTQLDGPEIMTDLFVRDIEQGIADTGIKAGILKCATDEPGITPGVERVLRAVAQ
AHKRTGVPISTHTHAGLRRGLEQQRIFEEEGVDLSRVIIGHSGDSTDVGYLEELVA
AGSYLGMDRFGIDVILPFEDRVNIVATMCERGHADKMVLSHDANCYFDALPEALL
PVAAPNWHYLHIHNDVIPALTQRGVTDEQLHTMLVDNPRRIFERQGAYE
<SEQ ID NO: 89; PRT; Mycobacterium riyadhenses>
MSELNTARGTIDTADLGVTLMHEHVFIMTTEIAQNYPEGWGDEEKRVADAITRLN
ELKARGVDTIVDLTVIGLGRYIPRIAQVAAQTELNIVVATGLYTYNDVPFCFHFMG
PGGMLDGPEIMTDMFVRDIEQGIADTGIKAAILKCATDEPGVTPGVERVLRAVAQ
AHKRTGVPISTHTHAGLRRGLEQQRIFEEEGVDLSRVIIGHSGDSTDVGYLEELIAA
GSYLGMDRFGVDVISPFEDRVNIVAQMCERGHADKMVLSHDANCYFDALPEALV
PVVTPNWHYLHIHNDVIPALKQRGVTDEQVQTMLVDNPRRIFERQGGY
<SEQ ID NO: 90; PRT; Mycobacterium nebraskenso>
MSELNTARGPIDTADLGVTLMHEHVFIMTTEIALNYPEAWGDEEKRVADAIARLN
ELKARGVDTIVDLTVIGLGRYIPRIARVAAATELNIVVATGLYTYNDVPFRFHYEG
PGGMLDGPEIMTEMFVRDIEQGIADTGVKAGILKCATDEPGITPGVERVLRAVAQA
HKRTGVPISTHTHAGLRRGLEQQRIFEEEGVDLTRVVIGHSGDSTDVGYLEELVAA
GSYLGMDRFGLDVISPFEERVGIVATMCERGHADKMVLSHDANCYFDALPEELVP
QMAPNWHYLHIHNDVIPALKERGVTDEQLHTMLVDNPRRIFERQGAYE
<SEQ ID NO: 91; PRT; Mycobacterium kansasii>
MSELNTARGPIDTADLGVTLMHEHVFIMTTEVVQNYPDAWGDEDKRVADAVAR
LNELKSRGVDTIVDLTVIGLGRYIPRIARVAAQTELNIVVATGLYTYNDVPFCFHYL
GPGTQLDGPETMTDLFVRDIEQGIADTGIKAGILKCATDEPGITPGVERVLRAVAQ
AHKHTGVPISTHTHAGLRRGLEQQRIFEEEGVDLSRVVIGHSGDSTHVGYLEELIA
AGSYLGMDRFGLDVILPFEDRVNIVAQMCERGHADKMVLSHDANCYFDALPEAL
LPVAAPNWNYLHIHNDVIPALKQRGVTDEQLHTMLVDNPRRIFERQGAYE
<SEQ ID NO: 92; PRT; Mycobacterium sp. 852002-40037_SCH5390672>
MSELNTARGPIDTADLGVTLMHEHVFIMTTEIAQNYPEAWGDEEKRVADAIARLN
ELKARGVDTIVDLTVIGLGRYIPRIARVAAATELNIVVATGLYTYNDIPFRFHYEGP
GGMLGGPEIMTDMFVKDIEVGIADTGVKAGILKCATDEPGITPGVERVLRAVAQA
HKRTGVPISTHTHAGLRRGLEQQRIFEEEGVDLTRVVIGHSGDSTDIGYLEELIAAG
SYLGMDRFGLDVISPFEERVRIVAEMCERGHADKMVLSHDANCYFDALPEELVPQ
MAPNWHYLHIHNDVIPALKERGVTDEQLHTMLVDNPRRIFERQGSYE
<SEQ ID NO: 93; PRT; Mycobacterium kubicao>
MSELNTARGPIDTANLGVTLMHEHVFIMTTEIIQNYPEAWGDEEKRVADAVARLN
ELKSRGVDSIVDLTVIGLGRYIPRIARVAAQTDLNIVVATGIYTYNDVPFHFHYNGP
GTILNGPEVMVDMFVRDIEQGIADTGVKAGILKCATDEPGLTPGVERVLRAVAQA
HKRTGVPISTHTHAGLRRGLDQQRIFEEEGVDLSRVVIGHSGDSTDIGYLEELIASG
SYLGMDRFGLDLISPFEDRVNIVAQMCERGHADKMVLSHDANCYFDALPEEVVP
QVLPNWHYLHIHNDVIPALKERGVTDEQLHTMLVDNPRRIFERQGGYE
<SEQ ID NO: 94; PRT; Mycobacterium marinum>
MSALNTARGPIDTADLGVTLMHEHVFIMTTEVAQNYPEAWGDEEKRVADAITRL
NELKSRGVDTIVDLTVIGLGRYIPRIARIAAATDLNIVVATGLYTYNDVPYRFHYQ
GPGGMLDGPEIMTDMFVRDIEGGIADTGVKAGILKCATDEPGITPGVERVLRAVA
QTHKRTGVPISTHTHAGLRRGLEQQRIFEEEGVDLSRVIIGHSGDSTDVGYLEELIA
AGSYLGMDRFGIDLILPFEDRVNIVATMCERGHADKMVLSHDANCYFDALPEELT
AVATPNWHYLHIHNDVIPALKERGVTDEQVHTMLVDNPRRIFERQGGYQ
<SEQ ID NO: 95; PRT; Mycobacterium malmoenses>
MSELNTARGPIDTADLGVTLMHEHVFIMTTEIALNYPEAWGDEDRRVADAIDRLN
ELKSRGVDTIVDLTVIGLGRYIPRIARVAAATELNIVVATGLYTYNDVPFRFHYEGP
GGMLDGPEIMTEMFVRDIEQGIADTGIKAGILKCATDEPGITPGVERVLRAVAQAH
KRTGVPISTHTHAGLRRGLEQQRIFEEEGVDLSRVVIGHSGDSTDVGYLEELIAAGS
YLGMDRFGLDVISPFEERVKIVATMCERGHADKMVLSHDANCYFDALPEDLVPQ
MAPNWHYLHIHNDVIPALKERGVTDEQLHTMLVDNPRRIFERQGAYE
<SEQ ID NO: 96; PRT; Mycobacterium scrofulaceum>
MSELNTARGPIDTADLGVTLMHEHVFIMTTEIALNYPEAWGDEDRRVADAIDRLN
ELKSRGVDTIVDLTVIGLGRYIPRIARVAAATELNIVVATGLYTYNDVPFRFHYEGP
GGLLDGPEIMTEMFVRDIEQGIADTGIKAGILKCATDEPGITPGVERVLRAVAQAH
KRTGVPISTHTHAGLRRGLEQQRIFEEEGVDLSRVVIGHSGDSTDVGYLEELIAAGS
YLGMDRFGLDVISPFEERVKIVATMCERGHADKMVLSHDANCYFDALPEDLVPQ
MAPNWHYLHIHNDVIPALKERGVTDEQLHTMLVDNPRRIFERQGAYE
<SEQ ID NO: 97; PRT; Mycobacterium marinum>
MSALNTARGPIDTADLGVTLMHEHVFIMTTEVAQNYPEAWGDEEKRVADAITRL
NELKSRGVDTIVDLTVIGLGRYIPRIARIAAATDLNIVVATGLYTYNDVPYRFHYQ
GPGGMLDGPEIMTDMFVRDIEQGIADTGVKAGILKCATDEPGITPGVERVLRAVA
QTHKRTGVPISTHTHAGLRRGLEQQRIFEEEGVDLSRVIIGHSGDSTDVGYLEELIA
AGSYLGMDRFGIDLILPFEDRVSIVATMCERGHADKMVLSHDANCYFDALPEELT
AVATPNWHYLHIHNDVIPALKERGVTDEQVHTMLVDNPRRIFERQGGYQ
<SEQ ID NO: 98; PRT; Mycobacterium sp. E2497>
MLELNTARGPIDTADLGVTLMHEHVFIMTTEIALNYPEAWGDEEKRVADAIARLN
ELKARGVDTIVDLTVIGLGRYIPRIARVAAATDLNIVVATGLYTYNDIPFRFHYEGP
GGMLGGPEIMTDMFVKDIEQGIADTGVKAGILKCATDEPGITPGVERVLRAVAQA
HKRTGVPISTHTHAGLRRGLEQQRIFEEEGVDLTRVVIGHSGDSTDIGYLEELIAAG
SYLGMDRFGLDVISPFEERVKIVAQMCERGHADKMVLSHDANCYFDALPEELVPQ
MAPNWHYLHIHNDVIPALKQRGVTDEQLHTMLVDNPRRIFERQGAY
<SEQ ID NO: 99; PRT; Mycobacterium simiae>
MSELNTARGAIDTADLGVTLMHEHVFIMTTEIAQNYPEAWGDEEKRVADAIARLN
ELKARGVDTIVDLTVIGLGRYIPRIARVAAATELNIVVATGIYTYNDVPFRFHYEGP
GGMLDGPEIMTEMFVRDIEQGIADTGVKAGILKCATDEPGITPGVERVLRAVAQA
HKRTGVPISTHTHAGLRRGLDQQKIFDEEGVDLTRVVIGHSGDSTDVGYLEELIAA
GSYLGMDRFGLDVISPFEERVKIVAQMCERGHADKMVLSHDANCYFDALPEELVP
QLAPNWHYLHIHNDVIPALKERGVTDEQLHTMLVDNPRRIFERTGAY
<SEQ ID NO: 100; PRT; Mycobacterium marinum>
MSALNTARGPIDTADLGVTLMHEHVFIMTTEVAQNYPEAWGDEEKRVADAITRL
NELKSRGVDTIVDLTVIGLGRYIPRIARIXAATDLNIVVATGLYTYNDVPYRFHYQ
GPGGMLDGPEIMTDMFVRDIEQGIADTGVKAGILKCATDEPGITPGVERVLRAVA
QTHKRTGVPISTHTHAGLRRGLEQQRIFEEEGVDLSRVIIGHSGDSTDVGYLEELIA
AGSYLGMDRFGIDLILPFEDRVNIVATMCERGHADKMVLSHDANCYFDALPEELT
AVATPNWHYLHIHNDVIPALKERGVTDEQVHTMLVDNPRRIFERQGGYQ
<SEQ ID NO: 101; PRT; Mycobacterium sp. E796>
MPELNTARGAIDTADLGVTLMHEHVFIMTTEIALNYPEAWGDEDQRVADAVNRL
NELKSRGVDTIVDLTVIGLGRYIPRIARVAAATELNIVVATGLYTYNDVPFRFHYL
GPGTQLDGPEIMTDMFVRDIEEGIADTGIKAGILKCATDEPGVTPGVERVLRAVAQ
AHKRTGVPISTHTHAGTRRGLEQQRIFEEEGVDLSRVVIGHSGDSTDLDYLEELIGN
GSYIGMDRFGIDVYLPFEDRVNTVARMCERGHADKMVLSHDANCYFDALPEELL
PVAAPNWHYLHIHNDVIPALKERGVTDEQLHTMLVDNPRRIFERQGAYS
<SEQ ID NO: 102; PRT; Pseudomonas sp. WBC-3>
MPLKNRLLARLSCVAAVVAATAAVAPLTLVSTAHAAAPQVRTSAPGYYRMLLGD
FEITALSDGTVALPVDKRLNQPAPKTQSALAKSFQKAPLETSVTGYLVNTGSKLVL
VDTGAAGLFGPTLGRLAANLKAAGYQPEQVDEIYITHMHPDHVGGLMVGEQLAF
PNAVVRADQKEADFWLSQTNLDKAPDDESKGFFKGAMASLNPYVKAGKFKPFSG
NTDLVPGIKALASHGHTPGHTTYVVESQGQKLALLGDLILVAAVQFDPSVTTQLD
SDSKSVAVERKKAFADAAKGGYLIAASHLSFPGIGHIRAEGKGYRFVPVNYSVVN
PK
<SEQ ID NO: 103; PRT; Pseudomonas diminuta>
GDRINTVRGPITISEAGFTLTHEHICGSSAGFLRAWPEFFGSRKALAEKAVRGLRRA
RAAGVRTIVDVSTFDIGRDVSLLAEVSRAADVHIVAATGLWFDPPLSMRLRSVEEL
TQFFLREIQYGIEDTGIRAGIIKVATTGKATPFQELVLKAAARASLATGVPVTTHTA
ASQRDGEQQAAIFESEGLSPSRVCIGHSDDTDDLSYLTALAARGYLIGLDHIPHSAI
GLEDNASASALLGIRSWQTRALLIKALIDQGYMKQILVSNDWLFGFSSYVTNIMDV
MDRVNPDGMAFIPLRVIPFLREKGVPQETLAGITVTNPARFLSPTLRAS

### REFERENCES

Afriat L., Roodveldt C., Manco G., and Tawfik D. S. The latent promiscuity of newly identified microbial lactonases is linked to a recently diverged phosphotriesterase. Biochemistry, 2006, 45, 13677-13686
Bassler B. L. How bacteria talk to each other: regulation of gene expression by quorum sensing. Current opinion in microbiology, 1999, 2, 582-587
Bhat K. A., Masood S. D., Bhat N. A., Bhat M. A., Razvi S. M., Mir M. R., Akhtar S., Wani N., and Habib M. Current status of post harvest soft rot in vegetables: a review. Asian Journal of Plant Sciences, 2010, 9, 200-208
Blachinsky D., Shtienberg D., Oppenheim D., Zilberstaine M., Levi S., Zamski E., Shoseyov O. The role of autumn infections in the progression of fire blight symptoms in perennial pear branches. Plant Dis., 2003
Cho C. M. H., Mulchandani A., Chen W. Functional analysis of organophosphorus hydrolase variants with high degradation activity towards organophosphate pesticides. Protein Engineering Design and Selection, 2006, 19, 99-105
Cho C. M. H., Mulchandani A., Chen W. Altering the substrate specificity of organophosphorus hydrolase for enhanced hydrolysis of chlorpyrifos. Applied and environmental microbiology, 2004, 70, 4681
Chow J. Y., Xue B., Lee K. H., Tung A., Wu L., Robinson, R. C., Yew W. S. Directed evolution of a thermostable quorum-quenching lactonase from the amidohydrolase superfamily. Journal of Biological Chemistry, 2010, 285, 40911-40920
Conway B. A., Greenberg E. P. Quorum-sensing signals and quorum-sensing genes in Burkholderia vietnamiensis. Journal of Bacteriology, 2002, 184, 1187
Cooper J., Dobson B. The benefits of pesticides to mankind and the environment. Crop Protection, 2007, 26, 1337-1348
Crépin A., Barbey C., Beury-Cirou A., Hélias V., Taupin L., Reverchon S., Nasser W., Faure D., Dufour A., Orange N., Feuilioley M., Heurlier K., Burini J. F., Latour X. Quorum sensing signaling molecules produced by reference and emerging soft-rot bacteria (Dickeya and Pectobacterium spp.). PLoS One, 2012a, 7, e35176
Crépin A., Beury-Cirou A., Barbey C., Farmer C., Hélias V., Burini J. F., Faure D., Latour, X. N-acyl homoserine lactones in diverse Pectobacterium and Dickeya plant pathogens: diversity, abundance, and involvement in virulence. Sensors, 2012b, 12, 3484-3497
Dong Y. J., Bartlam M., Sun L., Zhou Y. F., Zhang Z. P., Zhang C. G., Rao Z., Zhang X. E. Crystal structure of methyl parathion hydrolase from Pseudomonas sp. WBC-3. Journal of molecular biology, 2005, 353, 655-663.
Duerkop B. A., Varga J., Chandler J. R., Peterson S. B., Herman J. P., Churchill M. E., Parsek M. R., Nierman W. C., Greenberg E. P. Quorum-sensing control of antibiotic synthesis in Burkholderia thailandensis. Journal of bacteriology, 2009, 191, 3909
Freudl R. Signal peptides for recombinant protein secretion in bacterial expression systems. Microbial cell factories, 2018, 17, 1-10
Goldenzweig A., Goldsmith M., Hill S. E., Sussman J. L., Tawfik D. S., Fleishman S. J., Gertman O., Laurino P., Ashani Y., Dym O., Unger T., Albeck S., Prilusky J., Lieberman R. L., Aharoni A., Silman I. Automated structure- and sequence-based design of proteins for high bacterial expression and stability molecular cell technology automated structure- and sequence-based design of proteins for high bacterial expression and stability. Mol. Cell, 2016, 63, 1-10
Goldsmith M., Aggarwal N., Ashani Y., Jubran H., Greisen P. J., Ovchinnikov S., Leader H., Baker D., Sussman J. L., Goldenzweig A., Fleishman S. J., Tawfik D. S. (2017). Overcoming an optimization plateau in the directed evolution of highly efficient nerve agent bioscavengers. Protein Engineering, Design and Selection, 2017, 30, 333-345
Holm L., Sander C. An evolutionary treasure: unification of a broad set of amidohydrolases related to urease. Proteins: Structure, Function, and Bioinformatics, 1997, 28, 72-82
Homaei A. A., Sariri R., Vianello F., Stevanato R. Enzyme immobilization: An update. J. Chem. Biol., 2013
Jayaraman A., Wood T. K. Bacterial quorum sensing: signals, circuits, and implications for biofilms and disease. Annu. Rev. Biomed. Eng., 2008, 10, 145-167
Karthick A., Nallathambi G., Karthick A. S. A Review on Methods, Application and Properties of Immobilized Enzyme. Che Sci Rev Lett., 2012
Khadeja L., Grigoriants I., Halperin-Sternfeld M., Yona A., Adler-Abramovich L., Sonochemical Functionalization of Cotton and Non-Woven Fabrics with Bio-Inspired Self-Assembled Nanostructures. Isr. J. Chem., 2019
Khersonsky O., Tawfik D. S., Chromogenic and fluorogenic assays for the lactonase activity of serum paraoxonases. ChemBioChem, 2006, 7, 49-53
Lade H., Paul D., Kweon J. H. N-acyl homoserine lactone-mediated quorum sensing with special reference to use of quorum quenching bacteria in membrane biofouling control. BioMed research international, 2014
Li T., Wang D., Liu N., Ma Y., Ding T., Mei Y., Li J. Inhibition of quorum sensing-controlled virulence factors and biofilm formation in Pseudomonas fluorescens by cinnamaldehyde. International journal of food microbiology, 2018, 269, 98-106
Licciardello C., Bertani I., Steindler L., Bella P., Venturi V., Catara V. Pseudomonas corrugata contains a conserved N-acyl homoserine lactone quorum sensing system; its role in tomato pathogenicity and tobacco hypersensitivity response. FEMS microbiology ecology, 2007, 61, 222-234
Loh J., Pierson E. A., Pierson III L. S., Stacey G., Chatterjee A. Quorum sensing in plant-associated bacteria. Current opinion in plant biology, 2002, 5, 285-290
Lyagin L. V., Efremenko E. N. Biomolecular engineering of biocatalysts hydrolyzing neurotoxic organophosphates. Biochimie, 2018, 144, 115-121
Maisuria V. B., Nerurkar, A. S. Interference of quorum sensing by Delftia sp. VM4 depends on the activity of a novel N-acylhomoserine lactone-acylase. PloS one, 2015, 10, e0138034
Mandrich L., Merone L., Manco G. Hyperthermophilic phosphotriesterases/lactonases for the environment and human health. Environmental technology, 2010, 31, 1115-1127
Marchesan S., Vargiu A. V., Styan K. E. The Phe-Phe motif for peptide self-assembly in nanomedicine. Molecules. 2015
Miller M. B., Bassler B. L. Quorum sensing in bacteria. Annual Reviews in Microbiology, 2001, 55, 165-199
Ng T. K., Gahan L. R., Schenk C., Ollis D. L. Altering the substrate specificity of methyl parathion hydrolase with directed evolution. Archives of biochemistry and biophysics, 2015, 573, 59-68
Park B. W., Yoon D. Y., Kim D. S. Recent progress in bio-sensing techniques with encapsulated enzymes. Biosens. Bioelectron., 2010
Park B. W., Zheng R., Ko K. A., Cameron B. D., Yoon D. Y., Kim D. S. A novel glucose biosensor using bi-enzyme incorporated with peptide nanotubes. Biosens. Bioelectron., 2012a
Park B. W., Ko K. A., Yoon D. Y., Kim D. S. Enzyme activity assay for horseradish peroxidase encapsulated in peptide nanotubes. Enzyme Microb. Technol., 2012b
Persat A., Nadell C. D., Kim M. K., Ingremeau F., Siryaporn A., Drescher K., Wingreen N. S., Bassler B. L, Gitai Z., Stone H. A. The mechanical world of bacteria. Cell, 2015, 161, 988-997
Pieper U., Webb B. M., Dong G. Q., Schneidnan-Duhovny D., Fan H., Kim S. J. Khuri N., Spill Y. G., Weinkam P., Hammel M., Tainer J. A., Nilges M., Sali A. ModBase, a database of annotated comparative protein structure models and associated resources. Nucleic acids research, 2014, 42(D1), D336-D346
Poonguzhall P., Selvaraj S., Madhaiyan M., Sa T. Production of acyl-homoserine lactone quorum-sensing signals is wide-spread in gram-negative Methylobacterium. Journal of microbiology and biotechnology, 2007, 17, 226-233
Roodveldt C., Tawfik D. S. Shared promiscuous activities and evolutionary features in various members of the amidohydrolase superfamily. Biochemistry, 2005
Santander R. D., Biosca E. G. Erwinia amylovora psychotropic adaptations: Evidence of pathogenic potential and survival at temperate and low environmental temperatures. PeerJ., 2017
Schuster M., Greenberg E. P. A network of networks: quorum-sensing gene regulation in Pseudomonas aeruginosa. International journal of medical microbiology, 2006, 296, 73-81
Seibert C. M., Raushel F. M. Structural and catalytic diversity within the amidohydrolase superfamily. Biochemistry., 2005
Singh N., Golime R., Acharya J., Palit M. Quantitative Proteomic Changes after Organophosphorous Nerve Agent Exposure in the Rat Hippocampus. ACS Chemical Neuroscience, 2020, 11, 2638-2648
Tallat S., Hussien R., Mohamed R. H., Abd El Wahab M. B., Mahmoud M. Caspases as prognostic markers and mortality predictors in acute organophosphorus poisoning. Journal of Genetic Engineering and Biotechnology, 2020, 18, 1-8
Venturi V., Venuti C., Devescovi G., Lucchese C., Friscina A., Degrassi G., Aguilar C., Mazzucchi U. The plant pathogen Erwinia amylovora produces acyl-homoserine lactone signal molecules in vitro and in planta. FEMS microbiology letters, 2004a, 241, 179-183
Venturi V., Friscina A., Bertani I., Devescovi G., Aguilar C. Quorum sensing in the Burkholderia cepacia complex. Research in microbiology, 2004b, 155, 238-244.
Vrancken K., Holtappels M., Schoofs H., Deckers T., Valcke R. Pathogenicity and infection strategies of the fire blight pathogen Erwinia amylovora in Rosaceae: state of the art. Microbiology, 2013, 159(Pt_5), 823-832
Wang W. Z., Morohoshi T., Someya N., Ikeda T. AidC, a novel N-acylhomoserine lactonase from the potato root-associated Cytophaga-Flavobacteria-Bacteroides (CFB) group bacterium Chryseobacterium sp. strain StRB126. Applied and environmental microbiology, 2012, 78, 7985
Waters C. M., Bassler B. L. Quorum sensing: cell-to-cell communication in bacteria. Annu. Rev. Cell Dev. Biol., 2005, 21, 319-346.
Wierenga R. K. The TIM-barrel fold: a versatile framework for efficient enzymes. FEBS letters, 2001, 492, 193-198
Wilding M., Hong N., Spence M., Buckle A. M., Jackson C. J. Protein engineering: the potential of remote mutations. Biochemical Society Transactions, 2019, 47, 701-711
Zhang L., Wang H., Liu X., Zhou W., Rao Z. The crystal structure of the phosphotriesterase from M. tuberculosis, another member of phosphotriesterase-like lactonase family. Biochemical and biophysical research communications, 2019, 510, 224-229

1. A composition comprising a plurality of nano- or microspheres, and/or a plurality of tubular nanostructures, each encapsulating an esterase or a functional fragment thereof, wherein each one of said nano- and/or microspheres, or said tubular nanostructures, being formed of a plurality of aromatic dipeptides comprising end-capping modified aromatic dipeptides, non-modified aromatic dipeptides, or a combination thereof; and
said composition having a pH suitable for the activity of said esterase.

2. The composition of claim 1, wherein:
(i) said plurality of aromatic dipeptides comprises said end-capping modified aromatic dipeptides only, said non-modified aromatic dipeptides only, or a combination of said end-capping modified aromatic dipeptides and said non-modified aromatic dipeptides;
(ii) each one of said end-capping modified aromatic dipeptides is an aromatic dipeptide protected at an either amino- or carboxyl group thereof; or
(iii) said non-modified aromatic dipeptides and/or said end-capping modified aromatic dipeptides each is a homodipeptide.

3-4. (canceled)

5. The composition of claim 42, wherein:
(i) said plurality of aromatic dipeptides comprises a combination of said end-capping modified aromatic dipeptides and said non-modified aromatic dipeptides, and the molar ratio between said end-capping modified aromatic dipeptides and said non-modified aromatic dipeptides in said combination ranges from about 1:1 to about 1:100 respectively;
(ii) each one of said end-capping modified aromatic dipeptides is protected at the N-terminus thereof; or
(iii) each one of said non-modified aromatic dipeptides is phenylalanine-phenylalanine dipeptide; and/or each one of said end-capping modified aromatic dipeptides is an N-terminus modified phenylalanine-phenylalanine dipeptide.

6-7. (canceled)

8. The composition of claim 5, wherein each one of said end-capping modified aromatic dipeptides is protected with tert-butoxycarbonyl (Boc) or 9H-fluorenylmethyloxycarbonyl (Fmoc).

9-11. (canceled)

12. The composition of claim 1, wherein (i) said nano- or microspheres are characterized by a diameter in a range of from about 50 nm to about 2 micron; or (ii) said tubular nanostructures are characterized by a length in a range of from about 10 nm to about 2000 nm.

13. The composition of claim 1, having a pH in the range of from about 7 to about 11.

14. The composition of claim 1, further comprising ions of a metal capable of coordinating with, and which is required for enzymatic activity of, said esterase.

15. The composition of claim 14, wherein said ions are selected from the group consisting of divalent metal ions of manganese (Mn), zinc (Zn), cobalt (Co), cadmium (Cd), iron (Fe), nickel (Ni), calcium (Ca), magnesium (Mg), copper (Cu), and a combination thereof.

16. The composition of claim 1, wherein said esterase is a carboxylic ester hydrolase or a phosphoric triester hydrolase.

17. The composition of claim 1, wherein said 1,4-lactonase is a phosphotriesterase like lactonase (PLL).

18. The composition of claim 17, wherein:
(i) said PLL is the wild-type putative parathion hydrolase (PPH) from M. tuberculosis, having the amino acid sequence of SEQ ID NO: 1; or has a sequence comprising the amino acid sequence of SEQ ID NO: 1; or
(ii) said PLL has at least 30% identity to the wild-type PPH from M. tuberculosis having the amino acid sequence of SEQ ID NO: 1, and a TIM-barrel fold substantially identical to that of said wild-type PPH; and said PLL preserves the catalytic residues of said wild type PPH in its active site.

19. (canceled)

20. The composition of claim 18, wherein:
(i) said PLL is a mutant of said wild-type PPH, or a functional fragment thereof, in which the glycine residue at a position corresponding to position 59 of SEQ ID NO: 1 is substituted by an amino acid residue selected from the group consisting of valine, alanine, leucine, and isoleucine; and/or the histidine residue at a position corresponding to position 172 of SEQ ID NO: 1 is substituted by an amino acid residue selected from the group consisting of tyrosine, phenylalanine, and tryptophan; or
(ii) said PLL comprises an amino acid sequence selected from the group consisting of the amino acid sequences of SEQ ID NOs: 4-101.

21. The composition of claim 20, wherein in the sequence of said mutant, either (i) the glycine residue at a position corresponding to position 59 of SEQ ID NO: 1 is substituted by valine; or (ii) the histidine residue at a position corresponding to position 172 of SEQ ID NO: 1 is substituted by tyrosine.

22. The composition of claim 21, wherein said mutant comprises the amino acid sequence of SEQ ID NO: 2 or SEQ ID NO: 3.

23-25. (canceled)

26. The composition of claim 1, wherein:
(i) said aryldialkylphosphatase is the wild-type methyl parathion hydrolase (MPH) from Pseudomonas sp. WBC-3, having the amino acid sequence of SEQ ID NO: 102, or has a sequence comprising the amino acid sequence of SEQ ID NO: 102; or
(ii) said aryldialkylphosphatase has at least 70% identity to the wild-type MPH from Pseudomonas sp. WBC-3 having the amino acid sequence of SEQ ID NO: 102, and preserves the catalytic residues of said wild type MPH in its active site.

27. (canceled)

28. The composition of claim 26, wherein said aryldialkylphosphatase is a mutant of said wild-type MPH, or a functional fragment thereof.

29. The composition of claim 1, wherein:
(i) said aryldialkylphosphatase is the PTE from Pseudomonas diminuta, having the amino acid sequence of SEQ ID NO: 103, or has a sequence comprising the amino acid sequence of SEQ ID NO: 103; or
(ii) said aryldialkylphosphatase has at least 70% identity to the PTE from Pseudomonas diminuta having the amino acid sequence of SEQ ID NO: 103, and preserves the catalytic residues of said PTE in its active site.

30. (canceled)

31. The composition of claim 16, wherein said esterase or functional fragment thereof further comprises a tag.

32. The composition of claim 16, further comprising:
(i) a Tris buffer and divalent ions of Mn, wherein said esterase is a carboxylic ester hydrolase; or
(ii) a Tris buffer and divalent ions of Zn, wherein said esterase is a phosphoric triester hydrolase.

33. The composition of claim 1, wherein said composition is in dried form.

34. A plant, or a part, organ or plant propagation material thereof, at least partly covered or coated with a composition according to claim 16, wherein said esterase is a carboxylic ester hydrolase.

35. A method for treating or preventing an infection of a bacterium in a plant or a part, organ or plant propagation material thereof, being infected by or susceptible to a bacterium secreting a lactone selected from N-(3-hydroxybutanoyl)-L-homoserine lactone (C4-HSL), N-(3-oxo-hexanoyl)-homoserine lactone (C6-oxo-HSL), N-[(3S)-tetrahydro-2-oxo-3-furanyl]octanamide (C8-oxo-HSL), and N-[(3S)-tetrahydro-furanyl]decanamide (C10-HSL), said method comprising applying on said plant or said part, organ or plant propagation material thereof, a composition of claim 16, wherein said esterase is a carboxylic ester hydrolase.

36. The method of claim 35, wherein said bacterium is selected from the group consisting of Erwinia amylovora, Pectobacterium carotovorum, Pseudomonas syringae, Pseudomonas corrugata, Burkholderia vietnamiensis, Burkholderia cepacia, Burkholderia thailandensis and Pseudomonas aeruginosa, including any pathovars.

37. The method of claim 36, wherein said bacterium is Erwinia amylovora, Pectobacterium carotovorum, or Pseudomonas syringae.

38. A method for decomposing/degrading an organophosphorus compound from a media contaminated with said organophosphorus compound, said method comprising applying to said contaminated media a composition according to claim 16, wherein said esterase is a phosphoric triester hydrolase.

39. The method of claim 38, wherein said media is a soil, a produce, or a water source.

40. The method of claim 38, wherein said organophosphorus compound is a pesticide including a phosphate type organophosphorus pesticide;
a thiono type organophosphorus pesticide;
a thiol type organophosphorus pesticide;
and a dithiol type organophosphorus pesticide.

41. The composition of claim 16, wherein said carboxylic ester hydrolase is a 1,4-lactonase or a quorum-quenching N-acyl-homoserine lactonase; or said phosphoric triester hydrolase is an aryldialkylphosphatase.

42. The composition of claim 32, wherein said carboxylic ester hydrolase is a 1,4-lactonase or a quorum-quenching N-acyl-homoserine lactonase; or said phosphoric triester hydrolase is an aryldialkylphosphatase.

43. The plant, or a part, organ or plant propagation material thereof of claim 34, wherein said carboxylic ester hydrolase is a 1,4-lactonase or a quorum-quenching N-acyl-homoserine lactonase.

44. The method of claim 35, wherein said carboxylic ester hydrolase is a 1,4-lactonase or a quorum-quenching N-acyl-homoserine lactonase.

45. The method of claim 38, wherein said phosphoric triester hydrolase is an aryldialkylphosphatase.

46. The method of claim 40, wherein said phosphate type organophosphorus pesticide is paraoxon, methyl paraoxon, or dichlorvos; said thiono type organophosphorus pesticide is parathion, methyl parathion, or chlorpyrifos; and said thiol type organophosphorus pesticide is malathion or dimethoate.


SEQUENCE LISTING (103 sequences)

SEQ ID NO: 1 - protein, 330 aa, Mycobacterium tuberculosis
MISEFPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 2 - protein, 330 aa, Artificial Sequence
  synthetic
MISEFPELNTARGPIDTADLGVTLMHEHVFIMTTEIAQNYPEAWGDEDKRVAGAIARLVELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 3 - protein, 330 aa, Artificial Sequence
  synthetic
MISEFPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAYKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 4 - protein, 716 aa, Artificial Sequence
  synthetic
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 5 - protein, 716 aa, Artificial Sequence
  synthetic
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFPELNTARGPIDTADLGVTLMHEHVFIMTTEIAQNYPEAWGDEDKRVAGAIARLVELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 6 - protein, 715 aa, Artificial Sequence
  synthetic
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAYKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGY

SEQ ID NO: 7 - protein, 415 aa, Mycobacterium tuberculosis
MRSRPTKSRKSEYQIRSWSSCLSRAFPLDSKKSTVAAMISPERRSGYLRVKWLGLSSIRIGSRAVRSTTRVTLLHCGPARHRLRPSISDVPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 8 - protein, 415 aa, Mycobacterium tuberculosis
MRSRPTKSRKSEYQIRSWSSCLSRAFPLDSKKSTVAAMISPERRSGYLRVKWLGLSSIRIGSRAVRSTTRVTLLHCGPARHRLRPSISDVPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVELSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 9 - protein, 345 aa, Mycobacterium tuberculosis
MTLLHCGPARHRLRPSISDVPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 10 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 11 - protein, 326 aa, Mycobacterium tuberculosis
MPQLNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 12 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIMARMCERGHADKMVLSHDACCYFDALPEELVPVAMPNWHYLHIHNDVIPALKQHGVTDEQLHTMLVDNPRRIFERQGGYQ

SEQ ID NO: 13 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPAAMPNWHYLHIHNDVIPALKQHGVTDEQLHTMLVDNPRRIFERQGGYQ

SEQ ID NO: 14 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVELSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 15 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLFTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 16 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDAGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 17 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDLPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 18 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQAGYQ

SEQ ID NO: 19 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDQFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 20 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRANIVARMCERGHADKMVLSHDACCYFDALPEELVPVAMPNWHYLHIHNDVIPALKQHGVTDEQLHTMLVDNPRRIFERQGGYQ

SEQ ID NO: 21 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTNEQLHTMLVDNPRRIFERQGGYQ

SEQ ID NO: 22 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDTTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 23 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMADMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 24 - protein, 326 aa, Mycobacterium tuberculosis; identical to SEQ ID NO: 23

SEQ ID NO: 25 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGFYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 26 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGVPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 27 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLQIHNDVIPALKQHGVTDEQLHTMLVDNPRRIFERQGGYQ

SEQ ID NO: 28 - protein, 326 aa, Mycobacterium tuberculosis
MPELNTARGPIDTADLGVTLMHEHVFIMTTEIAQNYPEAWGDEDKRVAGAIARLGELKARGVDTIVDLKVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 29 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPLQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 30 - protein, 326 aa, Mycobacterium tuberculosis
MPELNTARGPIDTADLGVTLMHEHVLIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 31 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPLYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 32 - protein, 326 aa, Mycobacterium tuberculosis
MPELNTARGPIDTADLGVTLMHEHVFIMTTEIAQKYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 33 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTHNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 34 - protein, 326 aa, Mycobacterium tuberculosis
MPELNTARGPIDTADLGVMLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 35 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVGNPRRIFERQGGYQ

SEQ ID NO: 36 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQHIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 37 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIVERQGGYQ

SEQ ID NO: 38 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGRCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 39 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGRTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 40 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSSLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 41 - protein, 326 aa, Mycobacterium tuberculosis
  misc_feature (145)..(145): Xaa can be any naturally occurring amino acid
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILXCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 42 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDERGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 43 - protein, 325 aa, Mycobacterium tuberculosis
MPELNTARRPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGY

SEQ ID NO: 44 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRCIFERQGGYQ

SEQ ID NO: 45 - protein, 326 aa, Mycobacterium tuberculosis
MPELNTARGPIDTADLGVTLMHEHVFIMTTEIAQNYPEAWGDEDKRVAGAIARLGELKVRGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGVPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 46 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYKKVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 47 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCCFDALPEELVPVAMPNWHYLHIHNDVIPALKQHGVTDEQLHTMLVDNPRRIFERQGGYQ

SEQ ID NO: 48 - protein, 326 aa, Mycobacterium tuberculosis
MPELNTARGPIDTADLGVTLMHEHVFIMTTEIAQNYPEAWGDEDKRVAGAIARLGELKVRGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGVPISTHTHAGLRRGLDQQRIFAEEGVDLSRVIIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 49 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLCTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVELSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 50 - protein, 328 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVELSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFIFERQGGYQ

SEQ ID NO: 51 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYCDALPGELVPVAMPNWHYLHIHNDVIPALKQHGVTDEQLHTMLVDNPRRIFERQGGYQ

SEQ ID NO: 52 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTHKHVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 53 - protein, 327 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAKEGVELSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFIFERQGGY

SEQ ID NO: 54 - protein, 326 aa, Mycobacterium canettii
MPELNTARGPIDTADLGVTLMHEHVFIMTTEIAQNYPEAWGDEDKRVAGAIARLGELKVRGVDTIVDLTVIGLGRYIPRIARVAAATELNIVVATGLYTYNDVPFSFHYLGPGARLGGPEIMTDMFVRDIEHGIADTGIKAGILKCATDEPGLTPGVERVLRAVAQAHKRTGVPISTHTHAGLRRGLDQQRIFAEEGVDLSRVIIGHCGDSTDVGYLEELIAAGSYLGMDRFGVDVISPFQDRVNIVARMCERGHADKMVLSHDACCYFDALPEELVPVAMPNWHYLHIHNDVIPALKQRGVTDEQLHTMLVDNPRRIFERHGGYQ

SEQ ID NO: 55 - protein, 326 aa, Mycobacterium tuberculosis
  misc_feature (75)..(75): Xaa can be any naturally occurring amino acid
  misc_feature (82)..(84): Xaa can be any naturally occurring amino acid
  misc_feature (96)..(96): Xaa can be any naturally occurring amino acid
  misc_feature (183)..(185): Xaa can be any naturally occurring amino acid
  misc_feature (196)..(196): Xaa can be any naturally occurring amino acid
  misc_feature (212)..(212): Xaa can be any naturally occurring amino acid
  misc_feature (310)..(310): Xaa can be any naturally occurring amino acid
MPELNTARGPIDTADLGVTLMHEHVFIMTTEIAQNYPEAWGDEDKRVAGAIARLGELKARGVDTIVDLTVIGLGXYIPRIAXXXAATELNIVVATXLYTYNDVPFYFHYLGPGAQLDGPEIMTDMFVRDIEHGIADTGIKAGILKCATDEPGLTPGVERVLRAVAQAHKRTGAPISTHTHAGXXXGLDQQRIFAEXGVDLSRVVIGHCGDSXDVGYLEELIAAGSYLGMDRFGVDVISPFQDRVNIVARMCERGHADKMVLSHDACCYFDALPEELVPVAMPNWHYLHIHNDVIPALKQHGVTDEQLHTXLVDNPRRIFERQGGYQ

SEQ ID NO: 56 - protein, 306 aa, Mycobacterium tuberculosis
MHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 57 - protein, 313 aa, Mycobacterium tuberculosis
MVGDRDRVLEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 58 - protein, 325 aa, Mycobacterium  lacus
MPVLNTARGPIDTADLGVTLMHEHVFIMTTEIAQNYPEAWGDEERRVADAITRLGELKARGVDTIVDLTVIGLGRYIPRIARVASATELNIVVATGLYTYNDVPFYFHYLGPGGMLDGPEIMTDMFVRDIEQGIADTGIKAGILKCATDEPGVTPGVERVLRAVAQAHKRTGVPISTHTHAGLRRGLEQQRIFAEEGVDLSRVIIGHSGDSTDVGYLEELIANGSYLGMDRFGIDVISPFEDRVNIVAQMCERGHADKMVLSHDANCYFDALPEELVAVAAPNWHYLHIHNDVIPALKERGVTDEQLHTMLVDNPRRIFERQGGY

SEQ ID NO: 59 - protein, 326 aa, Mycobacterium avium
MSELNTARGPIDTAALGVTLMHEHVFIMTTEIAQNYPEAWGDEEQRVADAIDRLNELKARGVDTIVDLTVIGLGRYIPRIARVAAATELNIVVATGLYTYNDVPFYFHYLGPGAELGGPEIMTDMFVRDIEQGIADTGIKAGILKCATDAPGVTPGVERVLRAVAQAHKRTGVPISTHTHAGLRRGLDQQRIFEEEGVDLSRVVIGHSGDSTDVGYLEELIAAGSYLGMDRFGIDAISPFEDRVNIVAQMCERGHADKMVLSHDANCYFDALPEELVPQVMPNWHYLHIHNDVIPALKQRGVTDEQLHTMLVDNPRRIFERQGPYG

SEQ ID NO: 60 - protein, 299 aa, Mycobacterium tuberculosis
MTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 61 - protein, 326 aa, Mycobacterium haemophilum
MSELNTARGPISTADLGVTLMHEHVFIMTTEIAQNYPEAWGDEEQRVADAIARLSELKARGVDTIVDLTVIGLGRYIPRIARVAAATELNIVVATGLYTYNDVPFYFHYLGPGAPLNGPEIMTDMFVRDIEQGVADTGIKAGILKCATDEPGITPGVERVLRAVAQAHKRTGVPISTHTHAGLRRGLEQQRIFAEEGVDLSRVIIGHSGDSTDVGYLEELIANGSYLGMDRFGVDVILPFEDRVSIVAQMCERGHADKMVLSHDANCYFDALPEALQPVAAPNWHYLHIHNDVIPALKERGVTDEQLHTMLVDNPRRIFERQGTYE

SEQ ID NO: 62 - protein, 326 aa, Mycobacterium  sp.
MSELNTARGPIDTADLGVTLMHEHVFIMTTEVVQNYPEAWGDEDKRVADAVARLNELKSRGVDTIVDLTVIGLGRYIPRIARVAAQTELNIVVATGLYTYNDVPFCFHYLGPGTQLDGPETMTDLFVRDIEQGIADTGIKAGILKCATDEPGITPGVERVLRAVAQAHKRTGVPISTHTHAGLRRGLEQQRIFEEEGVDLSRVVIGHSGDSTDVGYLEELVAAGSYLGMDRFGLDVILPFEDRVNIVAQMCERGHADKMVLSHDASCYFDALPEALLPVAAPNWNYLHIHNDVIPALKQRGVTDEQLHTMLVDNPRRIFERQGGYE

SEQ ID NO: 63 - protein, 326 aa, Mycobacterium shimoidei
MSQLNTARGPIDTADLGVTLMHEHVFIMTTEVAQNYPEAWGDEEQRVADAITRLNELKARGVDTIVDLTVIGLGRYIPRIARVAAATELNIVVATGLYTYNDVPFRFHYQGPGTQLDGPEIMTDMFVRDIEQGIADTGIKAAILKCATDEPGITPGVERVLRAVAQAHKRTGVPISTHTHAGLRRGLEQQRIFEEEGADLSRVIIGHSGDSTDVGYLEELIAAGSYLGMDRFGIDVISPFEDRVNIVARMCERGHADKMVLSHDANCYFDALPEELLPVVAPNWHYLHIHNDVIPALKERGVTDEQLHTMLVDNPRRIFERTGAYE

SEQ ID NO: 64 - protein, 326 aa, Mycobacterium shimoidei
MSQLNTARGPIDTADLGVTLMHEHVFIMTTEVAQNYPEAWGDEEQRVADAITRLNELKARGVDTIVDLTVIGLGRYIPRIARVAAATELNIVVATGLYTYNDVPFRFHYQGPGTQLDGPEIMTDMFVRDIEQGIADTGIKAAILKCATDEPGITPGVERVLRAVAQAHKRTGVPISTHTHAGLRRGLEQQRIFEEEGVDLSRVIIGHSGDSTDVGYLEELIAAGSYLGMDRFGIDVISPFEDRVNIVAQMCERGHADKMVLSHDANCYFDALPEELLPVVAPNWHYLHIHNDVIPALKERGVTDEQLHTMLVDNPRRIFERTGAYE

SEQ ID NO: 65 - protein, 325 aa, Mycobacterium bohemicum
MSELNTARGPIDTADLGVTLMHEHVFIMTTEIAQNYPEAWGDEDQRVADAITRLNELKARGVDTIVDLTVIGLGRYIPRIARVAAATELNIVVATGLYTYNDVPFCFHYLGPGAALGGPEVMTDMFVRDIEQGIADTGVKAGILKCATDEPGITPGVERVLRAVAQAHRRTGVPISTHTHAGLRRGLEQQRIFEEEGVDLSRVVIGHSGDSTDVGYLEELIAAGSYLGMDRFGIDVISPFEDRVNIVAQMCERGHADKMVLSHDANCYFDALPEELLAVAAPNWHYLHIHNDVIPALKERGVTDEQLHTMLVDNPRRIFERRGAY

SEQ ID NO: 66 - protein, 325 aa, Mycobacterium kansasii
MSELNTARGPIDTADLGVTLMHEHVFIMTTEVVQNYPEAWGDEDKRVADAVARLNELKSRGVDTIVDLTVIGLGRYIPRIARVAAQTELNIVVATGLYTYNDVPFCFHYLGPGTQLDGPETMTDLFVRDIEQGIADTGIKAGILKCATDDPGITPGVERVLRAVAQAHKRTGVPISTHTHAGLRRGLEQQRIFEEEGVDLSRVVIGHSGDSTDVGYLEELVAAGSYLGMDRFGLDVILPFEDRVNIVAQMCERGHADKMVLSHDASCYFDALPEALLPVAAPNWNYLHIHNDVIPALKQRGVTDEQLHTMLVDNPRRIFERQGGY

SEQ ID NO: 67 - protein, 326 aa, Mycobacterium avium
MSELNTARGPIDTAALGVTLMHEHVFIMTTEIAQNYPEAWGDEEQRVADAIDRLNELKARGVDTIVDLTVIGLGRYIPRIARVAAATELNIVVATGLYTYNDVPFCFHYLGPGAELGGPEIMTDMFVRDIEQGIADTGIKAGILKCATDAPGVTPGVERVLRAVAQAHKRTGVPISTHTHAGLRRGLDQQRIFEEEGVDLSRVVIGHSGDSTDVGYLEELIAAGSYLGMDRFGIDAISPFEDRVNIVAQMCERGHADKMVLSHDANCYFDALPEELVPQVMPNWHYLHIHNDVIPALKQRGVTDEQLHTMLVDNPRRIFERQGPYG

SEQ ID NO: 68 - protein, 335 aa, Mycobacterium sp.
MGYVSQPGIASELNTARGPIDTADLGVTLMHEHVFIMTTEIALNYPEAWGDEEKRVADAVTRLNELKARGVDTIVDLTVIGLGRYIPRIAKVAAATELNIVVATGLYTYNDVPFCFHYMGPGAQLGGPEIMTDMFVRDIEQGIADTGIKAGILKCATDEPGITPGVERVLRAVAQAHKRTGVPISTHTHAGLRRGLEQQRIFEEEGVDLSRVIIGHSGDSTDVGYLEELIAAGSYLGMDRFGIDVILPFEERVNIVATMCERGHADKMVLSHDANCYFDALPEEMLAVAAPNWHYLHIHNDVIPALKQRGVTDEQLHTMLVDNPRRIFERQGAYQ

SEQ ID NO: 69 - protein, 325 aa, Mycobacterium sp.
MSELNTARGPIDTADLGVTLMHEHVFIMTTEIAQNYPEAWGDEEKRVADAITRLNELKSRGVHTIVDLTVIGLGRYIPRIARVAAATELNIVVATGLYTYNDVPFRFHYQGPGCMLEGPEIMTDMFVRDIEQGIADTGVKAGILKCATDEPGLTPGVERVLRAVAQAHKRTGVPISTHTHAGLRRGLEQQRVFAEEGVDLSRVIIGHSGDSTDVGYLEELIAAGSYLGMDRFGIDLILPFEDRVNIVATMCERGHADKMVLSHDANCYFDALPEELVPVATPNWHYLHIHNDVIPALKERGVTDEQVHTMLVDNPRRIFERQGGY

SEQ ID NO: 70 - protein, 325 aa, Mycobacterium haemophilum
MSELNTARGPISTADLGVTLMHEHVFIMTTEIAQNYPEAWGDEEQRVADAIARLSELKARGVDTIVDLTVIGLGRYIPRIARVAAATELNIVVATGLYTYNDVPFYFHYLGPGAPLNGPEIMTDMFVRDIEQGVADTGIKAGILKCATDEPGITPGVERVLRAVAQAHKRTGVPISTHTHAGLRRGLEQQRIFAEEGVDLSRVIIGHSGDSTDVGYLEELIANGSYLGMDRFGVDVILPFEDRVSIVAQMCERGHAGKMVLSHDANCYFDALPEALQPVAAPNWHYLHIHNDVIPALKERGVTDEQLHTMLVDNPRRIFERQGTY

SEQ ID NO: 71 - protein, 326 aa, Mycobacterium persicum
MSELNTARGPIDTADLGVTLMHEHVFIMTTEVAQNYPEAWGDEDRRVADAVARLNELKSRGVDTIVDLTVIGLGRYVPRIARVAAQTELNIVVATGLYTYNDVPFCFHYLGPGTQLDGPETMTDLFVRDIEQGIADTGIKAGILKCATDEPGITPGVERVLRAVAQAHKRTGVPISTHTHAGLRRGLEQQRIFEEEGVDLSRVVIGHSGDSTDVGYLEELIAAGSYLGMDRFGLDVILPFEERVAIVAQMCERGHADKMVLSHDATCYFDALPEALLPVAAPNWHYLHIHNDVIPALKQRGVTDEQLHTMLVDNPRRIFERQGGYD

SEQ ID NO: 72 - protein, 326 aa, Mycobacterium persicum
MSELNTARGPIDTADLGVTLMHEHVFIMTTEVAQNYPEAWGDEDRRVADAVARLNELKSRGVDTIVDLTVIGLGRYVPRIARVAAETELNIVVATGLYTYNDVPFCFHYLGPGTQLDGPETMTDLFVRDIEQGIADTGIKAGILKCATDEPGITPGVERVLRAVAQAHKRTGVPISTHTHAGLRRGLEQQRIFEEEGVDLSRVVIGHSGDSTDVGYLEELIAAGSYLGMDRFGLDVILPFEERVAIVAQMCERGHADKMVLSHDATCYFDALPEALLPVAAPNWHYLHIHNDVIPALKQRGVTDEQLHTMLVDNPRRIFERQGGYD

SEQ ID NO: 73 - protein, 326 aa, Mycobacterium sp.
MSELNTARGPIDTADLGVTLMHEHVFIMTTEVVQNYPDAWGDEDKRVADAVARLNELKSRGVDTIVDLTVIGLGRYIPRIARVAAQTELNIVVATGLYTYNDVPFCFHYLGPGTQLDGPETMTDLFVRDIEQGIADTGIKAGILKCATDEPGITPGVERVLRAVAQAHKRTGVPISTHTHAGLRRGLEQQRIFEEEGVDLSRVVIGHSGDSTDVGYLEELIAAGSYLGMDRFGLDVILPFEDRVNIVAQMCERGHADKMVLSHDANCYFDALPEALLPVAAPNWNYLHIHNDVIPALKQRGVTDEQLHTMLVDNPRRIFERQGAYE

SEQ ID NO: 74 - protein, 326 aa, Mycobacterium sp.
MSELNTARGPIDTADLGVTLMHEHVFIMTTEIALNYPEAWGDEEKRVADAVARLNELKARGVDTIVDLTVIGLGRYIPRIAKVAASTDLNIVVATGLYTYNDVPFCFHYMGPGAQLGGPEIMTDMFVRDIEQGIADTGIKAGILKCATDEPGITPGVERVLRAVAQAHKRTGVPISTHTHAGLRRGLEQQRIFEEEGVDLSRVIIGHSGDSTDVGYLEELIAAGSYLGMDRFGIDVILPFEERVNIVATMCERGHADKMVLSHDANCYFDALPEEMLAVAAPNWHYLHIHNDVIPALKQRGVTDEQLHTMLVDNPRRIFERQGAYQ

SEQ ID NO: 75 - protein, 326 aa, Mycobacterium avium
MSELNTARGPIDTAALGVTLMHEHVFIMTTEIAQNYPEAWGDEEQRVADAIDRLNELKARGVDTIVDLTVIGRGRYIPRIARVAAATELNIVVATGLYTYNDVPFCFHYLGPGAELGGPEIMTDMFVRDIEQGIADTGIKAGILKCATDAPGVTPGVERVLRAVAQAHKRTGVPISTHTHAGLRRGLDQQRIFEEEGVDLSRVVIGHSGDSTDVGYLEELIAAGSYLGMDRFGIDAISPFEDRVNIVAQMCERGHADKMVLSHDANCYFDALPEELVPQVMPNWHYLHIHNDVIPALKQRGVTDEQLHTMLVDNPRRIFERQGPYG

SEQ ID NO: 76 - protein, 326 aa, Mycobacterium  interjectum
MSELNTARGAIDTADLGVTLMHEHVFIMTTEIALNYPEAWGDEEQRVADAIDRLNELKARGVDTIVDLTVIGLGRYIPRIARVAAATELNIVVATGLYTYNDVPFRFHYQGPGAPLDGPEIMTDMFVRDIEEGIADTGVKAGILKCATDEPGVTPGVERVLRAVAQAHKRTGVPISTHTHAGLRRGLEQQRIFSEEGVDLSRVVIGHSGDTTDVGYLEELIAAGSYLGMDRFGIDLISPFEDRVNIVATMCERGHADKMVLSHDANCYFDALPEELLPVAAPNWHYLHIHNDVIPALKERGVTDEQLHTMLVDNPRRIFERQGAYE

SEQ ID NO: 77 - protein, 326 aa, Mycobacterium sp.
MSELNTARGLIDTADLGVTLMHEHVFIMTTEVVQNYPEAWGDEDQRVADAVARLNELKARGVDTIVDLTVIGLGRYIPRIARVAAQTELNIVVATGLYTYNDVPFCFHYLGPGTQLDGPETMTDLFVRDIEQGIADTGIKAGILKCATDEPGITPGVERVLRAVAQAHKRTGVPISTHTHAGLRRGLEQQRIFEEEGVDLSRVVIGHSGDSTDVGYLEELIAAGSYLGMDRFGLDVILPFEDRVNIVARMCERGHADKMVLSHDATCYFDALPEALLPVAAPNWNYLHIHNDVIPALKQRGVTDEQLHTMLVDNPQRIFERQGAYE

SEQ ID NO: 78 - protein, 326 aa, Mycobacterium persicum
MSELNTARGPIDTADLGVTLMHEHVFIMTTEVAQNYPEAWGDEDRRVADAVARLNELKSRGGDTIVDLTVIGLGRYVPRIARVAAQTELNIVVATGLYTYNDVPFCFHYLGPGTQLDGPETMTDLFVRDIEQGIADTGIKAGILKCATDEPGITPGVERVLRAVAQAHKRTGVPISTHTHAGLRRGLEQQRIFEEEGVDLSRVVIGHSGDSTDVGYLEELIAAGSYLGMDRFGLDVILPFEERVAIVAQMCERGHADKMVLSHDATCYFDALPEALLPVAAPNWHYLHIHNDVIPALKQRGVTDEQLHTMLVDNPRRIFERQGGYD

SEQ ID NO: 79 - protein, 326 aa, Mycobacterium kansasii
MSELNPARGPIDTADLGVTLMHEHVFIMTTEVVQNYPDAWGDEDKRVADAVARLNELKSRGVDTIVDLTVIGLGRYIPRIARVAAQTELNIVVATGLYTYNDVPFCFHYLGPGTQLDGPETMTDLFVRDIEQGIADTGIKAGILKCATDEPGITPGVERVLRAVAQAHKRTGVPISTHTHAGLRRGLEQQRIFEEEGVDLSRVVIGHSGDSTDVGYLEELIAAGSYLGMDRFGLDVILPFEDRVNIVAQMCERGHADKMVLSHDANCYFDALPEALLPVAAPNWNYLHIHNDVIPALKQRGVTDEQLHTMLVDNPRRIFERQGAYE

SEQ ID NO: 80 - protein, 326 aa, Mycobacterium fragae
MSQLNTARGPIDTGDLGVTLMHEHVFIMTTEIALNYPEAWGDEDQRVSDAVNRLNELKSRGVDTIVDLTVIGLGRYIPRIARVAAETDLNIVVATGLYTYNDVPFRFHYQGPGTQLDGPEIMTDMFVRDIEEGIADTGIKAGILKCATDEPGITPGVERVLRAVAQAHKRTGVPISTHTHAGLRRGLEQQRIFEEEGVDLSRVIIGHSGDSTDIGYLEELIAAGSYLGMDRFGIDVILPFEDRVNIVAQMCERGHADKMVLSHDANCYFDALPEELMPVAAPNWHYLHIHNDVIPALKERGVTDEQLHTMLVDNPRRIFERQGAYQ

SEQ ID NO: 81 - protein, 326 aa, Mycobacterium kubicae
MSELNTARGPIDTANLGVTLMHEHVFIMTTEIIQNYPEAWGDEEKRVADAVARLNELKSRGVDSIVDLTVIGLGRYIPRIARVAAQTELNIVVATGIYTYNDVPFHFHYSGPGTILNGPEVMVDMFVRDIEQGIADTGVKAGILKCATDEPGLTPGVERVLRAVAQAHKRTGVPISTHTHAGLRRGLDQQRIFEEEGVDLSRVVIGHSGDSTDVGYLEELIASGSYLGMDRFGLDLISPFEDRVNIVAQMCERGHADKMVLSHDANCYFDALPEEVVPQVLPNWHYLHIHNDVIPALKERGVTDEQLHTMLVDNPRRIFERQGGYE

SEQ ID NO: 82 - protein, 326 aa, Mycobacterium kubicae
MSELNTARGPIDTANLGVTLMHEHVFIMTTEIIQNYPEAWGDEEKRVADAVARLNELKSRGVDSIVDLTVIGLGRYIPRIARVAAQTELNIVVATGIYTYNDVPFHFHYSGPGTILNGPEVMVDMFVRDIEQGIADTGVKAGILKCATDEPGLTPGVERVLRAVAQAHKRTGVPISTHTHAGLRRGLDQQRIFEEEGVDLSRVVIGHSGDSTDIGYLEELIASGSYLGMDRFGLDLISPFEDRVNIVAQMCERGHADKMVLSHDANCYFDALPEEVVPQVLPNWHYLHIHNDVIPALKERGVTDEQLHTMLVDNPRRIFERQGGYE

SEQ ID NO: 83 - protein, 326 aa, Mycobacterium sp. E3198
MSELNTARGAIDTADLGVTLMHEHVFIMTTEIALNYPEAWGDEEQRVADAIDRLNELKARGVDTIVDLTVIGLGRYIPRIARVAAATELNIVVATGLYTYNDVPFRFHYQGPGAPLDGPEIMTDMFVRDIEEGIADTGVKAGILKCATDEPGVTPGVERVLRAVAQAHKRTGVPISTHTHAGLRRGLEQQRIFESEGVDLSRVVIGHSGDSTDVGYLEELIAAGSYLGMDRFGIDLISPFEDRVNIVATMCERGHADKMVLSHDANCYFDALPEELLPVAAPNWHYLHIHNDVIPALKERGVTDEQLHTMLVDNPRRIFERQGAYA

SEQ ID NO: 84 - protein, 326 aa, Mycobacterium decipiens
MSALNTARGPIDTADLGVTLMHEHVFIMTTEIAQNYPEGWGDEEQRVADAITRLSELKARGVDTIVDLTVIGLGRYIPRIARVAAATELNIVVATGLYTYNDVPFFFHYLGPGGMLDGPEIMTDMFVRDIEEGIADTGIKAGILKCATDEPGITPGVERVLRAVAQAHMRTGVPISTHTHAGLRRGLEQQRIFAEEGVDLSRVIIGHSGDSTDVGYLEELISNGSYLGMDRFGIDLILPFEERVNIVARLCERGHADKMVLSHDANCYFDALPEALVPAAAPNWHYLHIHNDVIPALKERGVTDEQVRTMLVDNPRRIFERQGGYQ

SEQ ID NO: 85 - protein, 325 aa, Mycobacterium sp.
MSELNTARGSIDTADLGVTLMHEHVFIMTTEIALNYPEAWGDEEQRVADAITRLNELKARGVDTIVDLTVIGLGRYIPRIARVAAATELNIVVATGLYTYNDVPFCFHYMGPGAQLGGPEIMTEMFVRDIEQGIADTGIKAGILKCATDEPGITPGVERVLRAVAQAHKQTGVPISTHTHAGLRRGLEQQRIFEEEGVDLSRVIIGHSGDSTDVGYLEELISAGSYLGMDRFGIDVILPFEDRVNIVATMCERGHADKMVLSHDANCYFDALPEELLPVAAPNWHYLHIHNDVIPALKERGVTDEQLHTMLVDNPRRIFERQGSY

SEQ ID NO: 86 - protein, 325 aa, Mycobacterium sp. E342
MSELNTARGSIDTAQLGVTLMHEHVFIMTTEIAENYPEAWGDEERRVADAIDRLNELKSRGVDTIVDLTVIGLGRYIPRIARVAAATELNIVVATGLYTYNDVPFYFHYLGPGAELGGPEIMTDMFVRDIEQGIADTGVKAGILKCATDAPGVTPGVERVLRAVAQAHKRTGVPISTHTHAGLRRGLEQQRIFEEEGVDLSRVVIGHSGDSTDVGYLEELIAAGSYLGMDRFGIDAISPFEDRVNIVAQMCERGHADKMVLSHDANCYFDALPEELVPQVMPNWHYLHIHNDVIPALKERGVTDEQLHTMLVDNPRRIFEQTGAY

SEQ ID NO: 87 - protein, 325 aa, Mycobacterium sp. 852002-10029_SCH5224772; identical to SEQ ID NO: 86

SEQ ID NO: 88 - protein, 326 aa, Mycobacterium gastri
MSELNTARGPIDTADLGVTLMHEHVFIMTTEVVQNYPDAWGDEDKRVADAVGRLNVLKSRGVDTIVDLTVIGLGRYIPRIARVAAQTELNIVVATGLYTYNDVPFCFHYLGPGTQLDGPEIMTDLFVRDIEQGIADTGIKAGILKCATDEPGITPGVERVLRAVAQAHKRTGVPISTHTHAGLRRGLEQQRIFEEEGVDLSRVIIGHSGDSTDVGYLEELVAAGSYLGMDRFGIDVILPFEDRVNIVATMCERGHADKMVLSHDANCYFDALPEALLPVAAPNWHYLHIHNDVIPALTQRGVTDEQLHTMLVDNPRRIFERQGAYE

SEQ ID NO: 89 - protein, 325 aa, Mycobacterium riyadhense
MSELNTARGTIDTADLGVTLMHEHVFIMTTEIAQNYPEGWGDEEKRVADAITRLNELKARGVDTIVDLTVIGLGRYIPRIAQVAAQTELNIVVATGLYTYNDVPFCFHFMGPGGMLDGPEIMTDMFVRDIEQGIADTGIKAAILKCATDEPGVTPGVERVLRAVAQAHKRTGVPISTHTHAGLRRGLEQQRIFEEEGVDLSRVIIGHSGDSTDVGYLEELIAAGSYLGMDRFGVDVISPFEDRVNIVAQMCERGHADKMVLSHDANCYFDALPEALVPVVTPNWHYLHIHNDVIPALKQRGVTDEQVQTMLVDNPRRIFERQGGY

SEQ ID NO: 90 - protein, 326 aa, Mycobacterium nebraskense
MSELNTARGPIDTADLGVTLMHEHVFIMTTEIALNYPEAWGDEEKRVADAIARLNELKARGVDTIVDLTVIGLGRYIPRIARVAAATELNIVVATGLYTYNDVPFRFHYEGPGGMLDGPEIMTEMFVRDIEQGIADTGVKAGILKCATDEPGITPGVERVLRAVAQAHKRTGVPISTHTHAGLRRGLEQQRIFEEEGVDLTRVVIGHSGDSTDVGYLEELVAAGSYLGMDRFGLDVISPFEERVGIVATMCERGHADKMVLSHDANCYFDALPEELVPQMAPNWHYLHIHNDVIPALKERGVTDEQLHTMLVDNPRRIFERQGAYE

SEQ ID NO: 91 - protein, 326 aa, Mycobacterium kansasii
MSELNTARGPIDTADLGVTLMHEHVFIMTTEVVQNYPDAWGDEDKRVADAVARLNELKSRGVDTIVDLTVIGLGRYIPRIARVAAQTELNIVVATGLYTYNDVPFCFHYLGPGTQLDGPETMTDLFVRDIEQGIADTGIKAGILKCATDEPGITPGVERVLRAVAQAHKHTGVPISTHTHAGLRRGLEQQRIFEEEGVDLSRVVIGHSGDSTHVGYLEELIAAGSYLGMDRFGLDVILPFEDRVNIVAQMCERGHADKMVLSHDANCYFDALPEALLPVAAPNWNYLHIHNDVIPALKQRGVTDEQLHTMLVDNPRRIFERQGAYE

SEQ ID NO: 92 - protein, 326 aa, Mycobacterium sp. 852002-40037_SCH5390672
MSELNTARGPIDTADLGVTLMHEHVFIMTTEIAQNYPEAWGDEEKRVADAIARLNELKARGVDTIVDLTVIGLGRYIPRIARVAAATELNIVVATGLYTYNDIPFRFHYEGPGGMLGGPEIMTDMFVKDIEVGIADTGVKAGILKCATDEPGITPGVERVLRAVAQAHKRTGVPISTHTHAGLRRGLEQQRIFEEEGVDLTRVVIGHSGDSTDIGYLEELIAAGSYLGMDRFGLDVISPFEERVRIVAEMCERGHADKMVLSHDANCYFDALPEELVPQMAPNWHYLHIHNDVIPALKERGVTDEQLHTMLVDNPRRIFERQGSYE

SEQ ID NO: 93 - protein, 326 aa, Mycobacterium  kubicae
MSELNTARGPIDTANLGVTLMHEHVFIMTTEIIQNYPEAWGDEEKRVADAVARLNELKSRGVDSIVDLTVIGLGRYIPRIARVAAQTDLNIVVATGIYTYNDVPFHFHYNGPGTILNGPEVMVDMFVRDIEQGIADTGVKAGILKCATDEPGLTPGVERVLRAVAQAHKRTGVPISTHTHAGLRRGLDQQRIFEEEGVDLSRVVIGHSGDSTDIGYLEELIASGSYLGMDRFGLDLISPFEDRVNIVAQMCERGHADKMVLSHDANCYFDALPEEVVPQVLPNWHYLHIHNDVIPALKERGVTDEQLHTMLVDNPRRIFERQGGYE

SEQ ID NO: 94 - protein, 326 aa, Mycobacterium  marinum
MSALNTARGPIDTADLGVTLMHEHVFIMTTEVAQNYPEAWGDEEKRVADAITRLNELKSRGVDTIVDLTVIGLGRYIPRIARIAAATDLNIVVATGLYTYNDVPYRFHYQGPGGMLDGPEIMTDMFVRDIEGGIADTGVKAGILKCATDEPGITPGVERVLRAVAQTHKRTGVPISTHTHAGLRRGLEQQRIFEEEGVDLSRVIIGHSGDSTDVGYLEELIAAGSYLGMDRFGIDLILPFEDRVNIVATMCERGHADKMVLSHDANCYFDALPEELTAVATPNWHYLHIHNDVIPALKERGVTDEQVHTMLVDNPRRIFERQGGYQ

SEQ ID NO: 95 - protein, 326 aa, Mycobacterium malmoense
MSELNTARGPIDTADLGVTLMHEHVFIMTTEIALNYPEAWGDEDRRVADAIDRLNELKSRGVDTIVDLTVIGLGRYIPRIARVAAATELNIVVATGLYTYNDVPFRFHYEGPGGMLDGPEIMTEMFVRDIEQGIADTGIKAGILKCATDEPGITPGVERVLRAVAQAHKRTGVPISTHTHAGLRRGLEQQRIFEEEGVDLSRVVIGHSGDSTDVGYLEELIAAGSYLGMDRFGLDVISPFEERVKIVATMCERGHADKMVLSHDANCYFDALPEDLVPQMAPNWHYLHIHNDVIPALKERGVTDEQLHTMLVDNPRRIFERQGAYE

SEQ ID NO: 96 - protein, 326 aa, Mycobacterium scrofulaceum
MSELNTARGPIDTADLGVTLMHEHVFIMTTEIALNYPEAWGDEDRRVADAIDRLNELKSRGVDTIVDLTVIGLGRYIPRIARVAAATELNIVVATGLYTYNDVPFRFHYEGPGGLLDGPEIMTEMFVRDIEQGIADTGIKAGILKCATDEPGITPGVERVLRAVAQAHKRTGVPISTHTHAGLRRGLEQQRIFEEEGVDLSRVVIGHSGDSTDVGYLEELIAAGSYLGMDRFGLDVISPFEERVKIVATMCERGHADKMVLSHDANCYFDALPEDLVPQMAPNWHYLHIHNDVIPALKERGVTDEQLHTMLVDNPRRIFERQGAYE

SEQ ID NO: 97 - protein, 326 aa, Mycobacterium marinum
MSALNTARGPIDTADLGVTLMHEHVFIMTTEVAQNYPEAWGDEEKRVADAITRLNELKSRGVDTIVDLTVIGLGRYIPRIARIAAATDLNIVVATGLYTYNDVPYRFHYQGPGGMLDGPEIMTDMFVRDIEQGIADTGVKAGILKCATDEPGITPGVERVLRAVAQTHKRTGVPISTHTHAGLRRGLEQQRIFEEEGVDLSRVIIGHSGDSTDVGYLEELIAAGSYLGMDRFGIDLILPFEDRVSIVATMCERGHADKMVLSHDANCYFDALPEELTAVATPNWHYLHIHNDVIPALKERGVTDEQVHTMLVDNPRRIFERQGGYQ

SEQ ID NO: 98 - protein, 325 aa, Mycobacterium sp. E2497
MLELNTARGPIDTADLGVTLMHEHVFIMTTEIALNYPEAWGDEEKRVADAIARLNELKARGVDTIVDLTVIGLGRYIPRIARVAAATDLNIVVATGLYTYNDIPFRFHYEGPGGMLGGPEIMTDMFVKDIEQGIADTGVKAGILKCATDEPGITPGVERVLRAVAQAHKRTGVPISTHTHAGLRRGLEQQRIFEEEGVDLTRVVIGHSGDSTDIGYLEELIAAGSYLGMDRFGLDVISPFEERVKIVAQMCERGHADKMVLSHDANCYFDALPEELVPQMAPNWHYLHIHNDVIPALKQRGVTDEQLHTMLVDNPRRIFERQGAY

SEQ ID NO: 99 - protein, 325 aa, Mycobacterium simiae
MSELNTARGAIDTADLGVTLMHEHVFIMTTEIAQNYPEAWGDEEKRVADAIARLNELKARGVDTIVDLTVIGLGRYIPRIARVAAATELNIVVATGIYTYNDVPFRFHYEGPGGMLDGPEIMTEMFVRDIEQGIADTGVKAGILKCATDEPGITPGVERVLRAVAQAHKRTGVPISTHTHAGLRRGLDQQKIFDEEGVDLTRVVIGHSGDSTDVGYLEELIAAGSYLGMDRFGLDVISPFEERVKIVAQMCERGHADKMVLSHDANCYFDALPEELVPQLAPNWHYLHIHNDVIPALKERGVTDEQLHTMLVDNPRRIFERTGAY

SEQ ID NO: 100 - protein, 326 aa, Mycobacterium marinum
  misc_feature (84)..(84): Xaa can be any naturally occurring amino acid
MSALNTARGPIDTADLGVTLMHEHVFIMTTEVAQNYPEAWGDEEKRVADAITRLNELKSRGVDTIVDLTVIGLGRYIPRIARIXAATDLNIVVATGLYTYNDVPYRFHYQGPGGMLDGPEIMTDMFVRDIEQGIADTGVKAGILKCATDEPGITPGVERVLRAVAQTHKRTGVPISTHTHAGLRRGLEQQRIFEEEGVDLSRVIIGHSGDSTDVGYLEELIAAGSYLGMDRFGIDLILPFEDRVNIVATMCERGHADKMVLSHDANCYFDALPEELTAVATPNWHYLHIHNDVIPALKERGVTDEQVHTMLVDNPRRIFERQGGYQ

SEQ ID NO: 101 - protein, 328 aa, Mycobacterium sp.E796
MPMPELNTARGAIDTADLGVTLMHEHVFIMTTEIALNYPEAWGDEDQRVADAVNRLNELKSRGVDTIVDLTVIGLGRYIPRIARVAAATELNIVVATGLYTYNDVPFRFHYLGPGTQLDGPEIMTDMFVRDIEEGIADTGIKAGILKCATDEPGVTPGVERVLRAVAQAHKRTGVPISTHTHAGTRRGLEQQRIFEEEGVDLSRVVIGHSGDSTDLDYLEELIGNGSYIGMDRFGIDVYLPFEDRVNTVARMCERGHADKMVLSHDANCYFDALPEELLPVAAPNWHYLHIHNDVIPALKERGVTDEQLHTMLVDNPRRIFERQGAYS

SEQ ID NO: 102 - protein, 330 aa, Pseudomonas sp. WBC-3
MPLKNRLLARLSCVAAVVAATAAVAPLTLVSTAHAAAPQVRTSAPGYYRMLLGDFEITALSDGTVALPVDKRLNQPAPKTQSALAKSFQKAPLETSVTGYLVNTGSKLVLVDTGAAGLFGPTLGRLAANLKAAGYQPEQVDEIYITHMHPDHVGGLMVGEQLAFPNAVVRADQKEADFWLSQTNLDKAPDDESKGFFKGAMASLNPYVKAGKFKPFSGNTDLVPGIKALASHGHTPGHTTYVVESQGQKLALLGDLILVAAVQFDPSVTTQLDSDSKSVAVERKKAFADAAKGGYLIAASHLSFPGIGHIRAEGKGYRFVPVNYSVVNPK

SEQ ID NO: 103 - protein, 332 aa, Pseudomonas sp. diminuta
GDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRAS